(19) United States
(12) Patent Application Publication
(10) Pub. No.: US 2023/0037026 A1
(43) Pub. Date: Feb. 2, 2023

(54) METHOD FOR IDENTIFYING REGULATORY ELEMENTS CONFORMATIONALLY

(71) Applicant: ASKLEPIOS BIOPHARMACEUTICAL, INC., Research Triangle Park, NC (US)

(72) Inventors: Michael L. Roberts, Midlothian (GB); Richard Jude Samulski, Hillsborough, NC (US); Thomas Waibel, Midlothian (GB); Ross Fraser, Midlothian (GB); Joanna Critchley, Midlothian (GB); Kerstin Brzezek, Midlothian (GB)

(73) Assignee: ASKLEPIOS BIOPHARMACEUTICAL, INC., Research Triangle Park, NC (US)

(21) Appl. No.: 17/787,900

(22) PCT Filed: Dec. 23, 2020
(86) PCT No.: PCT/US2020/066766

§ 371 Date: Jun. 21, 2022

Related U.S. Application Data
(60) Provisional application No. 62/953,306, filed on Dec. 24, 2019.

(51) Int. Cl. C12N 15/113; C12N 15/86; C12N 15/10

(52) U.S. Cl. CPC C12N 15/113; C12N 15/86; C12N 15/1065; C12N 2750/14143; C12N 2750/14171; C12N 2740/15043; C12N 2740/15071

(57) ABSTRACT

The present invention provides a method of identifying the strength of one or more unique regulatory elements (URE) having conformational effect on a transcribable reporter sequence.

### CROSS-REFERENCE TO RELATED APPLICATIONS

This application is a U.S.C. § 371 National Phase Entry application of International Patent Application No. PCT/US2020/066766 filed on Dec. 23, 2020, which designated the U.S., which claims benefit under 35 U.S.C. § 119(e) of U.S. Provisional Application No. 62/953,306 filed Dec. 24, 2019, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Feb. 9, 2021, is named 046192-096050W0PT_SL.txt and is 9,512 bytes in size.

### FIELD OF THE INVENTION

The present invention relates to methods for identifying the strength of unique regulatory elements. In one embodiment, how conformational changes in the nucleic acid sequence effects the strength of such elements.

### BACKGROUND OF THE INVENTION

Regulatable gene expression is desirable in many circumstances, where it is beneficial or necessary to control the expression levels of an expression product. For example, in gene therapy it is desirable to induce expression of a therapeutic product (e.g., a therapeutic protein) at the desired level during a definite time and/or at a preferred location of treatment. In another example, in the case of industrial biotechnology, it can be highly advantageous to induce production of an expression product (e.g., a protein) at the desired time in a fermentation process.

Gene expression programs that drive development, differentiation, and many physiological processes are in large part encoded by DNA and RNA sequence elements that recruit regulatory proteins and their co-factors to specific genomic loci or genes under specific conditions. Despite significant research efforts, the relationship between the nucleic acid sequence and the function of these regulatory elements, such as cis-regulatory elements and trans-regulatory elements, remains poorly understood. For example, the placement of such elements; repeating elements, adding elements, the spacing between elements, the spacing with open reading frames, the spacing with respect to 5′ and 3′ ends, etc. This limited understanding of these regulatory elements is an impediment to a variety of fields, including synthetic biology, medical genetics, and evolutionary biology. There are also differences in expression between different cell types. Differences can exist between in vitro and in vivo systems.

Thus, more efficient approaches to elucidate the relationship between DNA sequences encoding, e.g., regulatory elements, cells, expression systems, and the function of regulatory elements, are needed.

### SUMMARY OF INVENTION

The overall 3-dimensional structure (conformation) of nucleic acid sequences such as viral vectors can change depending upon different microenvironments where the sequence is, and/or mutations, deletions, additions, and substitutions of the sequence. One aspect of the invention described herein provides a method of identifying the strength of one or more unique regulatory elements (URE) and the effect of the overall conformation of the nucleic acid sequence the URE is present within relative to a transcribable reporter sequence, such as an open reading frame (ORF) comprising (a) expressing a plurality of synthetic nucleic acid sequences in a population of cells, the plurality of synthetic nucleic acid sequences comprises (1) a first plurality of synthetic nucleic acid sequences each comprising a unique regulatory element (URE) wherein the URE comprises (i) a nucleic acid sequence containing at least one discrete regulatory element (DRE), wherein the DRE is a control (or wild type) continuous nucleic acid sequence or a control discontinuous nucleic acid sequence associated with a plurality of unique barcodes corresponding with the at least one DRE, wherein each barcode is between 12-35 nucleotides in length and has a GC content between 25-65%; and (ii) the DRE is conformationally positioned in a preselected manner relative to a nucleic acid encoding a transcribable reporter sequence, wherein if the URE does not contain a promoter, a separate promoter is operatively linked to the transcribable reporter sequence; and (2) a second plurality of synthetic nucleic acid sequences comprising a URE that further comprises a change in the conformation of the sequences relative to at least one DRE of a(1)(ii) with respect to the transcribable reporter sequence wherein the DRE in the conformationally changed sequence is associated with a plurality of unique barcodes different than in (1)(i), wherein each barcode is between 12-35 nucleotides in length and has a GC content between 25-65%; (b) determining the expression frequency of each of the plurality of corresponding barcodes in (a)(1) and (a)(2); to determine the effect of the conformational change. In a further embodiment, the above method further comprises (c) changing in a predetermined manner the conformation of at least one of the corresponding plurality of synthetic nucleic acids relative to the DRE and the transcribable reporter sequence; (d) determining the expression frequency of the at least one corresponding plurality of (c); and (e) comparing the expression frequency of (a)(1) and (a)(2) to determine the effect of the conformation change on the transcribable reporter sequence expression.

In an alternative embodiment, the transcribable reporter sequence is not present.

In an alternative embodiment, the transcribable reporter sequence is an ORF. In one embodiment, the ORF is a gene.

In one embodiment of any aspect described herein, the plurality of synthetic nucleic acids is expressed in a population of cells using a population of viral vectors.

In one embodiment of any aspect described herein, the DRE is proximal to or within a Holliday junction and a change in at least one of the Holliday junctions is made.

In one embodiment of any aspect described herein, the change in conformation is made by the addition, deletion, or substitution of one or more nucleic acids.

In one embodiment of any aspect described herein, at least one DRE is present in a terminal repeat (TR).

In one embodiment of any aspect described herein, the viral vector is a parvovirus, a lentivirus, or an adenovirus.

In one embodiment of any aspect described herein, the parvovirus is a dependovirus and the change in conformation is in at least one of the A, A′, B, B′, C, or C′ loops.

In one embodiment of any aspect described herein, the parvovirus is an adeno-associated virus (AAV) and the change in conformational is in at least one of the A, A′, B, B′, C, C′, D, D′ regions.

In one embodiment of any aspect described herein, the viral vector is a lentiviral vector, the DRE is TAT, and the conformational change is made in the TAR RNA stem.

In one embodiment of any aspect described herein, the viral vector is a lentiviral vector, the DRE is TAT, and the conformational change is made in the U-rich bulge in the TAR RNA stem.

In one embodiment of any aspect described herein, the viral vector is a lentiviral vector, the DRE is REV, a REV Responsive Element (RRE) is present in the nucleic acid, and the conformational change is made in the RRE.

In one embodiment of any aspect described herein, the DRE is proximal to or within the conformation change.

In one embodiment of any aspect described herein, the conformational change occurs by the addition, substitution, or deletion of at least one nucleic acid.

In one embodiment of any aspect described herein, the addition, substitution, or deletion results in a Holliday junction.

In one embodiment of any aspect described herein, the plurality of synthetic nucleic acids is expressed in a population of cells in vitro using a population of AAV vectors.

In one embodiment of any aspect described herein, the plurality of synthetic nucleic acids is expressed in a population of cells in vivo using a population of AAV vectors.

A method of identifying the strength of one or more unique regulatory elements (URE) having conformational effect on a transcribable reporter sequence comprising (a) providing a plurality of synthetic nucleic acids, wherein the plurality of synthetic nucleic acid comprises (1) a first plurality of synthetic nucleic acids each comprising a unique regulatory element (URE), wherein the URE comprises (i) a nucleic acid sequence containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is conformationally positioned in a preselected manner relative to a nucleic acid encoding a transcribable reporter sequence operatively linked to a promoter; wherein if the URE does not contain a promoter, a separate promoter is operatively linked to the transcribable reporter sequence; and (2) a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%; (b) generating a library of plasmids or expression vectors by inserting the plurality of synthetic nucleic acids into a plurality of plasmids or expression vectors, wherein each resulting plasmid or expression vector comprises a single synthetic nucleic acid; (c) introducing the library of plasmids or expression vectors of step (b) into a population of cells; (d) determining the expression frequency of each of the plurality of corresponding barcodes in (a) (1) and (a) (2); and (e) comparing the expression frequency of (a)(1) and (a)(2) to determine the effect of the conformation change on the transcribable reporter sequence expression. A skilled artisan can learn the necessity for certain sequences or conformations as a result of a reduction in the amount of amplification of the amplicon. Enhanced amplification indicates improvements by these changes. Alternatively, loss of amplification indicates the necessity of the changed sequence or conformation.

A method of identifying the conformational effect on one or more unique regulatory elements (URE) associated with a transcribable reporter sequence comprising (a) providing the plurality of nucleic acids, wherein the plurality of synthetic nucleic acid comprises (1) a unique regulatory element (URE), wherein the URE comprises (i) a first plurality of synthetic nucleic acid sequences each containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is positioned in a preselected manner relative to a nucleic acid encoding a transcribable reporter sequence operatively linked to a promoter; wherein if the URE does not contain a promoter, a separate promoter is operatively linked to the transcribable reporter sequence; and (2) a second plurality of synthetic nucleic acids comprising a URE further comprising a change in the conformation of said nucleic acids a(1)(ii) relative to the at least one DRE associated with the transcribable reporter sequence wherein the DRE in the conformationally changed sequence is associated with a plurality of unique barcodes different than in (1)(i), wherein each barcode is between 12-35 nucleotides in length and has a GC content between 25-65%; (b) generating a library of plasmids or expression vectors by inserting the plurality of synthetic nucleic acids into a plurality of plasmids or expression vectors, wherein each resulting plasmid or expression vector comprises a single synthetic nucleic acid; (c) introducing the library of plasmids or expression vectors of step (b) into an AAV vector to form an AAV vector library; (d) introducing the AAV vector library into a population of cells; (e) determining the expression frequency of each of the corresponding barcodes of (a)(1) and (a)(2); and (f) comparing the expression frequency of (a)(1) and (a)(2) to determine the effect of the conformation change on the strength of expression.

In one embodiment of any aspect described herein, the method further comprises the step of, after step (a), waiting a sufficient amount of time for expression of the transcribable reporter sequence, e.g., an open reading frame such as a marker protein or fluorescent protein, in the population of cells.

In one embodiment of any aspect described herein, the method further comprises the step of, after step (c), waiting a sufficient amount of time for expression of the library of plasmids or expression vectors of step (b).

In one embodiment of any aspect described herein, determining the expression frequency of the barcode unique to a specific URE includes the steps of: (a) obtaining a transcript, e.g., an mRNA transcript, from the population of cells or the population of AAV vectors; (b) synthesizing cDNA from the mRNA of step (a); (c) amplifying a region of nucleic acids (amplicon) from the cDNA of step (b); and (d) measuring the expression frequency of the plurality of barcodes in the amplicon of step (c).

In one embodiment of any aspect described herein, determining the expression frequency includes the steps of: obtaining mRNA from tissues or cells of interest after in vivo administration of viral vectors; synthesizing cDNA from the mRNA of step (a); amplifying a region of nucleic acids (amplicon) from the cDNA of step (b); and measuring the expression frequency of each of the plurality of barcodes in the amplicon of step (c). In an alternate embodiment, determining the expression frequency includes the steps of: obtaining a transcript from tissues or cells of interest after in vivo administration of viral vectors; synthesizing cDNA from the transcript of step (a); amplifying a region of nucleic acids (amplicon) from the cDNA of step (b); and measuring the expression frequency of each of the plurality of barcodes in the amplicon, or population thereof of step (c). A transcript useful for determine are transcripts that can serve as a template for cDNA synthesis, for example, microRNA. One skilled in the art can identify and obtain a transcript for cDNA synthesis, as described herein.

In one embodiment of any aspect described herein, measuring is performed by sequencing.

In one embodiment of any aspect described herein, the expression frequency of each of the plurality of barcodes is the normalized to a barcode input, and wherein the barcode input is each unique barcode content before expression. In one embodiment of any aspect described herein, the expression frequency of the barcode measured in the amplicon, or population thereof, is a barcode output.

In one embodiment of any aspect described herein, at least one DRE is a discontinuous DRE.

In one embodiment of any aspect described herein, the discontinuous DRE comprises a portion of the DRE located 5′ of the transcribable reporter sequence, and a portion of the DRE located 3′ of the transcribable reporter sequence. In one embodiment of any aspect described herein, the discontinuous DRE comprises a non-DRE nucleic acid sequence located in a 5′- or 3′-portion of the DRE.

In one embodiment of any aspect described herein, the at least one DRE is located within 200-500 bp of the at least one TR, or portion thereof. In one embodiment of any aspect described herein, the at least one DRE is located within 20-200 bp of the at least one TR, or portion thereof. In one embodiment of any aspect described herein, the at least one DRE is located within 20 bp of the at least one TR, or portion thereof.

In one embodiment of any aspect described herein, the URE strength is measured in the same system from which it is derived.

In one embodiment of any aspect described herein, at least part of the at least one discontinuous DRE includes a TR. In one embodiment of any aspect described herein, the at least one TR, or portion thereof, comprises at least one modification. In one embodiment of any aspect described herein, the at least one TR comprises at least 1, 2, 3, 4, 5, 6, or more modifications.

In one embodiment of any aspect described herein, the at least 1, 2, 3, 4, 5, 6, or more modifications are associated with the same plurality of unique barcodes.

In one embodiment of any aspect described herein, the synthetic nucleic acid contains at least 2, 3, 4, 5, 6, or more TRs, or portion thereof. In one embodiment of any aspect described herein, the synthetic nucleic acid contains at least 2, 3, 4, 5, 6, or more discontinuous DREs.

In one embodiment of any aspect described herein, the URE comprises at least one DRE selected from a promoter, a transcription factor binding site, an enhancer, a silencer, a boundary control element, an insulator, a locus control region, a response element, a binding site, a segment of a terminal repeat, a responsive site, a stabilizing element, a de-stabilizing element, or a splicing element.

In one embodiment of any aspect described herein, the nucleic acid sequence containing at least one DRE comprises a combination of DREs. In one embodiment of any aspect described herein, the combination of DREs contain at least 2, 3, 4, 5, 6, or more regulatory sequence elements.

In one embodiment of any aspect described herein, the combination of DREs is associated with the same plurality of unique barcodes described herein.

In one embodiment of any aspect described herein, the viral vector is selected from an AAV vector, an adenovirus vector, a lentivirus vector, a retrovirus vector, a herpesvirus vector, an alphavirus vector, a poxvirus vector, a baculovirus vector, and a chimeric virus vector. In one embodiment of any aspect described herein, the AAV vector is a AAV serotype selected from the group consisting of: 1, 2, 3a, 3b, 4, 5, 6, 7, 8, 9, 10, 11, and 13.

In one embodiment of any aspect described herein, the synthetic nucleic acid comprises an inverted terminal repeat (ITR), or a portion thereof.

In one embodiment of any aspect described herein, the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a TRS (terminal resolution site), and a Rep binding site (RBS).

In one embodiment of any aspect described herein, the ITR is a wild-type inverted terminal repeat (ITR), a mutant ITR, or a synthetic ITR, wherein the mutant or synthetic ITR comprises a modification as compared to the wild-type ITR sequence.

In one embodiment of any aspect described herein, the A region, A′ region, B region, B′ region, C region, C′ region, D region, or D′ region is derived from a wild-type inverted terminal repeat (ITR), a mutant ITR, a truncated ITR, or a synthetic ITR.

In one embodiment of any aspect described herein, the TR is a long terminal repeat (LTR), or a portion thereof.

In one embodiment of any aspect described herein, the modification is a base pair insertion, deletion, mutation, truncation, or substitution as compared to the wild-type ITR sequence.

In one embodiment of any aspect described herein, the at least one DRE and the TR sequence are separated by 1-500 base pairs.

In one embodiment of any aspect described herein, each portion of a discontinuous DRE (dcDRE) is separated by 1-500 base pairs. In one embodiment of any aspect described herein, each portion of a discontinuous DRE (dcDRE) is separated by at least 50 base pairs.

In one embodiment of any aspect described herein, one portion of a discontinuous DRE (dcDRE) can be 5′ of the transcribable reporter sequence, and a second portion of the dcDRE is 3′ of the transcribable reporter sequence.

In one embodiment of any aspect described herein, the transcribable reporter sequence is an open reading frame (ORF). In one embodiment, the ORF of a marker gene. Exemplary marker genes include genes encoding a fluorescent protein, a luminescent protein, or an element tag. In one embodiment, the ORF is a therapeutic gene.

In one embodiment of any aspect described herein, the barcode contains at least one of each: adenine, thymine, guanine, and cytosine.

In one embodiment of any aspect described herein, the barcode is a semi-degenerate barcode.

In one embodiment of any aspect described herein, the barcode does not contain tracts of more than three homopolymers in succession.

In one embodiment of any aspect described herein, the barcode does not contain the nucleic acid sequence of a restriction enzyme.

In one embodiment of any aspect described herein, the barcode has a hamming distance greater than 2 when compared to other barcodes within the plurality of barcodes.

In various embodiments of any aspect described herein, the barcode is between 12-25 nucleotides in length, or between 12-28 nucleotides in length. In one embodiment of any aspect described herein, a plurality of barcodes comprises 2-20 barcodes. For example, the plurality of barcodes comprises at least 2, at least 3, at least 4, at least 5, at least 6, at least 7, at least 8, at least 9, at least 10, or more barcodes, or 2-6 barcodes.

In one embodiment of any aspect described herein, the synthetic nucleic acid is further modified for next generation sequencing. In one embodiment of any aspect described herein, the synthetic nucleic acid comprises at least one unique molecular identifier (UMI) and at least one unique primer annealing sites (UPAS) tag.

In one embodiment of any aspect described herein, the conformational change is not determined. Alternatively, in one embodiment, the conformational change determined by assessing the at least one mutation against a non-altered sequence under the same condition.

Another aspect described herein provides a plurality of at least 50 synthetic nucleic acids, each synthetic nucleic acid comprising a URE, where the URE comprises (a) a nucleic acid sequence containing at least one discrete regulatory element (DRE), wherein the DRE is a continuous nucleic acid sequence or a discontinuous nucleic acid sequence; (b) a nucleic acid sequence encoding an open reading frame; (c) a nucleic acid sequence encoding a viral vector terminal repeat (TR); and (d) a plurality of unique barcodes associated with the at least one DRE, wherein each barcode has a GC content between 25-65%.

In one embodiment of any aspect described herein, the barcode when part of a plurality of nucleic acid sequence, has a complexity of at least 4.3×10⁷, at least 2.7×10⁸, or at least 1×10¹². In another embodiment of any aspect described herein, the plurality of barcodes has a complexity of at least 4.3×10⁷, at least 2.7×10⁸, or at least 1×10¹².

Another aspect described herein provides a plurality of at least 50 synthetic nucleic acids, each synthetic nucleic acid comprising a URE, where the URE comprises (a) a nucleic acid sequence containing at least one discrete regulatory element (DRE), wherein the DRE is a continuous nucleic acid sequence or a discontinuous nucleic acid sequence; (b) a nucleic acid sequence encoding an open reading frame; (c) a nucleic acid sequence encoding at least one partial viral vector comprising at least a part of a terminal repeat (TR); and (d) a plurality of unique barcodes associated with the at least one DRE, wherein each barcode is between 12-35 nucleotides in length and have a GC content between 25-65%.

In one embodiment of any aspect described herein, the DRE comprises at least one regulatory sequence element selected from a promoter, a transcription factor binding site, an enhancer, a silencer, a boundary control element, an insulator, a locus control region, a response element, a binding site, a segment of a terminal repeat, a responsive site, a stabilizing element, a de-stabilizing element, and a splicing element.

In one embodiment of any aspect described herein, at least part of the at least one DRE includes a TR.

In one embodiment of any aspect described herein, in the synthetic nucleic acid contains at least 2 TRs.

In one embodiment of any aspect described herein, the at least one discontinuous regulatory element comprises at least one modification.

In one embodiment of any aspect described herein, the viral vector comprises at least 4 modifications.

In one embodiment of any aspect described herein, the TR is an inverted terminal repeat (ITR).

In one embodiment of any aspect described herein, the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a spacer sequence, a CAP gene sequence, a Rep gene sequence, a Rep Binding Site, and a terminal resolution site.

Another aspect described herein provides a library of at least 50 plasmids expressing any of the plurality of synthetic nucleic acids described herein.

Another aspect described herein provides a library of at least 50 expression vectors comprising any of the plurality of synthetic nucleic acids described herein.

In one embodiment of any aspect described herein, the library comprises control plasmids or control expression vectors.

Another aspect described herein provides a population of cells comprising any of the libraries described herein.

In one embodiment of any aspect described herein, the cells are eukaryotic, prokaryotic, viral, or bacterial.

In various embodiments of any aspect described herein, the synthetic nucleic acids, plasmids, or expression vectors is transiently expressed or stably expressed.

Another aspect described herein provides a population of at least 50 viral vectors expressing any of the plurality of synthetic nucleic acids described herein, any of the libraries of plasmids described herein, or any of the libraries of expression vectors described herein. In one embodiment of any aspect described herein, the viral vector is an AAV vector.

Another aspect described herein provides a method of identifying the strength of a URE from a plurality of UREs in vitro, the method comprising (a) expressing any of the plurality of synthetic nucleic acids described herein, any of the libraries of plasmids described herein, or any of the libraries of expression vectors described herein in a population of cells; and (b) determining the expression frequency of each of the plurality of barcodes, wherein the expression frequency of each of the plurality of barcodes is an indicator of the strength of the associated URE.

Another aspect described herein provides a method of identifying the strength of a URE from a plurality of UREs in vitro, the method comprising (a) providing any of the plurality of synthetic nucleic acids described herein; (b) inserting the plurality of synthetic nucleic acids into a library of plasmids or expression vectors, wherein the resulting plasmid or expression vector each comprise at least one DRE, an open reading frame, a viral vector terminal repeat (TR) or at least one partial viral vector comprising at least a part of a terminal repeat (TR), and a plurality of barcodes associated with at least one DRE; (c) introducing the library of plasmids or expression vectors of step (b) into a population of cells; and (d) determining the expression frequency of the plurality of barcodes, wherein the expression frequency of each of the plurality of barcodes is an indicator of strength of the URE.

Another aspect described herein provides a method of identifying the strength of a URE from a plurality of UREs in vitro, the method comprising (a) providing any of the pluralities of synthetic nucleic acids described herein; inserting the plurality of synthetic nucleic acids into a library of plasmids or expression vectors, wherein the resulting plasmid or expression vector each comprises at least one DRE, an open reading frame, a viral vector terminal repeat (TR) or at least one partial viral vector comprising at least a part of a terminal repeat (TR), and a plurality of barcodes associated with the at least one DRE; (b) introducing the plurality of plasmids or expression vectors of step (a) into an AAV vector to form AAV vector library; (c) introducing the AAV vector library into a population of cells; and (d) determining the expression frequency of the plurality of barcodes, wherein the expression frequency of each of the plurality of barcodes is an indicator of the strength of the URE.

In one embodiment of any aspect described herein, the method further comprises the step of, prior to determining, waiting a sufficient amount of time for expression of the synthetic nucleic acids, the plasmids, or the expression vectors.

Another aspect described herein provides a method of identifying the strength of a URE from a plurality of UREs in vivo, the method comprising (a) administering any of the populations of viral vectors described herein in vivo; and (b) determining the expression frequency of each of the plurality of barcodes, wherein the expression frequency of each of the plurality of barcodes is an indicator of the strength of the associated URE.

Another aspect described herein provides a method of identifying the strength of a URE from a plurality of UREs, the method comprising (a) providing any of the pluralities of synthetic nucleic acids described herein; (b) inserting the plurality of synthetic nucleic acids into a library of plasmids or expression vectors, wherein the resulting plasmid or expression vector each comprise a single synthetic nucleic acid; (c) introducing the plurality of plasmids or expression vectors of step (b) into an viral vector; (d) administering the resulting viral vector of step (c) in vivo; and (d) determining the expression frequency of each of the plurality of barcodes, wherein the expression frequency of each of the plurality of barcodes is an indicator of the strength of the associated URE.

In one embodiment of any aspect described herein, the method further comprises the step of, after administering, waiting a sufficient amount of time for expression of the synthetic nucleic acids, the plasmids, or the expression vectors.

Another aspect provides a plurality of at least 50 synthetic nucleic acids, each synthetic nucleic acid comprising a URE, where the URE comprises (a) a nucleic acid sequence containing at least one discrete regulatory element (DRE), wherein the DRE is a continuous nucleic acid sequence or a discontinuous nucleic acid sequence; (b) a nucleic acid sequence encoding an open reading frame; (c) a nucleic acid sequence encoding a viral vector terminal repeat (TR); and (d) a plurality of unique barcodes associated with the at least one DRE, wherein each barcode has a GC content between 25-65%.

Another aspect provides a plurality of at least 50 synthetic nucleic acids, each synthetic nucleic acid comprising a URE, where the URE comprises (a) a nucleic acid sequence containing at least one discrete regulatory element (DRE), wherein the DRE is a continuous nucleic acid sequence or a discontinuous nucleic acid sequence; (b) a nucleic acid sequence encoding an open reading frame; (c) a nucleic acid sequence encoding at least one partial viral vector comprising at least a part of a terminal repeat (TR); and (d) a plurality of unique barcodes associated with the at least one DRE, wherein each barcode is between 12-35 nucleotides in length and has a GC content between 25-65%.

In one embodiment of any aspect described herein, the viral vector comprises 1-6 modifications, e.g., 1, 2, 3, 4, 5, or 6 modifications. In one embodiment of any aspect described herein, the 1-6 modifications are associated with the same plurality of unique barcodes as described herein above.

In one embodiment of any aspect described herein, the partial viral vector is selected from a terminal repeat, response element, cis-acting viral element, and a trans-acting viral element.

In all embodiments, a conformational change can be determined by any means known in the art. For example, comparing the change in activity to a “control” conformation. In another embodiment, exemplar conformations are used as a standard, with the change compared under like conditions to that of the exemplar.

### BRIEF DESCRIPTION OF THE DRAWINGS

FIG. 1 is a schematic representation of exemplary cloning steps to generate a library of synthetic nucleic acids, each synthetic nucleic acid comprising a regulatory element (referred to as synthetic promoter library in the figure), a minimal promoter (MP) linked with an ORF comprising a reporter gene, and a plurality of unique barcodes at the 3′ end of the ORF. In Step 1, the regulatory element was cloned (obtained as described herein below in FIG. 8) into the screening vector backbone, Step 2 added the plurality of barcodes to the vector backbone, and step 3 added the minimal promoter linked with an ORF to the same vector so that it was placed in between the regulatory element and the plurality of barcodes. Exemplary ORFs included reporter genes such as SEAP and GFP.

FIG. 2 is a schematic representation of the High Content Screening Assay (HCS) using the expression frequency of the barcode to determine the strength of the URE. Briefly, the strength of URE is determined from the barcode sequencing, wherein one or more barcodes, e.g., a plurality, are unique to the specific regulatory element. The URE transfection and the amplicon generation was performed as described in FIG. 3 and as shown in the box on the right panel of this figure. The barcode sequence obtained from the amplicon was normalized to the barcode content in the plasmid DNA or the genomic DNA (gDNA) before expression i.e., before transfection to cells. The normalized ratio or the barcode ratio corresponded to the strength of the URE and thus led to the promoter/URE discovery by HCS assay.

FIG. 3 is a schematic representation of amplicon generation followed by sequencing of the plurality of barcodes after transfection of the library of synthetic nucleic acids comprising regulatory elements as disclosed herein in an in vitro system. Briefly, the library was transfected into the cells followed by the harvesting of cells, extraction of RNA, synthesis of cDNA and finally amplification of the cDNA. Primers for amplicon generation included multiplexing index primer with the sequencing primers, i.e., P7 and P5 oligo primers. FIG. 3 discloses SEQ ID NOS 29-30, respectively, in order of appearance.

FIG. 4 is a schematic representation of production of viral vectors (AAV vectors) comprising the library of synthetic nucleic acids comprising UREs as disclosed herein. AAV libraries are constructed using an interim cloning vector. Exemplary UREs in the AAV library pool were multiple tissue-specific enhancer tiles. Followed by the AAV injection in mice, enhancer modules were identified by identifying active CREs. Data-driven design of numerous promoters were then performed and these were finally validated in mice.

FIG. 5 is a schematic representation of the generation AAV viral vectors for in vivo validation of the UREs (referred to as “candidate CRE”). Nucleic acid sequences comprising UREs comprising a unique barcode were cloned into an interim vector and then a minimal promoter (MP) linked with ORF (encoding GFP) was further cloned into the interim vector between the URE and BC to generate the synthetic nucleic acids as disclosed herein. The synthetic nucleic acid construct was cloned into an AAV vector to form a AAV vector library. AAV library was introduced into cell followed by lysis of cells, purification of AAV particles and thus generating the AAV preparation (designated as AAV prep) in the figure. Purified AAV vector comprising the synthetic nucleic acid or AAV prep as disclosed herein was used in an in vivo screen.

FIG. 6 is a schematic diagram of an exemplary in vivo high content screening assay to assess the tissue specificity and/or strength of the URE. TFBSs are identified from differentially expressed genes in the genome. Complex shuffled libraries are then constructed comprising these TFBSs. The barcode content in the AAV preparation prior to injection (input BC sequencing) and the frequency of the expression of the barcode in specific tissues after AAV injection in vivo (output BC sequencing) were determined to assess the strength and specificity of the URE in specific tissues in vivo.

FIG. 7 is a schematic representation of the generation of exemplary UREs. Using RNASeq data and bioinformatics, the promoter regions of highly expressed stable genes were identified, and assessed to identify CRE regions (CRE refers to cis-regulatory element). DNA fragments with identified CREs were digested with restriction enzymes to generate numerous fragments harboring individual, combination or a pool of transcription factor binding sites (TFBS). These fragments of DNA harboring TFBSs were then excised from gel and ligated to specific adapters to generate UREs (referred herein as synthetic promoter (SP) constructs). FIG. 7 discloses SEQ ID NO: 31.

FIG. 8 is another schematic of the generation of exemplary UREs, showing identification of restriction sites in the CRE (e.g., E1, E2, E3, etc.) and sequential digestion by the restriction enzymes and subsequent random assembly of the fragments to generate an exemplary URE. The exemplary URE is them cloned into the vector as described herein above in FIG. 1.

FIGS. 9A-9E shows analysis of a library of synthetic nucleic acids as disclosed herein in HK4 cells. FIG. 9A shows equal representation of all TFBS in the library. FIG. 9B shows that in a library of more than 178,000 synthetic nucleic acids, each nucleic acid construct comprises on average 3.9 barcodes linked to each URE (SP). FIG. 9C shows that each URE in the library comprises on average 4-6 TFBMs. FIG. 9D shows that 91.8% of the barcodes are associated with only one URE. FIG. 9E shows that there are 705,746 distinct URE-BC pairs, with an average of 6.4 barcodes per URE.

FIG. 10 shows exemplary barcoding strategies, including random barcodes, semi-degenerate barcodes and barcodes for in vivo screening of the UREs. In some embodiments, the plurality of barcodes had a complexity of >1×10¹², or where 20 different pools of barcodes are available, the barcode ha a complexity of >4.3×10⁷. In some embodiments, the plurality of barcode had any one or more of: comprising a homopolymer of <3, GC content of >0.25 and <0.65, containing all 4 nucleotides, and did not comprise a restriction endonuclease recognition site, had a hamming distance of >2 and complexity of >2.8×10⁸. FIG. 10 discloses SEQ ID NOS 32-34, respectively, in order of appearance.

FIG. 11 shows assessment of exemplary UREs comprising a repeated regulatory element primary hepatocytes in vitro. The UREs comprise a different number of the same repeated regulatory element (represented as “enhancer 1”) which was located 5′ of each of the four minimal promoters (MP1-4) and together were placed upstream of an ORF encoding the luciferase gene. The expression level of luciferase in primary hepatocytes before and after addition of an inducing agent are shown in grey and blue respectively.

FIGS. 12A-12B shows the assessment of exemplary UREs comprising a repeated regulatory element primary hepatocytes in vitro to determine robustness of the URE. The UREs comprise a different number of the same repeated regulatory element (represented as “enhancer 1”) which was located 5′ of each of the four minimal promoters (MP1-4) and together were placed upstream of an ORF encoding the EPO gene, which is an exemplary expression product or therapeutic gene. The expression level of EPO in primary hepatocytes on different concentrations of an inducer (FIG. 12A) or before and after addition of an inducing agent are shown in grey and blue respectively (FIG. 12B).

FIG. 13 shows the assessment of exemplary UREs comprising a repeated regulatory element in different cells in vitro to determine tissue specificity and robustness of the URE. The UREs comprise a different number of the same repeated regulatory element (represented as “enhancer 1”) which is located 5′ of each of the four minimal promoters (MP1-4) and together were placed upstream of an ORF encoding luciferase. The expression level of luciferase was normalized to the expression from the CMV-IE promoter in primary hepatocytes and HEK cells before and after addition of an inducing agent are shown in grey and blue respectively. The result shows that one particular URE driven expression was remarkably less both in primary cells and in HEK 293 cells, whereas the other URE driven expression was significantly high in primary hepatocytes when compared with that in HEK 293 cells.

FIG. 14 shows the schematic of tagging barcodes with UPAS and UMI sequences such that the barcode can be amplified via illumine sequencing, e.g., with illumine adapters. Amplicons are generated via illumina sequencing primers and the frequency of the amplicons is measured. through sequencing. This approach is used to counter the stochasticity of PCR. FIG. 14 discloses SEQ ID NOS 29-30, respectively, in order of appearance.

FIG. 15 shows an overview of library cloning. The synthesized DNA string containing the individual TFBS (cis elements) are liberated by restriction enzyme digest and re-ligated to form synthetic promoters. A PCR adds specific overhangs allowing the integration into the screening vector using InFusion cloning. Size distribution of individual library constructs is shown.

FIG. 16 shows GFP positive CHO-S cells and mean GFP intensity post library transfection. Two different carrier plasmids, pShuttle and pMK-RQ are used. Both the number of GFP positive cells and the mean GFP intensity is increased post HK4 library transfection when compared to the CMV minimal promoter indicating the functionality of the HK4 library in CHO-S cells.

FIG. 17 shows barcode distribution and promoter activity of controls and shuffled library determined by HCS. The nine boxplots represent five biological replicates 24 h post transfection and four replicates 48 h post transfection. Each control data point, namely CMV-IE, CMVmp, EF1alpha, promoterless EGFP and PGK, is the mean frequency of seven individual barcodes. Frequencies of shuffled library barcodes are shown on the right.

FIG. 18 shows synthetic promoter selection criteria workflow. Specific parameters are applied as filters to select the core candidate promoters

FIG. 19 shows scatter plot of 20,586 selected synthetic promoters. Candidate promoters with low variance are selected for validation of the HCS method (right hand magnification).

FIGS. 20A and 20B show barcode variation of synthetic and control promoters. (FIG. 20A) Variation of the same barcode of a synthetic promoter. (FIG. 20B) Variation of the same barcode of CMV-IE. Barcode variation of synthetic promoters is noted to be greater when compared with control promoters. Barcode variations are shown across all 9 replicates representing 24 h (1-5) and 48 h (6-9) post transfection.

FIG. 21 shows expression levels of 8 selected candidate promoters. Luciferase expression levels relative to the CMV-IE promoter indicate the functionality of the HCS screen. All promoters are functional and show approximate expression levels within the expected range.

FIG. 22 shows a schematic of self-complementary AAV vector comprising two barcoded synthetic nucleic acids packaged into the vector; the first synthetic nucleic acid driven by the promoter of interest, and the second synthetic nucleic acid by a weak constitutive promoter. The barcodes of each synthetic nucleic acid promoter and normaliser are linked. Each synthetic nucleic acid contains one of two fluorescent proteins, e.g., green fluorescent protein or cherry fluorescent protein.

FIG. 23 shows a schematic of in vivo high content screening. A plurality of barcoded synthetic nucleic acids is administered to a mammalian subject, e.g., a mouse, and expression of each of the barcoded synthetic nucleic acids are assessed via next generation sequence in a selected organ or tissue type. in vivo high content screening can be used to determine promoter activity that is specific for a given organ or tissue type. The mode of administration is selected based on the target tissue or organ, e.g., intra-cerebral injection is used to achieve expression of the plurality of barcoded synthetic nucleic acids in the brain.

FIG. 24 shows a graph depicting the approximately 9 million reads produced from PacBio library preparation and sequences on the PacBio Sequel platform by Edinburgh Genomics. A median length of ˜2200 base pairs.

FIG. 25 shows schematic of PacBio read structure terminology. PacBio reads are made up of Polymerase reads and Subreads.

FIG. 26 shows number of library barcodes per polymerase ID. Plot generated from 100,000 Subreads. Graph shows the number of unique barcodes found per polymerase, and total number of barcodes per polymerase read.

FIG. 27 shows a schematic of the cloning process of generating multiple barcodes using compatible restriction sites. The original construct combines all three barcodes which are selectively excised by restriction endonuclease digestion and relegation.

### DETAILED DESCRIPTION OF THE INVENTION

In general, the invention described herein provides synthetic nucleic acids, plasmids, expression vectors, cells, viral vectors, and simple yet efficient methods for identifying and classifying the how the conformation of a vector, e.g., a viral vector, effects the strength and/or tissue specificity of a unique regulatory element (URE), which has been distinctly tagged using a plurality of unique barcodes. The described unique barcodes provide a means to identify and categorize the discrete regulatory elements comprised in an individual cell or viral vector within a plurality of cells or viral vectors. Provided herein are synthetic nucleic acids, plasmids, expression vectors, cells, viral vectors, and methods for identifying how the conformation of a vectors effects the strength of a URE both in vitro and in an in vivo model; the conformation of the vector can differentially effect the URE performances in an in vitro versus in vivo system. While fluorescent proteins can be used in vitro, they are problematic in screening the function of UREs in vivo. A regulatory element may behave differently depending on the placement of the regulatory element relative to other sequences in the system, such as how far upstream or downstream a regulatory element is, where the above said sequences can be the gene, a terminal repeat, another regulatory element or a combination of regulatory elements. Our methodology permits rapid screening of UREs both in vitro and in vivo in vectors that are modified to induce a conformational change in the vector. This can be accomplished by screening for the amplification of a plurality of barcodes where the plurality of barcodes is operably linked to a specific regulatory element.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “plurality of synthetic nucleic acids” refers to an undivided sample that contains at least two or more (e.g., 50, 100, 1000, 5000, 10000, 15000, 25000, or more) distinct synthetic nucleic acids.

As used herein, the terms “nucleotide sequence”, “nucleic acid sequence”, and “DNA sequence,” are used interchangeably herein and refer to a sequence of a nucleic acid, e.g., a circular nucleic acid that is to be delivered into a target cell. Generally, the nucleic acid sequence comprises at least one URE, a transcribable reporter sequence, e.g., an open reading frame that encodes a polypeptide of interest (e.g., a marker gene), and at least one unique barcode. Preferably the nucleic acid is homologous, that is naturally occurring, in conjunction with the URE (e.g. naturally occurring in a cell from which the regulatory element is derived); such a nucleic acid is referred to as heterologous.

As used herein, “synthetic” refers to a continuous sequence of nucleotides that is not naturally occurring. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic URE (or portion of a regulatory element) typically contains one or more nucleic acid sequences that are not contiguous in nature (chimeric sequences), and/or may encompass substitutions, insertions, and deletions and combinations thereof.

As used herein, “unique regulatory element” or “URE” refers to at least one “regulatory elements”, which operate in part, or in whole, to regulate expression of a gene from a transcribable reporter sequence, e.g., an open reading frame (ORF). The URE, as disclosed herein, is a regulatory element coupled with a unique identifying barcode sequence or a plurality of barcode sequences. The URE can be a combination of regulatory elements. In some instances, an element when by itself or with other regulatory elements has no effect on transcription. Such elements are only effective in relation to other regulatory elements. When screening those such elements, they should be compared to an “active” combination of elements. The regulatory elements, when oriented and in an optimal configuration or operably linked, act together to modulate the activity of one another, and ultimately may affect the level of expression of an expression product encoded by the transcribable reporter sequence, e.g., ORF. By modulate is meant increasing, decreasing, or maintaining the level of activity of a particular element. The position of each regulatory element in the URE relative to each other and/or other elements may be expressed in terms of the 5′ terminus and the 3′ terminus of each element, and the distance between any particular regulatory elements may be referenced by the number of intervening nucleotides, or base pairs, between the elements. In some embodiments, the regulatory or enhancing effect of the URE is independent of positioning of the one or more regulatory elements in the URE. In some embodiments, the regulatory or transcription enhancing effect of the URE is dependent on its positioning and orientation with respect to the one or more regulatory elements in the URE.

The term “regulatory element” refers to a nucleic acid sequence which functions alone or in combination with other regulatory elements to regulate the expression of a gene. Exemplary regulatory elements include, without limitation, a promoter, a transcription factor binding site, an enhancer, a silencer, a boundary control element, an insulator, a locus control region, a response element, a binding site, a segment of a terminal repeat, a responsive site, a stabilizing element, a de-stabilizing element, a splicing element, a cis- or trans-regulatory element, a trans-activator, an inducible element, and a repressible element. Such regulatory elements are, in general, but not without exceptions, located 5′ to the coding sequence of the gene it controls, in an intron, or 3′ to the coding sequence of a gene, either in the untranslated or untranscribed region. As used herein, “strength of a unique regulatory element” refers to the amount of mRNA expression of, e.g., an ORF resulting from the unique regulatory element being operatively connected to the ORF in the context of, e.g., an expression vector, plasmid, or viral vector. As used herein, a “discrete regulatory element (DRE)” refers to a single, separate regulatory element. A DRE can be the same or different as another DRE within a combination in a URE.

As used herein, “Cis-regulatory element” or “CRE”, as used herein, is a term known to the skilled person as it relates to a regulatory element, and refers to a regulatory element which regulates the transcription of a transcribable reporter sequence that is on the same nucleic acid sequence. Cis-regulatory elements does not include proteins. A cis-acting regulatory element can be located 1500 nucleotides or less from the transcription start site (TSS), more preferably 1000 nucleotides or less from the TSS, more preferably 500 nucleotides or less from the TSS, and suitably 250, 200, 150, or 100 nucleotides or less from the TSS. As used herein, “Cis-regulatory module” or “CRM” refers to a is a stretch of DNA, for example, a stretch of 100-1000 base pairs in which at least 2, 3, 4, 5, or more CREs, e.g., a combination of CREs, bind and regulate expression of nearby genes, and/or regulate their transcription rates.

As used herein, “trans-regulatory element” or “TRE”, as used herein, is a term known to the skilled person as it relates to a regulatory element, and refers to a regulatory element which regulates the transcription of a transcribable reporter sequence that can be on a different nucleic acid construct. Trans-regulatory elements include proteins that interact with, e.g., bind to, a nucleic acid. For example, the tat protein and the TAR stem interaction resulting in trans-activation. A trans-acting regulatory element can be located on a distinct vector or synthetic nucleic acid construct that does not comprise a transcription start site (TSS) of the gene which it regulates.

As used herein, “discontinuous discrete regulatory element” or “dcDRE” refers to a discrete regulatory element that comprises at least two portions, that separately, do not comprise the function of a regulatory element. However, when the at least two portions of the dcDRE undergo a conformational change, e.g., that bring the at least two portions close proximity or in direct contact, they function as a regulatory element. Alternatively, the at least two portions of the dcDRE can comprise the function of a regulatory element separately, and have an increased function when having undergone a conformational change.

As used herein, the phrase “transcription factor target sequence” or “TFTS” or “transcription factor binding site” or “TFBS” or “TFBS motif” or “TFBM” refers to a region of DNA that generally contains specific sequences that are recognized and bound by transcription factors. Transcription factors bind to the TFBS and result in the recruitment of RNA polymerase, an enzyme that synthesizes RNA from the coding region of the gene.

As used herein, the phrase “promoter” refers to a region of DNA that generally is located upstream of a nucleic acid sequence to be transcribed that is needed for transcription to occur. Promoters permit the proper activation or repression of transcription of sequence under their control. A promoter typically contains specific sequences that are recognized and bound by transcription factors, e.g., enhancer sequences. Transcription factors bind to the promoter DNA sequences and result in the recruitment of RNA polymerase, an enzyme that synthesizes RNA from the coding region of the gene. A great many promoters are known in the art.

As used herein, “minimal promoter” refers to a short DNA segment which is inactive or largely inactive by itself, but can mediate strong transcription when combined with other transcription regulatory elements or the URE as defined herein. Minimal promoter sequence can be derived from various different sources, including prokaryotic and eukaryotic genes. Nonlimiting examples of minimal promoters are dopamine beta-hydroxylase gene minimum promoter and cytomegalovirus (CMV) immediate early gene minimum promoter (CMV-MP) and the herpes thymidine kinase minimal promoter (MinTK).

As used herein, “open reading frame”, refers to a sequence of nucleotides that, when read in a particular frame, do not contain any stop codons over the stretch of the open reading frame.

As used herein, “RNA transcript” or “transcript” refers to the product resulting from RNA polymerase-catalyzed transcription of a DNA sequence. When properly transcribed, a RNA transcript is typically an exact complementary copy of the DNA sequence, and is referred to as the primary transcript or it may be a RNA sequence derived from post-transcriptional processing of the primary transcript and is referred to as the mature RNA.

As used herein, “messenger RNA” or “(mRNA)” refers to the processed form of the transcript RNA that is without introns and that can be translated into protein by the cell.

As used herein, “barcode” refers to a short sequence of nucleotides (e.g., fewer than 40, 30, 25, 20, 15, 13, 12, or fewer nucleotides) included in a synthetic nucleic acid that can be transcribed into a transcript, e.g., an mRNA transcript, and is unique to a particular URE. The URE is comprised in plasmid, expression vector, or viral vector (exclusive of the region encoding the nucleic acid tag), and/or a short sequence of nucleotides included in a synthetic nucleic acid that are unique to the synthetic nucleic acid (exclusive of the region encoding the nucleic acid tag). A “plurality of barcodes” refers to at least two or more (e.g., at least 2, at least 3, at least 4, at least 5, at least 6, at least 7, at least 8, at least 9, at least 10, or more) unique barcodes in an undivided sample. A barcode “associated with a synthetic nucleic acid containing a URE” refers to a barcode included on an mRNA sequence (or cDNA derived therefrom) that was generated under the control of the particular URE. Because a barcode is “associated” with a particular URE, it is possible to determine the plasmid, expression vector, or viral vector (and, therefore, the URE located on the identified plasmid, expression vector, or viral vector) from which the barcoded mRNA (or cDNA derived therefrom) was generated.

As used herein, the term “operably linked” refers to an arrangement of elements wherein the components so described are configured so as to perform their usual function. For example, a given regulatory element operably linked to a transcribable reporter sequence, e.g., an ORF, e.g., a nucleic acid sequence with a coding sequence is capable of effecting the expression of that sequence when the proper enzymes are present. The URE as disclosed herein need not be contiguous with the sequence, so long as it functions to direct the expression of the gene encoded by the ORF. Thus, for example, intervening untranslated yet transcribed sequences can be present between the URE and the ORF and the URE or regulatory element sequence can still be considered “operably linked” to a ORF or nucleic acid with a coding sequence. Thus, the term “operably linked” is intended to encompass any spacing or orientation of the regulatory element and the ORF or coding sequence of interest which allows for initiation of transcription of the coding sequence of interest upon recognition of the URE by a transcription complex. As understood by the skilled person, operably linked implies functional activity, and is not necessarily related to a natural positional link. Indeed, when used in nucleic acid expression cassettes, cis-regulatory elements are located on the same nucleic acid construct as the ORF and can, in some embodiments be located immediately upstream of the ORF or minimal promoter, or alternatively downstream of the gene in the ORF (although this is generally the case, it should definitely not be interpreted as a limitation or exclusion of positions within the nucleic acid expression cassette). Alternatively, a trans-regulatory elements are located on a different nucleic acid construct as the ORF and can still be operatively linked to the ORF. When trans-regulatory elements are referenced, it meant to indicate that the trans element, or other elements therein, are altered.

The term “vector,” as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, “expression vector” refers to a nucleic acid that includes a transcribable reporter sequence, e.g., ORF, and, when introduced to a cell, contains all of the nucleic acid components necessary to allow mRNA expression of said open reading frame. “Expression vectors” of the invention also include elements necessary for replication and propagation of the vector in a host cell. In particular, as used herein, “expression vector” refers to a vector that directs expression of a synthetic nucleic acid described herein. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing.

As used herein, “conformation” refers to the overall three-dimensional structure of a construct under a given set of conditions. In one embodiment, a model conformation is the conformation of the wild type (unaltered) sequence under the normal conditions the construct would encounter in vivo such as physiological non-reducing conditions.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring synthetic nucleic acids described herein into cells either in vitro or in vivo. Numerous forms of viral vectors are known in the art.

As used herein, the term “expression” refers to the cellular processes involved in producing RNA and proteins, including where applicable, but not limited to, for example, transcription, transcript processing, translation and protein folding, modification and processing.

The term “expression products” include RNA transcribed from a gene, and polypeptides obtained by translation of mRNA transcribed from a gene.

The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “cell culture”, as used herein, refers to a proliferating mass of cells that may be in either an undifferentiated or differentiated state.

As used herein, “introducing” refers broadly to placing the synthetic nucleic acid, expression vector, or plasmid into a host system (e.g., a cell or viral vector) such that it is present in the host system. Less broadly, introducing refers to any appropriate means of placing the synthetic nucleic acid, expression vector, or plasmid in a host system described herein. Introducing can be by such means that the synthetic nucleic acid, expression vector, or plasmid is appropriately transported into the interior of the host system such that, e.g., the synthetic nucleic acid, expression vector, or plasmid is produced by the host cell machinery. Such introducing may involve, for example transformation, transfection, electroporation, or lipofection.

As used herein, “determining the expression frequency” refers to determining of the relative abundance of a particular barcode produced in a cell (output) as normalized to each barcode content (input) before expression in the cell.

The term “consensus sequence” follows the meaning of consensus sequence is well-known in the art. In the present application, the following notation is used for the consensus sequences, unless the context dictates otherwise. Considering the following exemplary DNA sequence: A[CT]N{A}YR. In this instance, A means that an A is always found in that position; [CT] stands for either C or T in that position; N stands for any base in that position; and {A} means any base except A is found in that position. Y represents any pyrimidine, and R indicates any purine.

The terms “identity” and “identical” and the like refer to the sequence similarity between two polymeric molecules, e.g., between two nucleic acid molecules, e.g., two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5: 151-3; Corpet et al. (1988) Nucleic Acids Res. 16: 10881-90; Huang et al. (1992) Comp. Appl. Biosci. 8: 155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence. For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, +10%, +5%, +1%, +0.5%, or even ±0.1% of the specified amount.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

I. Synthetic Nucleic Acids

We have found that substantial errors can arise if the synthetic nucleic acid and portions thereof do not satisfy certain criteria. Aspects of this invention relate to a plurality of synthetic nucleic acids comprising (1) a first plurality of synthetic nucleic acids each comprising a unique regulatory element (URE) where the URE comprises (i) a nucleic acid sequence containing at least one discrete regulatory element (DRE), wherein the DRE is a control (or wild type) continuous nucleic acid sequence or a control discontinuous nucleic acid sequence associated with a plurality of unique barcodes corresponding with the at least one DRE, wherein each barcode is between 12-35 nucleotides in length and has a GC content between 25-65%; and (ii) the DRE is conformationally positioned in a preselected manner relative to a nucleic acid encoding a transcribable reporter sequence, e.g., ORF, wherein if the URE does not contain a promoter, a separate promoter is operatively linked to the transcribable reporter sequence; and (2) a second plurality of synthetic nucleic acids comprising a URE that further comprises a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%.

Another aspect of the invention is a plurality of synthetic nucleic acids comprising at (1) a first plurality of synthetic nucleic acids each comprising a unique regulatory element (URE), wherein the URE comprises (i) a nucleic acid sequence containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is conformationally positioned in a preselected manner relative to a nucleic acid encoding a transcribable reporter sequence operatively linked to a promoter; wherein if the URE does not contain a promoter, a separate promoter is operatively linked to the transcribable reporter sequence; and (2) a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%.

Another aspect of the invention is a plurality of synthetic nucleic acids comprising at (1) a unique regulatory element (URE), wherein the URE comprises (i) a first plurality of synthetic nucleic acid sequences each containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is positioned in a preselected manner relative to a nucleic acid encoding a transcribable reporter sequence, e.g., ORF, operatively linked to a promoter; wherein if the URE does not contain a promoter, a separate promoter is operatively linked to the transcribable reporter sequence; and (2) a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%.

Elements of a synthetic nucleic acid described herein, e.g., at least one URE comprising a combination of DREs, a TR or partial TR, at least one transcribable reporter sequence, e.g., ORF, and a plurality of barcodes, may be arranged in a variety of configurations. For example, the at least one plurality of barcodes may be located anywhere within the region to be transcribed into mRNA (e.g., upstream of the transcribable reporter sequence, downstream of the transcribable reporter sequence, or within the transcribable reporter sequence). Importantly, the barcode is to be located 5′ to the transcription termination site.

In one embodiment, the plurality of synthetic nucleic acids comprises at least 50 synthetic nucleic acids. In another embodiment, the plurality of synthetic nucleic acids comprises at least 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, or more synthetic nucleic acids.

The length of a heterologous nucleic acid sequence directly effects the efficiency in which it is properly integrated into a viral vector, for example, an AAV vector; shorter sequences have been shown to be integrated less efficiently as compared to a longer sequence. In one embodiment, the synthetic nucleic acid backbone further comprises at least 350 bp to 650 bp of additional nucleotide sequence for expression in a viral vector. In another embodiment, the synthetic nucleic acid further comprises at least 50 bp, 100 bp, 150 bp, 200 bp, 250 bp, 300 bp, 400 bp, 450 bp, 500 bp, 600 bp, 650 bp, 700 bp, 750 bp, 800 bp, 850 bp, 900 bp, 950 bp, 1000 bp, or more of additional nucleotide sequence for expression in a viral vector. The additional sequence can be a non-functional sequence (e.g., a sequence that creates length within the synthetic nucleic acid, or space between the components of the synthetic nucleic acid but does not itself contribute any sequence specific effect on the synthetic nucleic acid's activity). In one embodiment, the at least 350 bp to 650 bp of additional nucleotide sequence functions to avoid the presence of regulatory elements interfering with promoter activity. In one embodiment, the at least 350 bp to 650 bp of additional nucleotide sequence is a 565 bp long internal antisense out-of-frame fragment from the Blitzen-Blue reporter gene specific for Pichia pastoris. In one embodiment, the at least 350 bp to 650 bp of additional nucleotide sequence is integrated in the 3′ end of the AAV screening cassette.

Synthetic nucleic acids described herein are generated by any means known in the art, including through the use of polymerases and solid state nucleic acid synthesis (e.g., on a column, multiwall plate, or microarray). Furthermore, a plurality of nucleic acid constructs may be generated by first generating a parent population of constructs (e.g., as described above) and then diversifying the parent constructs (e.g., through a process by which parent nucleotides are substituted, inserted, or deleted) resulting in a diverse population of new nucleic acid constructs. The diversification process may take place, e.g., within an isolated population of nucleic acid constructs with the nucleic acid regulatory element and tag in the context of an expression vector, where the expression vector also contains an ORF operatively connected to the nucleic acid regulatory element.

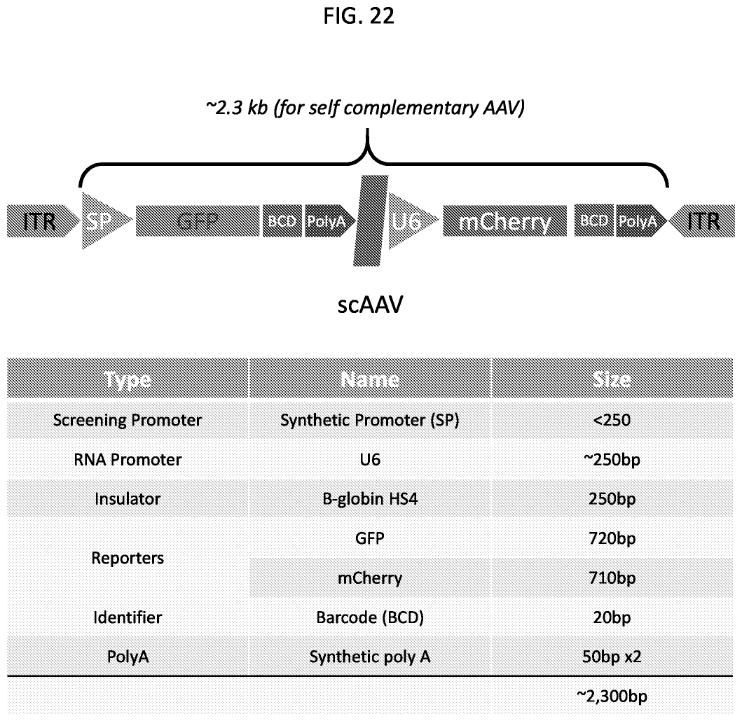

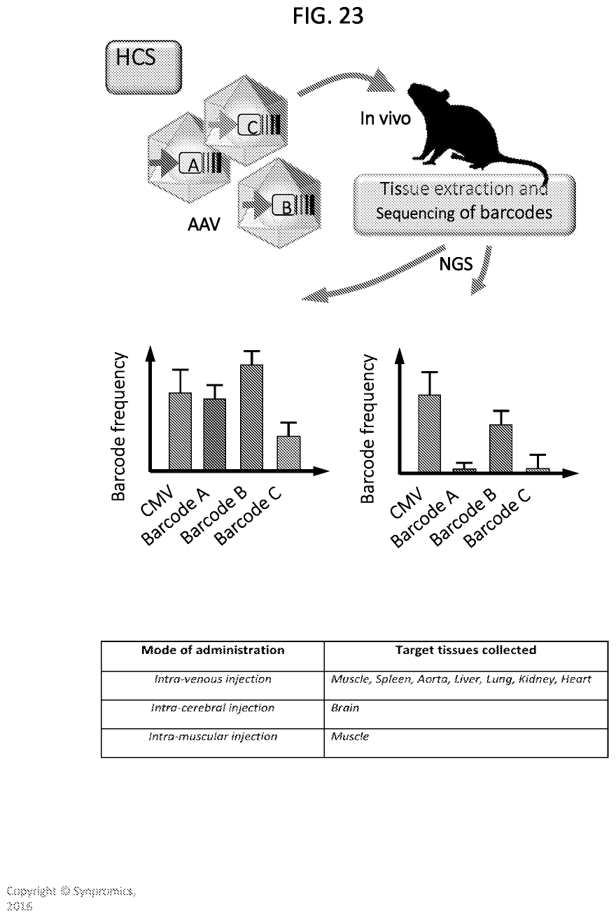

In one embodiment, the synthetic nucleic acid further comprises a second reporter gene. In one embodiment, the second reporter gene is a low level reporter gene which is used to normalize expression of the plurality of synthetic nucleic acid in the cell, or population thereof (see e.g., FIG. 22). In one embodiment, the second reporter gene is located in an insulator sequence, e.g., β-globin H4S sequence.

In one embodiment, the second reporter gene allows for multiplexed therapeutic synthetic nucleic acid screenings in the context of a vector, for example an AAV vector, with a normalizer expressed from within each individual AAV/expression cassette combination. In one embodiment, two barcoded synthetic nucleic acids (e.g., expression cassettes) are packaged into a vector, e.g., an AAV vector; the first synthetic nucleic acid is driven by the promoter of interest, and the second synthetic nucleic acid by a weak constitutive promoter. The barcodes of each synthetic nucleic acid promoter and normalizer are linked. Each synthetic nucleic acid contains one of two fluorescent proteins, e.g., green fluorescent protein, cherry fluorescent protein, yellow fluorescent protein, or the like. The effective strength of each synthetic nucleic acid is determined by the barcode:normalizer ratio. In one embodiment, methods using the second, low level reporter gene allow for the cells to be sorted based on 1) the amount of fluorescent protein, and/or 2) the amount of normalizer protein to bias for active promoters in widely diffused or highly concentrated AAV expression.

II. Unique Regulatory Element (URE)

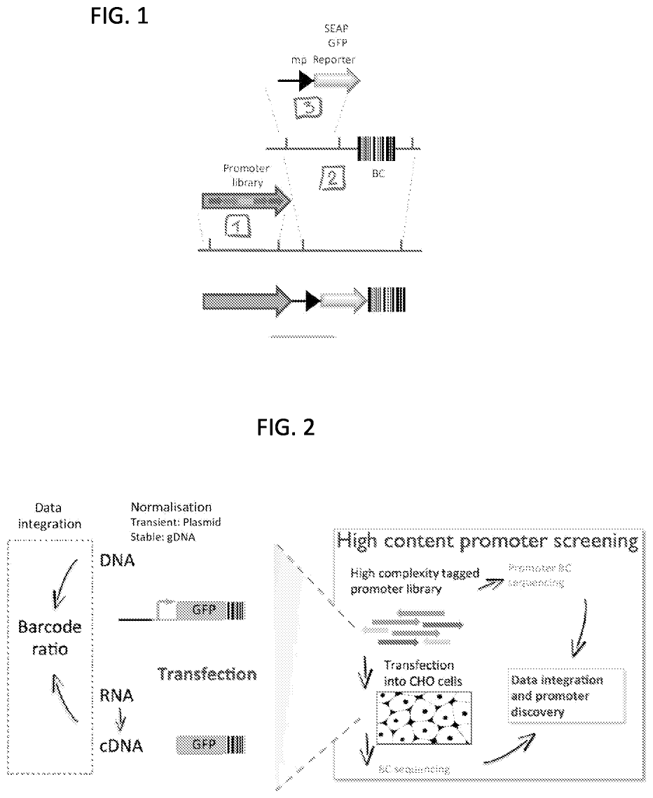

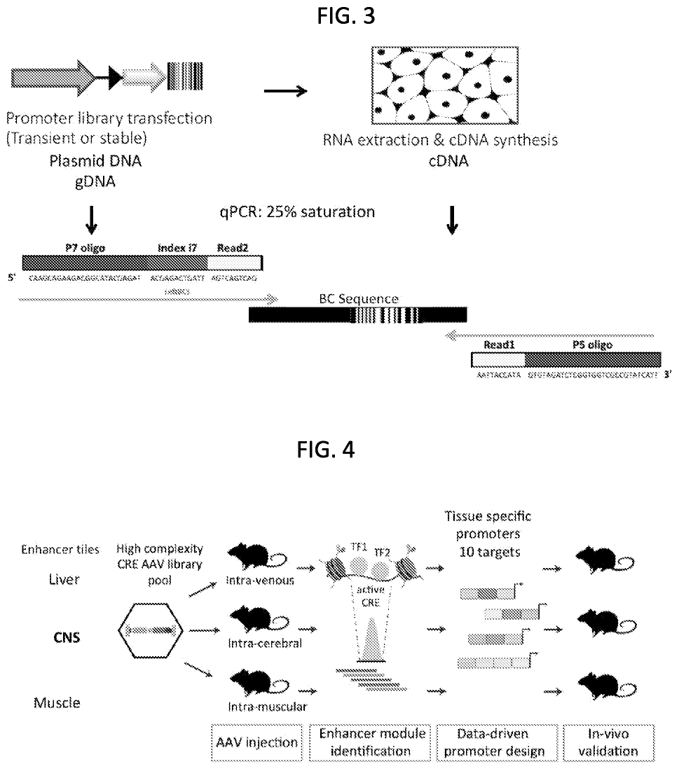

A suitable URE for use in the synthetic nucleic acids described herein is one that is active in the cell or tissue of interest. A URE has at least one discrete regulatory sequence (DRE) present. For example, the URE can have multiple regulatory elements in a unique combination or in unique spacing or both. These regulatory elements include, e.g., a transcription factor binding site, a cis- or trans-regulatory element, an enhancer, a silencer, a boundary control element, an insulator, a locus control region, a response element, a binding site, a trans-activator, a responsive site, a stabilizing element, a de-stabilizing element, a splicing element, an inducible element, a repressible element, a promoter, a segment of a terminal repeat, etc. The URE can be comprised of these regulatory elements in various combinations or orientations. Barcodes should preferably be attached to each regulatory element for precision in defining and determining the strength of the combination and orientation of different regulatory elements. In one embodiment, UREs are non-arbitrarily identified, i.e., via a bioinformatics approach in which, e.g., a cell type is profiled to identify highly expressed genes. One skilled in the art can assess the gene profile of, e.g., a specific cell type, using standard techniques, for example quantitative PCR, serial analysis of gene expression (SAGE), or microarray analysis. Next, UREs comprising a pool of TFBS or CREs, (for example, as described herein below in Examples) associated with these highly expressed genes are identified, weighted and ranked. A library of top weighted/ranked UREs are assembled by synthesizing a “DNA fragment” comprising the TFBSs. Compatible restriction sites, e.g., (Nhe1) and (AvrII and XbaI), are used for purification of the DNA fragment harbouring individual or a pool of TFBSs. The DNA fragment comprising TFBSs is further ligated with specific adapters for performing in-fusion PCR for vector integration. The DNA fragment thus ligated to adapters are referred to as UREs or the synthetic promoter constructs as described herein below in the Examples. The orientation of the reannealed URE within the synthetic nucleic acid is random, e.g., a URE can reanneal from 5′ to 3′, or 3′ to 5′. Using standard cloning techniques, additional components of the synthetic nucleic acid, e.g., a transcribable reporter sequence, such as an ORF and a plurality of barcodes are added to make the URE. FIG. 2 herein shows exemplary strategy to generate the synthetic nucleic acids as disclosed herein, i.e., to integrate the URE with the open reading frame and barcode. FIG. 1 shows an exemplary example of generating a URE comprising multiple transcription factor target sites (TFTS).

In another embodiment, a URE is selected based on its association with a differentially expressed gene, e.g., a gene that is differentially expressed in that cell, tissue, or condition, when compared with another cell, tissue or condition. For example, differential expression of a gene may be seen by comparing the gene profile in two different cells, tissues, or conditions, and/or in the same cells or tissues under different conditions. Expression in one cell or tissue type may be compared with that in a different, but related, tissue type. For example, where the cell or tissue of interest is a disease cell or tissue, the expression of genes in that cell or tissue may be compared with the expression of the same genes in an equivalent normal (e.g., healthy) cell or tissue. In one embodiment, UREs from multiple differentially expressed genes are used in combination, e.g., to create a unique combination of regulatory elements.

In another embodiment, UREs are selected arbitrarily, i.e., at random. Methods for designing synthetic promoters for eukaryotic systems that involve the arbitrary selection of well-characterized UREs, e.g., cis-regulatory elements, spanning 50 to 100 nucleotides have been described. As disclosed herein, the UREs could be between 50-800 bp or between 250-600 bp. Such UREs then are included in synthetic promoter libraries created by random ligation and selected for in the cell type of interest (Li, X., Eastman, E. M., Schwartz, R. J., & Draghia-Akli, R. Synthetic muscle promoters: activities exceeding naturally occurring regulatory sequences. Nat. Biotechnol. 17, 241-245 (1999); Dai, C., McAninch, R. E., & Sutton, R. E. Identification of synthetic endothelial cell-specific promoters by use of a high-throughput screen. J. Virol. 78, 6209-6221 (2004)), the contents of each of which are incorporated herein by reference in their entireties.

In one embodiment the regulatory element, sometimes referred to as the DRE, is a promoter, a transcription factor binding site, an enhancer, a silencer, a boundary control element, an insulator, a locus control region, a response element, a binding site, a segment of a terminal repeat, a responsive site, a stabilizing element, a de-stabilizing element, or a splicing element. In one embodiment, the promoter can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. These are all parts of the URE.

The DRE or regulatory element comprised in a URE may be naturally-occurring sequences, variants based on the naturally-occurring sequences, or wholly synthetic sequences. The source of the URE is not critical, however, in one embodiment, it is preferred that a URE is assessed in the environment from which it is derived (e.g., the strength of a liver promoter should be assessed in a liver cell in vitro or within the liver in vivo). Variants include those developed by single (or greater) nucleotide scanning mutagenesis (e.g., resulting in a population of UREs containing single mutations at each nucleotide contained in the naturally-occurring regulatory element), transpositions, transversions, insertions, deletions, or any combination thereof. UREs may include non-functional sequences (e.g., sequences that create space between the at least two UREs but do not themselves contribute any sequence specific effect on the URE's activity). When referring to a CRE that does not itself comprise a regulatory function (e.g., does not itself modulate the activity of a transcribable reporter sequence), it is understood that this is in reference to a region that contains groupings of CREs, CRMs, and/or regulatory elements in which the spacing can be altered to optimize their function. Comparisons and alterations are made with respect to such groupings.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268: 1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

A synthetic nucleic acid can have more than one DRE, i.e., a combination of DREs. For example, in one embodiment, the synthetic nucleic acid has at least 2, 3, 4, 5, 6, 7, 8, 9, 10, or more DREs. The multiple DREs can be directly up or down stream of each other, or separated by several base pairs. Where a synthetic nucleic acid has more than three DREs, the DREs can be directly up or downstream of each other and separated by several base pairs. In one embodiment, the at least 2, 3, 4, 5, 6, 7, 8, 9, 10, or more DREs, or combination of DREs, are associated with the same plurality of unique barcodes. In one embodiment, the plurality of barcodes are preferably less than 12 and more suitably less than 10.

In one embodiment, the at least one DRE and transcribable reporter sequence, e.g., ORF, are separated by at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs. In another embodiment, the combination of DRE comprises at least two DRE and the at least two DRE are separated by at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs. The intervening sequence (e.g., the at least 2 base pairs positioned in between the DRE and the ORF or the at least two DREs) can comprise any sequence and can be assigned at random. It is desired that the intervening sequence does not interfere with the sequence of the synthetic nucleic acid, e.g., does not affect the structure, expression, folding, etc. of the synthetic nucleic acid. Ideally, the intervening sequence is a scrambled sequence, e.g., a randomized sequence that does not translate a protein, or alternatively is a known linker sequence. Using such spacing differences, the present method can be used to determine the effect of spacing these components on the strength of expression.

In one embodiment, the at least one URE and the TR sequence are separated by 1-500 base pairs. In one embodiment, the at least one URE and the TR sequence are separated by at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs. In another embodiment, the at least one URE and the at least partial TR sequence are separated by 1-500 base pairs. In one embodiment, the at least one URE and the at least partial TR sequence are separated by at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs. While such distances are large linearly, the sequences may be relatively near each other when looked at in their 3-dimensional conformation. The intervening sequence (e.g., the at least 2 base pairs positioned in between the URE and the TR) can comprise any sequence and can be assigned at random. It is desired that the intervening sequence does not interfere with the sequence of the URE or TR, or portion thereof, e.g., does not affect the structure, expression, folding, etc. of the URE or TR, or portion thereof. Ideally, the intervening sequence is a scrambled sequence, e.g., a randomized sequence that does not translate a protein, or alternatively is a known linker sequence. Using such spacing differences, the present method can be used to determine the effect of spacing these components on the strength of expression. One can use linker substitutions to maintain conformation.

In some embodiments, a URE comprises at least one regulatory element, or comprises two or more, preferably three or more, suitably five or more, copies of at least one regulatory element. In some embodiments, the regulatory element can be a transcription factor target sequence, as disclosed herein. In one embodiment, a URE comprises at least one TFBS or comprises two or more, preferably three or more, suitably five or more, TFBS. In some embodiments, a regulatory element is selected from any of, but is not limited to, a promoter, a mini-promoter, a riboswitch, an insulator, a mir-regulatable element, a post-transcriptional regulatory element, a tissue- and cell type-specific promoter and an enhancer. In some embodiments, a regulatory element can comprise an ITR, or part of a ITR.

In some embodiments, a URE can comprise regulatory element isolated from any other prokaryotic, viral, or eukaryotic cell; and synthetic regulatory element, e.g., regulatory elements that are not “naturally occurring,” i.e., comprise different sequences or mutations of the endogenous regulatory element. In some embodiments, the regulatory element can be modified through methods of genetic engineering that are known in the art. In addition, regulatory elements can be synthetic regulatory elements produced using recombinant cloning and/or nucleic acid amplification technology, including PCR (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as regulatory elements in the URE as well.

In some embodiments, the URE is a synthetic sequence. In some embodiments, the URE comprises one or more DRE or transcription factor target sequences. In some embodiments, the regulatory element or TF target sequences may be directly adjacent to each other (e.g., in tandem, or tandem repeats) or may be spaced apart. In some embodiments, the regulatory element or TF target sequences can function in cis- or in trans. For example, a regulatory element that functions in cis- with another regulatory element are regulatory elements that are present on the same nucleic acid construct. That is, the regulatory element's functioning in cis- can be adjacent to each other, or spatially separated, yet on the same nucleic acid construct. For example, the regulatory element that functions in cis- can, for example, be located as much as several thousand base pairs from the other regulatory element, or the start site of transcription.

Alternatively, a DRE that functions in trans- with another regulatory element is where the regulatory elements are present on distinct (or separate) nucleic acid constructs. In some embodiments, a regulatory element that functions in trans- with another regulatory element can have enhanced function when it is in cis- with the corresponding regulatory element.

As disclosed herein, a URE can comprise a combination of DREs. A DRE can comprise a portion or fragment of a promoter. In some embodiments, a URE can comprise one or more specific regulatory element sequences to further enhance expression and/or to alter the spatial expression and/or temporal expression of same. A URE can also comprise any one or more of enhancer or repressor elements, which may be located as much as several thousand to over a million base pairs from the start site of transcription in the genome. A regulatory element may be derived from sources including viral, bacterial, fungal, plants, insects, and animals. An URE may regulate the expression of a gene constitutively, or differentially with respect to the cell, tissue or organ in which expression occurs or, with respect to the developmental stage at which expression occurs, or in response to external stimuli such as physiological stresses, pathogens, metal ions, or inducing agents.

A URE can comprise a range of DRE, for example, DREs that can be modulated by small molecule switches or inducible or repressible promoters. Non-limiting examples of regulatory elements include TF target sequences for hormone-inducible or metal-inducible genes.

The term “regulatory element” as used herein refers a cis- or trans-acting regulatory sequence (e.g., 50-1,500 base pairs) that bind one or more proteins (e.g., activator proteins, or transcription factor) to modulate (e.g., increase or decrease) transcriptional activation of a nucleic acid sequence. In some embodiments, a regulatory element can be positioned up to 1,000,000 base pars upstream of the gene start site, or downstream of the gene start site that they regulate, e.g., in an endogenous genome. In some embodiments, a regulatory element can be positioned within an intronic region, or in the exonic region of an unrelated gene.

A URE as disclosed herein can be said to drive expression or drive transcription of the nucleic acid sequence that it regulates. The phrases “operably linked,” “operatively positioned,” “operatively linked,” “under control,” and “under transcriptional control” indicate that a URE is in a correct functional location and/or orientation in relation to a nucleic acid sequence it regulates to control transcriptional initiation and/or expression of that sequence.

An “inverted” used to define the orientation of a regulatory element or TF target sequence, as used herein, refers to a regulatory element in which the nucleic acid sequence is in the reverse orientation, such that what was the sense strand is now the antisense strand, and vice versa. In some embodiment, an inverted regulatory element sequence is in the reverse orientation as it exists in nature. Inverted regulatory element sequences can be used in various embodiments in a URE.

In some embodiments, a URE comprises at least two regulatory element sequences, where the regulatory element sequences are separated by a spacer sequence or another functional sequence (e.g. another regulatory element or TF target sequence). In some embodiments, a spacer sequence, if present, is from 5-50 nucleotides in length, but it can be longer or shorter in some cases. For example, the spacer sequence is suitably from 2 to 50 nucleotides in length, suitably from 4 to 30 nucleotides in length, or suitably from 5 to 20 nucleotides in length. In some embodiments, the spacer sequence is a multiple of 5 nucleotides in length, as this provides an integer number of half-turns of the DNA double helix (a full turn corresponding to approximately 10 nucleotides in chromatin). A spacer sequence length that is up to 10, or a multiple of 10 nucleotides in length may be more preferable, as it provides an integer number of full-turns of the DNA double helix. The spacer sequence can have essentially any sequence, provided it does not prevent the regulatory element or URE from functioning as desired (e.g. it includes a silencer sequence, prevents binding of the desired transcription factor, or suchlike). The spacer sequences between each regulatory element, e.g., TF target sequence can be identical or they can be different.

In some embodiments, a regulatory element is TF target sequence. An exemplary TF target sequence comprises one or more copies of the transcription factor target sequence TGACGTG (SEQ ID NO: 4) (i.e. the ATF6 consensus sequence). In one embodiments, a URE comprises preferably 3 or more copies of the TF target sequence, and preferably 5 or more copies of the TF target sequence, for example 6 or more copies of the a TF target sequence. For illustrative purposes only, using TGACGTG (SEQ ID NO: 4) as an exemplary TF target sequence, the URE comprises the transcription factor target sequence TGACGTG (SEQ ID NO: 4), and preferably 5 or more copies of the transcription factor target sequence TGACGTG (SEQ ID NO: 4), for example 6 or more copies of the transcription factor target sequence TGACGTG (SEQ ID NO: 4). In some embodiments, a URE comprises preferably 3 or more TFBSs, and preferably 5 or more TFBSs, for example 6 or more TFBSs. In some embodiments, a URE can comprise TF target sequences as a tandem repeat or they may be spaced from each other. Generally, in some embodiments, at least two, and preferably all, of the regulatory element sequences, e.g., TF target sequence present in the URE are spaced from each other, e.g. by a spacer sequence as discussed above.

Again, for illustrative purposes only, using TGACGTG (SEQ ID NO: 4) as an exemplary TF target sequence, in some embodiments, a URE comprises one or more copies of the transcription factor target sequence TGACGTG (SEQ ID NO: 4), preferably 3 or more copies of the transcription factor target sequence TGACGTG (SEQ ID NO: 4), preferably 5 or more copies of the transcription factor target sequence TGACGTG (SEQ ID NO: 4), for example 6 or more copies of the transcription factor target sequence TGACGTG (SEQ ID NO: 4). As mentioned above, these regulatory element sequences, e.g., TF target sequences, may be in tandem repeat, or may be spaced from each other. Generally, in some embodiments, at least two, and preferably all, of regulatory element sequences, e.g., TF target sequence present in the URE are spaced from each other, e.g. by a spacer sequence as discussed above. In some embodiments, a regulatory element sequence, e.g., TF target sequence TGACGTGCT (SEQ ID NO: 1) has been found to be particularly effective when used in multiple copy number in a URE, whether as a tandem repeat or including spacer sequences.

In some embodiments, the URE comprises regulatory element sequences, e.g., TF target sequence (represented by “TFTS”) separated by spacers, for example, TFTS-S-TFTS-S-TFTS-S-TFTS-S-TFTS-S-TFTS, where S represents an optional spacer sequence as defined above. In some embodiments, spacer sequences are present between at least two, and preferably all, of the regulatory element sequences, e.g., TF target sequence. For example, continuing with TGACGTG (SEQ ID NO: 4) as an exemplary TF target sequence, in some embodiments, the URE comprises regulatory element sequences, e.g., TF target sequence TGACGTG-S-TGACGTG-S-TGACGTG-S-TGACGTG-S-TGACGTG-S-TGACGTG (“TGACGTG” disclosed as SEQ ID NO: 2), where S represents an optional spacer sequence as defined above. In some embodiments, spacer sequences are present between at least two, and preferably all, of the regulatory element sequences, e.g., TF target sequence (TGACGTG (SEQ ID NO: 4)).

In some embodiments, an exemplary spacer has the following sequence: GATGATGCGTAGCTAGTAGT (SEQ ID NO: 3), or a sequence that is at least 50% identical thereto, or at least 70% identical thereto, or at least 80% identical thereto, or at least 85%, 90%, 995%, 98% or 99% identical thereto. In some embodiments, sequence variation only occurs in sequences which are not the TF target sequences. In some embodiments, sequence variation only occurs in spacer sequences.

In some embodiments, if the URE does not contain a promoter, a separate promoter is operatively linked to the transcribable reporter sequence, e.g., ORF. In one embodiment, the separate promoter is operatively linked to the ORF is a minimal promoter (MP). In some embodiments, a minimal promoter is a CMV-MP minimal promoter. Other minimal promoters known in the art are envisioned for use, including but not limited to the herpes thymidine kinase minimal promoter (MinTK), Sv40 mp, and YB TATA mp. It is highly preferred that sequence variation only occurs in sequences which are not the transcription factor target sequences, i.e. those having the sequence TGACGTG (SEQ ID NO: 4), nor in the CMV-MP sequence. The CMV-minimal promoter has the following sequence: AGGTCTATATAAGCAGAGCTCGTTTAGTGAACCGTCAGATCGCCTAGATACGCC ATCCACGCTGTTTT GACCTCCATAGAAGAT (SEQ ID NO: 5). The MinTK promoter has the following sequence: GCAGTTAGCGTAGCTGAGGTACCGTCGACGATATCGGATCCTTCGCATATTAAG GTGACGCGTGTGGCCTCGAACACCGAG (SEQ ID NO: 6). In some embodiments, the URE is operatively linked to a minimal promoter of having the CMV-MP sequence, or the MinTK sequence, or a sequence that is at least 50% identical thereto, or at least 70% identical thereto, or at least 80% identical thereto, or at least 85%, 90%, 995%, 98% or 99% identical thereto. Accordingly, in some embodiments, the URE is operably linked to the CMV-MP minimal promoter, or the MinTK minimal promoter.

In an alternative embodiment, the transcribable reporter sequence is not necessary.

In some embodiments, the minimal promoter preferably does not drive transcription of an operably linked gene when present in a eukaryotic cell in the absence of the URE. The URE drives transcription of an operably linked gene when present in a eukaryotic cell when the URE is occurring in the cell. Assessment of the ability of a URE to selectively drive transcription can readily be assessed by the skilled person using a wide range of approaches, and these can be tailored for the particular expression system in which the construct is intended to be used. As one preferred example, the methodology described in the Examples below can be used, e.g., as described herein in Example 1. For example, any candidate URE to be assessed can be substituted into the construct described in Example 1 in place of the exemplary URE used in Example 1, and the ability of said candidate URE to selectively drive transcription when the URE is induced can be measured by assessing the level of the reporter gene, e.g., GFP expression or luciferase expression before and after URE induction as carried out in Example 1. A URE is one which is able to be successfully induced to significantly increase transcription of an operably linked gene (in the case of Example 1, the luciferase gene) upon induction of the URE to result in the expression of the gene.

UREs associated with a given gene are generally located near, but not limited to, the coding sequence of the gene within the genome of the cell. For example, a URE may be located in the region immediately upstream or downstream of that coding sequence. A URE may be located close to a promoter or other regulatory sequence region that regulates expression of the gene. The location of a URE may be determined by the skilled person using standard techniques, e.g., via searching available microarray and/or genome sequence, or genome sequence of the identified gene, looking for known chromosomal markers that indicate a URE. Microarray data and next generation sequence data, e.g., the complete human genome sequence, can be searched for potential UREs by, e.g., comparing the upstream non-coding regions of multiple genes that show similar expression profiles under certain conditions. Exemplary microarray data and complete human genome sequences can be found, e.g. in (Roth, F. P., Hughes, J. D., Estep, P. W., & Church, G. M. Finding DNA regulatory motifs within unaligned noncoding sequences clustered by whole-genome mRNA quantitation. Nat. Biotechnol. 16, 939-945 (1998)), from simple expression ratio (Bussemaker, H. J., Li, H., & Siggia, E. D. Regulatory element detection using correlation with expression. Nat. Genet. 27, 167-171 (2001)) or functional analysis of gene products (Jensen, L. J. & Knudsen, S. Automatic discovery of regulatory patterns in promoter regions based on whole cell expression data and functional annotation. Bioinformatics. 16, 326-333 (2000)). All references cited herein are incorporated by reference in their entireties.

The methodology and the components used permit the selection of UREs for a range of criteria. For example, one can identify various promoters and/or enhancers. After selection of a desired URE, e.g., a strong promoter, one can then screen the characteristics of that promoter in a range of cell types. One can then identify differences in the characteristics of that promoter based upon where it is placed relative to a gene, or relative to different genes. The desired system can be screened for differences in in vivo relative to in vitro performance.

In some embodiments, a URE confers at least a 2-fold increase in expression as compared to a known tissue specific promoter for the tissue type being assessed. In some embodiments, a URE confers at least a 2-fold, or at least 2.5-fold, or at least 5-fold, or at least 7.5 fold, or at least a 10-fold, or more than 10-fold increase in expression, more preferably at least a 100-fold increase in expression, and yet more preferably at least a 1000-fold increase in expression of the reporter gene (e.g. luciferase) as compared to the expression level of a known tissue specific promoter for the tissue type being assessed. It is preferred that before induction of the URE, the expression levels of the reporting gene (e.g., luciferase) are minimal, significantly less than that of induced expression, or preferably, negligible. Minimal expression can be defined as, for example, equal to or less than the expression levels of a control construct (CMV-MP or CMV IE MP alone), and is preferably less than 50%, preferably less than 20%, more preferably less than 10%, yet more preferably less than 5%, yet more preferably less than 1% of the induced expression levels. Negligible expression levels are, for example, those that are essentially undetectable using the methodology of Example 1 described herein below.

In one embodiment, at least one DRE is a discontinuous DRE (dcDRE).

In one embodiment, the synthetic nucleic acid contains at least 2, 3, 4, 5, 6, or more dcDREs.

In one embodiment, the at least one dcDRE comprises at least one modification, e.g., a nucleotide substitution, insertion, or deletion. In one embodiment, the at least one dcDRE comprises at least 2, 3, 4, 5, 6, or more modifications.

In one embodiment, each portion of a dcDRE is separated by 1-500 base pairs. In one embodiment, each portion of a dcDRE is separated by at least 50 base pairs. In one embodiment, each portion of a dcDRE is separated by at least 2, 3, 4, 5, 6, 7, 8, 9, 10, 11, 12, 13, 14, 15, 16, 17, 18, 19, 20, 21, 22, 23, 24, 25, 100, 150, 200, 250, 300, 350, 400, 450, 500, 550, 600, 650, 700, 750, 800, 850, 900, 950, 1000, 1500, 10000, 15000 or more base pairs. If a dcDRE comprises more than 2 portions, then the more than two portions can be separated by the same number of base pairs (e.g., a dcDRE having 3 portions equaling separated by 250 base pairs), or by different numbers of base pairs (e.g., a dcDRE having 3 portions in which the first two portions are separated by 350 base pairs, and the second two portions are separated by 700 base pairs). The spacing between portions of a dcDRE can be naturally occurring (e.g., as it naturally occurs in a wild-type sequence), or can be modulated to increase or decrease the space as it naturally occurs. The spacing between each portion of the dcDRE can contribute to the functionality of the dcDRE, e.g., the correct spacing allows for, e.g., a conformational change required for the dcDRE function.

In one embodiment, one portion of a dcDRE can be 5′ of the ORF, and a second portion of the dcDRE is 3′ of the ORE. In an alternate embodiment, at least one portion of the dcDRE is found within a ORF. In one embodiment, the dcDRE comprises a portion of the DRE located 5′ of the ORF, and a portion of the DRE located 3′ of the open reading frame.

In one embodiment, the dcDRE comprises a non-DRE nucleic acid sequence located in a 5′- or 3′-portion of the DRE.

In one embodiment, a URE is identified as being associated with a highly expressed gene, e.g., in a cell, a tissue, an organ. For example, a URE can be associated with a gene highly expressed in the live. Using meta-analysis of microarray data from liver cells obtained from various studies, e.g., Zhang, H., et al. Nutr Metab (Lond). 2016; 13: 63; Guillen, N., et al. Physiol Genomics. 2009 May 13; 37(3):187-98; and Yamazaki, K, et al. Biochemical and Biophysical Research Communications. January 2002; 290(3):1114-1122, highly expressed genes are identified. Genes identified as being highly expressed in the liver are ranked by their expression reported expression levels. Further, the literature is searched using pubmed in order to find if genes identified as being highly expressed in the liver were previously been shown by independent methods. Depending on the expression levels and assays used for detection, genes are scored as “+++”—Substantial evidence to support their overexpression; “++”—Significant evidence to support their overexpression, and “+”-Evidence to support their overexpression. Genes with no further evidence regarding their overexpression in the liver are excluded. Finally, the regulatory regions of the genes identified as being highly expressed in the liver are analyzed to identify potential cis-regulatory elements are examined. Potential cis-regulatory elements are cloned into a DNA-fragment. Compatible restriction sites, such as AvrII and XbaI, are inserted between each potential cis-regulatory element in an alternating fashion. With such example, DNA fragment is incubated with AvrII and XbaI restriction enzymes to cut the restriction sites, fragmenting the DNA string. Using T4 ligase, the DNA string fragments are ligated such that the orientation of each potential cis-regulatory element is random, forming the synthetic promoters.

To prepare the synthetic promoters for screening using the High Content Screening methods described herein, the library of synthetic promoters is cloned, for example, via in-fusion cloning into (1) a screening vector backbone comprising a wild-type ITR, and (2) a screening vector backbone comprising a mutant ITR, which has, e.g., a deleted B region (Takara/Clontech). It is contemplated herein that the synthetic promoters are cloned such that they are proximal to the ITR (e.g., the wild-type ITR or the mutant ITR). Next, a plurality of barcodes is integrated into each screening vector backbone such that each vector comprises a plurality of unique barcodes associated with the cis-regulatory element of the synthetic promoter. The screening vector is than analyzed using standard techniques, e.g., next generation sequencing, to identify (1) the plurality of unique barcodes and (2) the cis-regulatory element associated with the plurality of unique barcodes in each vector.

Finally, a minimal promoter and a marker gene, e.g., a green fluorescent protein (GFP) marker gene, are cloned into the screening vector backbone, e.g., via in-fusion cloning. To maintain a high complexity, it is important to ensure a 5-fold excess with each cloning step.

Next, in order to measure the strength of a liver promoter in vitro, the screening vectors are stably expressed in a hepatocyte using standard techniques, such as lipid-based transfection. It is specifically contemplated herein that a promoter is measured using methods described herein in the environment from which it is derived; e.g., activity of a liver-specific promoter will be assessed in a liver cell. mRNA is extracted from hepatocytes having stable expression of the liver promoter construct, e.g., using the protocol for mRNA extraction provided with an mRNA extraction kit obtained from ThermoFisher (catalog number 61006). mRNA is purified and used as a template to synthesize cDNA, e.g., the protocol for cDNA synthesis provided with using ProtoScript® First Strand cDNA Synthesis Kit obtained from New England Biolabs (catalog number E6300S).

The barcode sequence is, e.g., PCR-amplified from the cDNA using primers that include index primers and P7 and P5 oligos for direct Illumina sequencing. The left primer (leftBC) has a sequence of CAAGCAGAAGACGGCATACGAGATACGAGACTGATTAGTCAGTCAGCCCTCCG CCTTGCCCTGA (SEQ ID NO: 7), and the right primer (Right_UPAS) has a sequence of AATGATACGGCGACCACCGAGATCTACACTATGGTAATTGTTCCTACTATTCCG TACCGTAGGGT (SEQ ID NO: 8). Sequencing is used to measure the content of each of the plurality of barcodes present in a given amplicon. This amplified content of each of the barcode is the barcode output. The barcode output is normalized to the barcode input, which is the content of each unique barcode. The normalized ratio is the expression frequency, and is an indicator of the strength of the URE associated with the barcode in relation to the ITR (e.g., the wild-type ITR or mutant ITR). For example, having a high expression frequency of a barcode in the backbone having a wild-type ITR as compared to the backbone having a mutant ITR indicates that that function of the URE is regulated by the ITR, e.g., the B region of the ITR.

As a proof of concept for this method, five control promoters are spiked into each screening vector library (e.g., with the wild-type ITR or mutant ITR): CMV-IE, CMVmp, EF1a, EGFP, and PGK-EGFP. Each control is associated with 7 distinct barcodes. It is expected that PCR amplification of a barcode within the amplicon can result in artifact into the system. PCR amplification rounds can result in higher copy numbers of a product by nature of the amplification and not necessarily because the barcode was transcribed in the cell. For example, a barcode having a sequence that is more easily amplified may have an augmented copy number after PCR as compared to a barcode sequence with a different sequence. By analyzing a promoter coupled with 7 distinct barcodes, the effect of artifact can be detected. If the copy number is altered due to PCR of the barcode, we would not expect a similar expression with each promoter. However, data presented herein show that the expression frequency for each promoter is consistent with all 7 distinct barcodes, indicating that the expression frequency is not an artifact due to PCR amplification.

Next, in order to measure the strength of a liver promoter in vivo, the screening vectors are cloned into an AAV vector using standard techniques. AAV vectors are produced using standard techniques in the art, e.g., as described herein above. AAV vectors comprising the components described herein are administered to a mouse via hydrodynamic tail vein injection such that that AAV vectors are expressed in the liver. Prior to administration, the AAV genomes are analyzed via sequencing to determine the barcode frequency present in the input DNA that will be the barcode input.

To measure the barcode output, mice are euthanized and livers are retrieved using standard techniques. Livers are homogenized and mRNA is extracted using an mRNA extract kit obtained from ThermoFisher. mRNA is purified and used as a template to synthesize cDNA using ProtoScript® First Strand cDNA Synthesis Kit obtained from New England Biolabs (catalog number E6300S).

Similar to in vitro measuring, the barcode sequence is amplified from the cDNA and sequenced to measure the amount of each plurality of barcodes is present in a given amplicon. The barcode output is normalized to the barcode input, which is the unique barcode content before amplification. The normalized ratio is the expression frequency, and is an indicator of the strength of the cis-regulatory element associated with the barcode. Additionally, as performed in the in vitro measuring, the five promoters associated with 7 distinct barcodes are expressed in the liver and measured as described above. Again, expression frequency for each promoter is consistent with all 7 distinct barcodes, indicating that the expression frequency is not an artifact of the barcode. Thus, further validating our system for measuring the strength of a promoter in vivo

III. Conformational Change

Various aspects of the invention provide methods for determining the how conformation of a vector, e.g., viral vector, and changes to that conformation, effects the function of a regulatory element. Methods described herein relate to modifying a nucleotide sequence surrounding a URE such that the conformation of the viral vector is altered, thus identifying how the conformation contributes to the function of the URE. In one embodiment, the modified sequence comprises at least one modification, e.g., a nucleotide deletion, substitution, or insertion. In one embodiment, modified sequence comprises at least 2, 3, 4, 5, 6, or more modifications. In one embodiment, the modification is proximal to the URE. In an alternate embodiment, the modification is positioned away from the URE, e.g., at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs from the URE.

As used herein, “conformation” refers to the overall three-dimensional (3D) arrangement of a viral vector, e.g., the tertiary structure of the vector. Viral vectors present in various conformations, for example, a viral vector can form, e.g., a circular vector, an episomal structure, a “doggy-dog structure”, a concatemer, etc. Vector confirmations are known in the art and further described in, e.g., Penuad-Budloo, M., et al. Journal of Virology. August 2008, p. 7875-7885; and Nakai, H., et al. Molecular Therapy. 7(1), January 2003, the contents of which are incorporated herein by reference in their entireties. As used herein, a “conformational change” refers to the degree of change in conformation of a viral vector having at least one modification as compared to the conformation of an unmodified (e.g., not having the at least one mutation) viral vector under normal conditions, e.g., native (e.g., the same) conditions. In one embodiment, the conformation of a viral vector is changed by the at least one mutation found within the viral vector, the URE, the DRE, etc. For example, the mutation inhibits the conformation, alters the conformation (such that it undergoes a distinctly different conformational change), or a promotes the conformation more readily as compared to a wild-type, unmodified viral vector under normal conditions. One skilled in the art can determine if a modification alters the conformation of a viral vector, e.g., by using standard techniques in the art, such as X-ray crystallography (e.g., high resolution of the conformation); nuclear magnetic resonance (NMR) (e.g., lower resolution of protein structure; can provide information about conformational changes); Cryogenic electron microscopy (cryo-EM) (e.g., to show both a protein's tertiary and quaternary structure and Dual polarisation interferometry (e.g., provides information regarding structure and conformation changes over time), and sensitive PCR methods. Alternatively, one can just look at the functional changes relative to an exemplar such as the unaltered sequence under the corresponding conditions.

In one embodiment, it is not necessary to confirm a confirmation change has occurred. It is specifically contemplated herein that a mutation that results in a change in activity (e.g., as assessed by expression of a barcode associated with the mutation) would be a result of a change in confirmation. For example, if a mutation is a conserved change that does not result in a conformational change, it is unlikely to result in a change in activity a barcode associated with the mutation.

In one embodiment, at least one ITR (e.g., the left ITR or right ITR, or both the left and right ITR) comprises a modification resulting in a change in 3D conformation as compared to the corresponding wild type AAV ITR structure. A modified ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In one embodiment, the modified ITR has at least one modification within the loop arm, the truncated arm, and/or the spacer.

In one embodiment, a structural element of the ITR can be modified. For example, the ITR is modified to change the height of the stem and/or the number of nucleotides in the loop. In one embodiment, the height of the stem is at least 2, 3, 4, 5, 6, 7, 8, or 9 nucleotides or more or any range therein. In another example, the loop can have at least 3, 4, 5, 6, 7, 8, 9, or 10 nucleotides or more or any range therein. In one embodiment, the modified ITR functionally interacts with Rep.

In another embodiment, the spacing between two elements of an ITR is modified to be increased or decreased. Exemplary elements include the RBE, a hairpin, arm, a loop, etc. In one embodiment, the spacing increased by at least 1, 2, 3, 4, 5, 6, 7, 8, 9, 10, 11, 12, 13, 14, 15, 16, 17, 18, 19, 20, or more nucleotides. In one embodiment, the spacing decreased by at least 1, 2, 3, 4, 5, 6, 7, 8, 9, 10, 11, 12, 13, 14, 15, 16, 17, 18, 19, 20, or more nucleotides.

In some embodiments, the ITR comprises at least one modification within the functional interaction of the ITR with a large Rep protein (e.g., Rep 78 or Rep 68). In certain embodiments, the at least one modification provides selectivity to the interaction of an ITR with a large Rep protein, i.e., determines at least in part which Rep protein functionally interacts with the ITR. In other embodiments, the at least one modification is within a structural element that physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution site (trs). In one embodiment, a modified ITR does not contain any nucleotide deletions in the RBE-containing portion of the A or A′ regions, so as not to interfere with DNA replication (e.g. binding to a RBE by Rep protein, or nicking at a terminal resolution site). In one embodiment, the ITR structure can be modified such that it has a different 3D conformation with respect to the 3D conformation of the wild type ITR structure, but still retains an operable RBE, trs and RBE′ portion.

In one embodiment, the ability of a structural element to functionally interact with a particular large Rep protein can be altered by modifying the structural element of the ITR. In one embodiment, one or more structural element (e.g., A arm, A′ arm, B arm, B′ arm, C arm, C′ arm, D arm, RBE, RBE′, and trs) of an ITR can be modified as defined herein. In one embodiment, one or more structural element can be removed, or replaced with a structural element from a different parvovirus, e.g., a different AAV or non-AAV species. In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present. In one embodiment, a modification in the A, A′, B, B′, C, C′, D or D′ regions, still preserves the terminal loop of the stem-loop. In one embodiment, a modification in the A, A′, B, B′, C, C′, D or D′ regions, still alters the terminal loop of the stem-loop.

In one embodiment, the modified can have at least 80%, at least 85%, at least 90%, at least 95%, at least 96%, at least 97%, at least 98%, at least 99%, or more sequence identity with the corresponding ITR, or wild-type ITR without the modification.

As disclosed herein, a modified ITR can be generated to include a deletion, insertion, or substitution of one or more nucleotides from the wild-type ITR derived from AAV genome. The modified ITR can be generated by genetic modification during propagation in a plasmid in Escherichia coli or as a baculovirus genome in Spodoptera frugiperda cells, or other biological methods, for example in vitro using polymerase chain reaction, or chemical synthesis.

In one embodiment, a viral vector comprises at least one modification that induces a conformational change in the viral vector. In one embodiment, the regulatory element, e.g., a URE, is proximal to the TR (e.g., an ITR), and the modification increases the space between the URE and a TR. In one embodiment, the URE is proximal to the TR, and the modification decreases the distance between the URE and a TR. In one embodiment, the distance between the URE and the TR is increased by at least 1, 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, or more nucleotides. In one embodiment, the distance between the URE and the TR is decreased by at least 1, 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, or more nucleotides. In one embodiment, the URE is proximal to the TR, and the modification alters the TR, e.g., alters the size, structure, function, etc.

In one embodiment, the URE is located within the TR (e.g., an ITR), and the modification increases the size of the TR, e.g., the modification increases the TR by at least 2, 3, 4, 5, 6, 7, 8, 9, 10, 11, 12, 13, 14, 15, 16, 17, 18, 19, 20, 21, 22, 23, 24, 25, 50, 100, or more nucleotides. In one embodiment, the URE is located within the TR, and the modification decreases the size of the TR, e.g., the modification increases the TR by at least 2, 3, 4, 5, 6, 7, 8, 9, 10, 11, 12, 13, 14, 15, 16, 17, 18, 19, 20, 21, 22, 23, 24, 25, 50, 100, or more nucleotides.

In one embodiment, the viral vector is an AAV vector and the URE is proximal to an ITR, and the modification increases the space between the URE and the ITR. In one embodiment, the viral vector is an AAV vector and the URE is proximal to an ITR, and the modification decreases the space between the URE and the ITR. In one embodiment, the distance between the URE and the ITR is increased by at least 1, 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, or more nucleotides. In one embodiment, the distance between the URE and the ITR is decreased by at least 1, 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, or more nucleotides. In one embodiment, the viral vector is an AAV vector and an URE is proximal to the ITR, and the modification is a mutation within the ITR.

In one embodiment, the viral vector is an AAV vector and the URE is located within an ITR, and the modification increases the size of the ITR, e.g., the modification increases the ITR by at least 2, 3, 4, 5, 6, 7, 8, 9, 10, 11, 12, 13, 14, 15, 16, 17, 18, 19, 20, 21, 22, 23, 24, 25, 50, 100, or more nucleotides. In one embodiment, the viral vector is an AAV vector and the URE is located within an ITR, and the modification decreases the size of the ITR, e.g., deletes a loop of the ITR, or decreases the ITR by at least 2, 3, 4, 5, 6, 7, 8, 9, 10, 11, 12, 13, 14, 15, 16, 17, 18, 19, 20, 21, 22, 23, 24, 25, 50, 100, or more nucleotides.

In one embodiment, the parvovirus is a dependovirus and the at least one modification that results in a conformational change is in at least one of the A, A′, B, B′, C, or C′ loops.

In one embodiment, the parvovirus is an adeno-associated virus (AAV) and the at least one modification that results in a conformational change is in at least one of the A, A′, B, B′, C, C′, D, D′ regions.

Lentiviruses, such as HIV, has trans-acting elements, as well as cis-acting elements. For example, with HIV, both TAT and Rev proteins are trans-acting elements.

In one embodiment, the viral vector is a lentiviral vector, the DRE is TAT or associated with TAT, and the at least one modification that results in a conformational change is made in the TAR RNA stem.

In one embodiment, the viral vector is a lentiviral vector, the DRE is TAT or associated with TAT, and the at least one modification that results in a conformational change is made in the UU-rich bulge.

In one embodiment, the viral vector is a lentiviral vector, the DRE is REV or associated with REV, a REV Responsive Element (RRE) is present in the nucleic acid, and the at least one modification that results in a conformational change is made in the RRE.

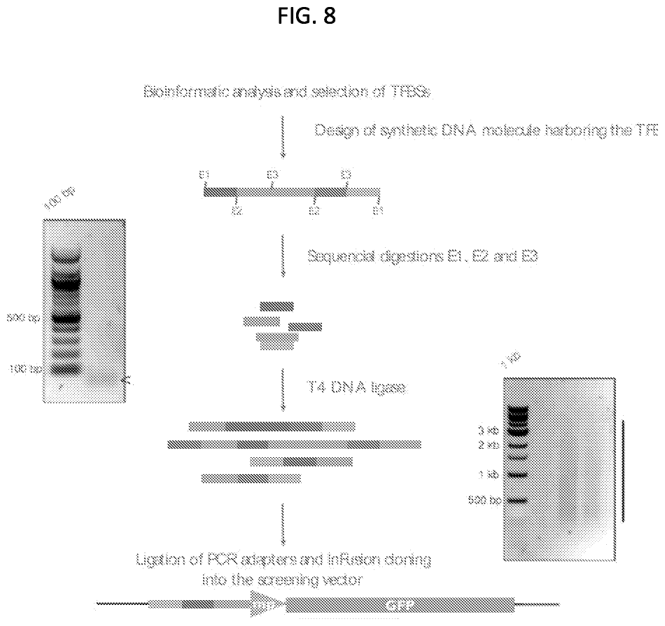

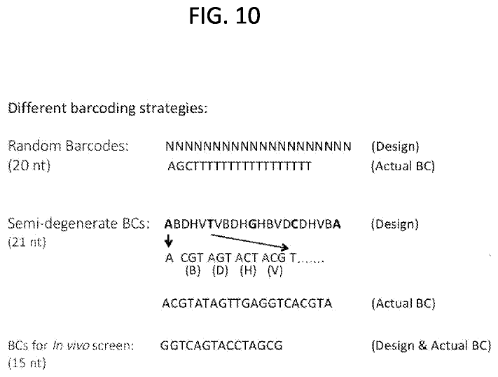

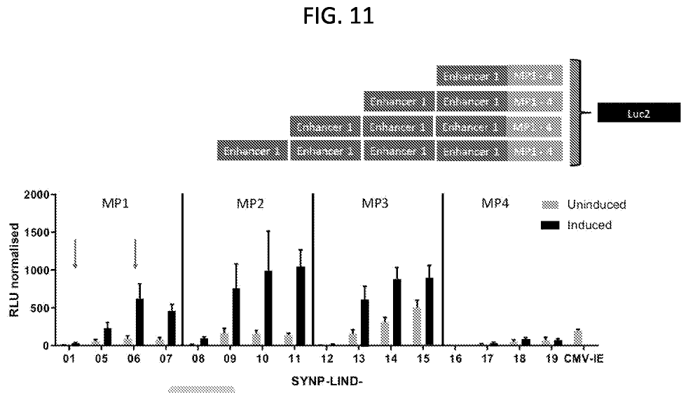

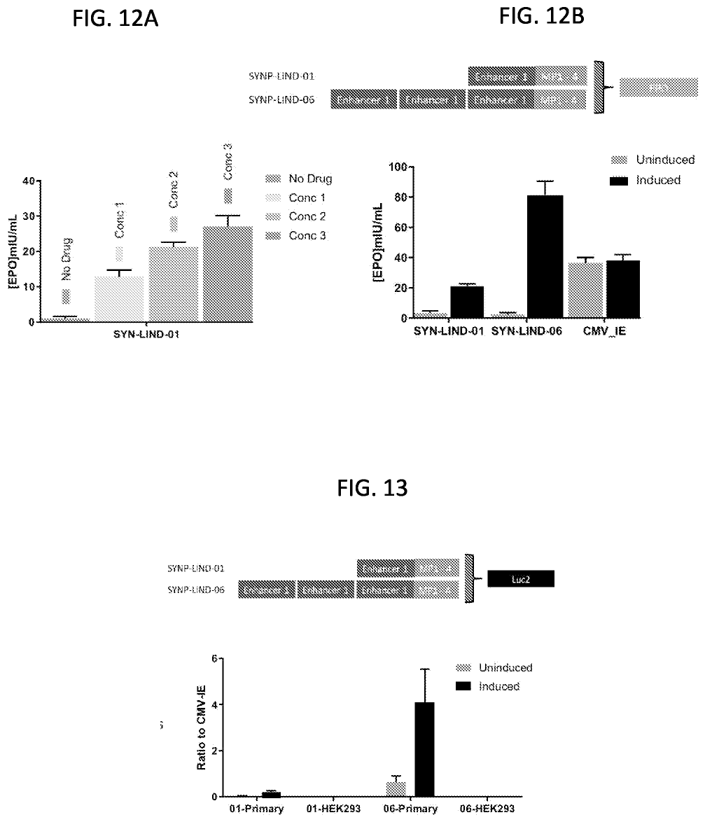

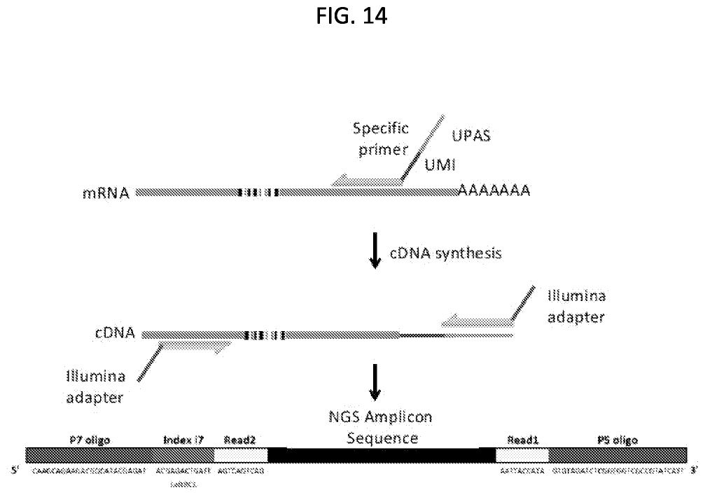

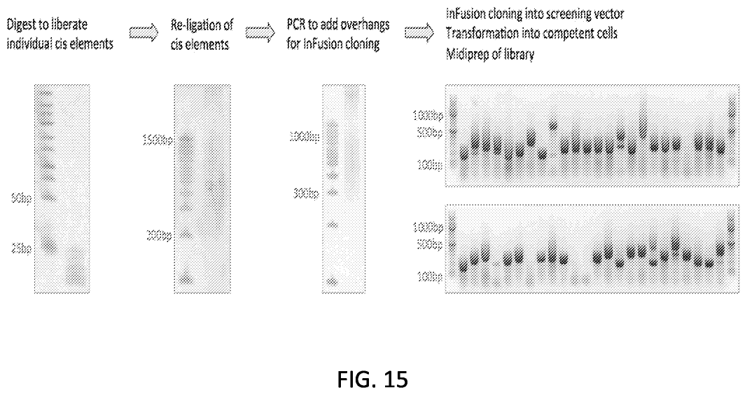

In one embodiment, the viral vector is a dependovirus and the at least one modification that results in a conformation change is in at least one of the A, A′, B, B′, C, or C′ loops. In another embodiment, the viral vector is an AAV virus and the at least one modification that results in a conformation change is in at least one of the A, A′, B, B′, C, or C′ loops. The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See, e.g., FIGS. 8-19; FIELDS et al. VIROLOGY, volume 2, chapter 69 (4th ed., Lippincott-Raven Publishers). A number of relatively new AAV serotypes and clades have been identified (See, e.g., Gao et al. (2004) J. Virol. 78:6381; Moris et al. (2004) Virol. 33-:375). References cited herein are incorporated herein by reference in their entireties.

IV. Transcribable Reporter Sequence

In one embodiment, the plurality of UREs is operatively linked to a transcribable reporter sequence, e.g., an open reading frame (ORF), thus regulating expression of said ORF. A transcribable reporter sequence of the invention can be, for example, any open reading frame that has the ability to be translated to a protein in the host cell. In one embodiment, the transcribable reporter sequence is the ORF of a marker gene. As used herein, “marker gene” refers to a gene whose gene product can be visualized using various methods, but has no biological function. Exemplary marker genes include fluorescent proteins, such as Green Fluorescent Protein, Cherry Fluorescent Protein, or Yellow Fluorescent Protein; a luminescent protein, such as luminescent protein, renilla protein, or nanoluciferase protein; or an epitope tag, such as Myc tag, FLAG tag, V5 tag, or HA tag. One skilled in the art can visualize a marker gene using standard techniques, e.g., fluorescent microscopy to visualize a fluorescent protein; a plate reader to visualize a luminescent protein; or western blotting to detect expression of an epitope tag. Additionally, genome sequencing can be used to measure the quantity of the marker gene in the cell. It is desired that the open reading frame does not have biological function that will interfere with the biological properties of the cell it is expressed in.

In an alternate embodiment, the transcribable reporter sequence is the ORF of any gene having a biological function such as a therapeutic function. It is understood that the transcribable reporter sequence can be the ORF of any known, or yet to be discovered, gene, without limitation to its function, cellular localization, expression pattern, etc. The transcribable reporter sequence can be the ORF of any known disease gene, i.e., a gene bearing a mutation, as compared to the wild-type gene, that results in a disease or disorder.

As disclosed herein, the present invention also provides an expression construct or vector comprising a URE as set out above, operably linked to an ORF, wherein the ORF comprises a nucleic acid sequence encoding an expression product. The expression construct or vector can be any expression construct or vector as discussed above for the other aspects of the invention. The expression product encoded by the ORF can be any expression product (e.g. encoding a protein). In some embodiments the expression product is not a reporter protein, i.e. it does not encode a protein that is used conventionally as an indicator of expression levels. Many reporter genes are known in the art, including, in particular, fluorescent, luminescent proteins and chromogenic proteins. Thus, in some embodiments, the expression product is not a fluorescent or luminescent protein, e.g. it is not a luciferase.

In some embodiments, an expression product encoded by the ORF is a therapeutic protein (e.g., therapeutic polypeptides) or toxic protein. Therapeutic polypeptides include, but are not limited to, cystic fibrosis transmembrane regulator protein (CFTR), dystrophin (including mini- and micro-dystrophins, see, e.g., Vincent et al., (1993) Nature Genetics 5:130; U.S. Patent Publication No. 2003/017131; International Patent Publication No. WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factor-α soluble receptor), S100A1, parvalbumin, adenylyl cyclase type 6, a molecule that modulates calcium handling (e.g., SERCA2A, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)).

In some embodiments, the expression product encoded by a ORF is a reporter polypeptide (e.g., an enzyme). Reporter polypeptides are known in the art and include, but are not limited to, Green Fluorescent Protein (GFP), luciferase, 0-galactosidase, alkaline phosphatase, and chloramphenicol acetyltransferase gene.

In alternative embodiments, the expression product encoded by the ORF is a secreted polypeptide (e.g., a polypeptide that is a secreted polypeptide in its native state or that has been engineered to be secreted, for example, by operable association with a secretory signal sequence as is known in the art).

IV. Barcodes

The invention provides for the inclusion of a plurality of nucleic acid barcodes unique to a specific URE to facilitate the determination of the strength of said URE with precision and accuracy. The pluralities of barcodes are associated with at least one URE, comprising a combination of regulatory elements, such that they are transcribed in the same mRNA transcript as the associated open reading frame. Barcodes may be oriented in the mRNA transcript 5′ to the open reading frame, 3′ to the open reading frame, immediately 5′ to the terminal poly-A tail, or somewhere in-between. Following construction of a plurality of synthetic nucleic acids or libraries thereof, the synthetic nucleic acid is sequenced to identify (1) the URE comprised within the synthetic nucleic acid, and (2) the associated unique barcode. This information can be categorized to construct a database showing the unique barcode that corresponds with a given URE. While barcodes have been proposed in a number of systems, we have discovered that the barcodes selected can sometimes affect complexity of the library effect results. For example, amplicon generation by PCR may introduce stochasticity bias (non-uniform amplification). The homopolymer run in a barcode should not be greater than 5 bp. In one embodiment, it should not be greater than 4 bp. In another embodiment, it should not be greater than 3 bp. In still another embodiment, it should not be greater than 2 bp. A barcode cannot end with a homopolymer.

In one embodiment, 4-mers cannot be repeated within the barcode. For example, the sequence “ATTC” cannot be present twice within one barcode.

In one embodiment, the barcode should contain all 4 bases. In one embodiment, the content of A and T must be at least 20%. In one embodiment, the content of G and C must be at least 12.5%.

A plurality of unique barcodes contains at least 1, 2, 3, 4, 5, 6, 7, 8, 9, 10, 11, 12, 13, 14, 15, 16, 17, 18, 19, 20, 21, 22, 23, 24, 25 or more barcodes. In one embodiment, a synthetic nucleic acid contains only a single unique barcode. In one embodiment, the plurality of barcodes is preferably less than 12 and in more preferred embodiment, it is less than 10.

A barcode described herein is between 12-35 nucleotides in length and has a GC content between 25-65%. The GC content refers to the proportion of G and C bases out of the four bases (i.e., G, C, A, and T/U) in the barcode. GC-content is usually expressed as a percentage value and can be calculated using the following equation: (G+C)/(A+T/U+G+C)×100, wherein each letter in the equation represents the number of corresponding bases present in the sequence of interest. GC content of a primer is often correlated with the annealing temperature, e.g., higher GC content often indicates a high annealing temperature. GC content of a primer is also associated with the stability of the primer, e.g., a primer having a GC content of 40-60% ensure more stable binding of the primer and template. Higher annealing temperatures due to increased GC content lowers the stability of binding the primer and template.

In one embodiment, a barcode is between 12-25 nucleotides in length. In another embodiment, a barcode is between 12-28 nucleotides in length. In yet another embodiment, a barcode is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. In one embodiment, a barcode for use in vitro is about 18-32 nucleotides, 20-28 nucleotides, 21, 22, 23, 24, 25, 26, 27, or 28 nucleotides, e.g., 21 nucleotides in length. In another embodiment, a barcode for use in vivo is 12-18 nucleotides, 12, 13, 14, 15, 16, 17, or 18 nucleotides, e.g., 15 nucleotides in length.

The barcodes described herein can be quantified by methods known in the art, including quantitative sequencing or quantitative hybridization techniques (e.g., microarray hybridization technology). Barcodes described herein can be further be modified for analysis via next generation sequencing (e.g., using an Illumina® sequencer). In one embodiment, the synthetic nucleic acid containing the barcode further comprises at least one unique molecular identifier (UMI). In another embodiment, the above said synthetic nucleic acid contains at least 1, 2, 3, 4, 5, 6, 7, 8, 9, 10, 11, 12, 13, 14, 15, 16, 17, 18, 19, 20, 21, 22, 23, 24, 25 UMI tags. In one embodiment, the synthetic nucleic acid further comprises at least one unique primer annealing sites (UPAS) tag. As used herein, “UPAS” refers to two synthetically generated sequences which do not exist in the mouse genome and have been integrated as primer binding sites for amplicon generation PCR. In another embodiment, said synthetic nucleic acid contains at least 1, 2, 3, 4, 5, 6, 7, 8, 9, 10, 11, 12, 13, 14, 15, 16, 17, 18, 19, 20, 21, 22, 23, 24, 25 UPAS tags. As used herein, “UMI” refers to molecular tags that detect and quantify unique mRNA transcripts. mRNA libraries are generated when plasmids, expression vectors or viral vectors comprising the library (or the plurality of synthetic nucleic acid, as disclosed herein) are expressed in vitro or in vivo. In the reverse transcription process of the mRNA i.e., during the cDNA synthesis, primers used contained UMI sequence, thereby integrating the UMI in the synthesized cDNA. Incorporation of UMI allows additional tagging of each cDNA providing a control for PCR amplification. Sequencing allows for high-resolution reads, enabling accurate detection of unique barcodes coupled with specific URE. Use of UMI tags eliminate PCR-based amplification error (e.g., artifact copies produce via PCR amplification) in the output. Methods utilizing UMI and UPAS tags are further described in, e.g., Kivioja T., et al. (2012) Counting absolute numbers of molecules using unique molecular identifiers. Nat Methods 9: 72-74, the contents of which are incorporated herein by reference in its entirety.

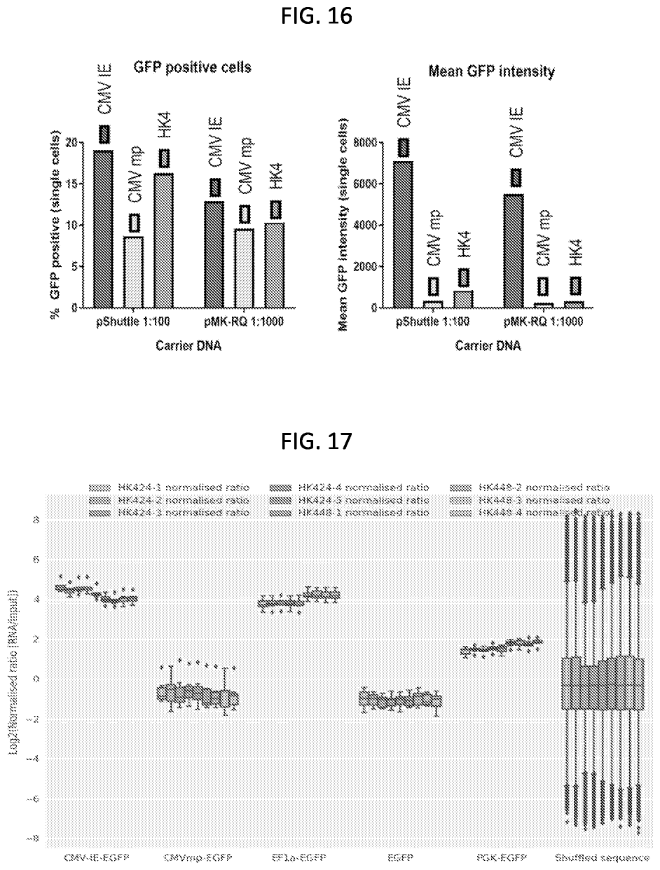

In one embodiment, the barcode sequence is amplified from the cDNA using primers that include index primers and P7 and P5 oligos for direct Illumina sequencing. Sequencing is used to measure the content of each of the plurality of barcodes present in a given amplicon, e.g., that comprises a UMI and/or UPAS. This amplified content of each of the barcode is the barcode output. The barcode output is normalized to the barcode input, which is the content of each unique barcode. The normalized ratio is the expression frequency, and is an indicator of the strength of the URE associated with the barcode. For example, having a high expression frequency of a barcode indicates that the URE or in particular, the unique combination of associated cis-regulatory elements is robust. See, e.g., FIG. 16.

The nucleic acid sequence of unique barcodes described herein have been optimized for the highest efficiency in analysis, e.g., via sequencing. In one embodiment, the nucleic acid sequence of barcodes described herein comprise at least one of each adenine, thymine, guanine, and cytosine. In one embodiment, the nucleic acid sequence of the barcode does not contain tracts of more than three homopolymers in succession. In one embodiment, the nucleic acid sequence of the barcode does not contain tracts of more than two homopolymers in succession. As used herein, “homopolymer” refers to regions of DNA sequence that include stretches of the same nucleotide (e.g. AAAAA or TTTTTTTT). Alternatively, homopolymer containing pairs of the same nucleotides, e.g., dimers (e.g., AATTCC), would be excluded from the barcode. Said another way, a dimer cannot be directly repeated. However, dimers can be repeated within the barcode sequence up to 3 times, e.g., with at least one bp separating each dimer. Long homopolymers are undesirable as it has been found that nucleotides surrounded by long strings of similar nucleotides are often mis-read when analyzed via sequencing. In one embodiment, the nucleic acid sequence of a unique barcode comprising semi-degenerate bases. As used herein, “semi-degenerate bases” refers to a nucleotide that can perform the same function or yield the same output as a structurally different nucleotide. A position of a codon is said to be a fourfold degenerate site if any nucleotide at this position specifies the same amino acid. For example, the third position of the glycine codons (GGA, GGG, GGC, GGU) is a fourfold degenerate site, because all nucleotide substitutions at this site are synonymous; i.e., they do not change the amino acid. There is only one threefold degenerate site where changing to three of the four nucleotides may have no effect on the amino acid (depending on what it is changed to), while changing to the fourth possible nucleotide always results in an amino acid substitution. This is the third position of an isoleucine codon: AUU, AUC, or AUA all encode isoleucine, but AUG encodes methionine. A position of a codon is said to be a twofold degenerate site if only two of four possible nucleotides at this position specify the same amino acid. For example, the third position of the glutamic acid codons (GAA, GAG) is a twofold degenerate site. In twofold degenerate sites, the equivalent nucleotides are always either two purines (A/G) or two pyrimidines (C/U), so only transversional substitutions (purine to pyrimidine or pyrimidine to purine) in twofold degenerate sites are nonsynonymous. A position of a codon is said to be a non-degenerate site if any mutation at this position results in amino acid substitution.

In one embodiment, the nucleic acid sequence of a barcode does not contain the nucleic acid sequence of a restriction enzyme recognition site. Restriction enzyme recognition sites are well known in the art; a skilled person can determine if a barcode nucleic acid sequence contains a recognition site via, e.g., analyzing the sequence via NCBI Basic Local Alignment Search Tool (BLAST).

In one embodiment, the barcode has a hamming distance greater than 2 when compared to other barcodes within the plurality of barcodes. As used herein, “hamming distance” refers to the number of positions at which the corresponding symbols, e.g., nucleotides are different. Said another way, “hamming distance” measures the minimum number of substitutions required to change one nucleotide string into the other, or the minimum number of errors that could have transformed one nucleotide string into the other. Hamming distance can only be measured between sequences having the same length. One skilled in the art can assess the hamming distance of a unique barcode within a library described herein, e.g., using the function d=min {d(x,y):x,y∈C,x≠y}. Alternatively, the distance can be measured using other methods known in the art, e.g., the Damerau-Levenshtein distance.

In one embodiment, a unique barcode has a complexity of at least 4.3×10⁷, at least 2.7×10⁸, or at least 1×10¹². In an alternate embodiment, the unique barcode has a complexity of at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, or more. As used herein, “complexity” refers to the number of possible unique instances in the unique barcodes.

It is desired that a unique barcode for in vivo use has (1) no greater than three homopolymers in succession, (2) a GC content between 25-65%, (3) contain at least one of each nucleic acids (i.e., adenine, thymine, guanine, and cytosine), (4) does not comprising the nucleic acid sequence of a restriction site, (5) has a hamming distance greater than two, and (6) has a complexity of 2.7×10⁸.

IV. Terminal Repeats

In one aspect, the at least one DRE is present within a terminal repeat (TR), or a portion thereof. In various embodiments, the at least one URE is located within 200-500 base pairs of the at least one TR, or portion thereof, or within 20-200 base pairs of the at least one TR, or portion thereof. In an alternative embodiment, the at least one URE is located at least 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs of the at least one TR, or portion thereof.

In one embodiment, the “portion thereof” of a TR refers to a sequence of any length derived from a full length TR sequence. In one embodiment, the “portion thereof” of a TR comprises the function of a full length TR. In one embodiment, “portion thereof” of a TR does not comprise the function of a full length TR, or does not comprise 100% of the function of a full length TR, e.g., functions as a reduced rate. The “portion thereof” of a TR can be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more of the sequence of the full length TR.

In one embodiment, the DRE or URE are proximal to or within a Holliday junction and a change in at least one of the Holliday junctions is made. Holliday junctions are branched nucleic acid structure that contains four double-stranded arms joined together which function as intermediates during DNA recombination and double-stranded break repair. Holliday junctions are typically is a T-shaped or Y-shaped hairpin structure, where each ITR is formed by two palindromic arms or loops (B-B′ and C-C′) embedded in a larger palindromic arm (A-A′), and a single stranded D sequence, as described in, e.g., U.S. Pat. No. 5,4478,784 (Samulski et al.), which is incorporated herein by reference, where the order of these palindromic sequences defines the flip or flop orientation of the ITR (e.g., the left or right ITR). Holliday junctions can be mobile, meaning the junction has symmetrical sequences that allow for “sliding.” Holliday junctions can additionally be immobile, meaning they have asymmetrical sequences that are “locked.” A change in the Holliday junction proximal to the DRE or URE, for example a nucleotide substitution, deletion, or addition is made can alter, e.g., the state (e.g., from mobile to immobile), the function, the structure (e.g., 2 vs. 4 strands), or any aspect of the Holliday junction. In one embodiment, a nucleic acid sequence described herein comprises a change, e.g., a nucleotide substitution, deletion, or addition, that results in the formation of a Holliday junction. A Holliday junction can be naturally occurring or result from at least one addition, substitution, or deletion of a nucleic acid. In one embodiment, the Holliday junction is a wild-type Holliday junction. In one embodiment, the Holliday junction is a mutant or synthetic Holliday junction. For example, the Holliday junction which the DRE is proximal to can be changed, or another Holliday junction can be changed. Alternatively, more than one Holliday junction, e.g., the Holliday junction which the DRE is proximal to and at least one additional Holliday junction, can be changed. In one embodiment, the Holliday junction is formed from at least one modification, e.g., at least one addition, substitution, or deletion of a nucleic acid. For example, a sequence can be modified to induce the formation of a Holliday junction in a sequence that does not comprise a naturally occurring Holliday sequence. Holliday junctions are known in the art and can be readily identified using standard techniques for identifying RNA structure, e.g., crystallography approaches.

In one embodiment, the synthetic nucleic acid described herein comprises at least one TR or portion thereof.

In various embodiments, the TR is an ITR, or a portion thereof, e.g., a sequence of any length derived from a full length ITR sequence. In one embodiment, the “portion thereof” of an ITR comprises the function of a full length ITR. In one embodiment, “portion thereof” of an ITR does not comprise the function of a full length ITR, or does not comprise 100% of the function of a full length ITR, e.g., functions as a reduced rate. The “portion thereof” of a ITR can be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more of the sequence of the full length ITR.

An ITR includes any viral TR or synthetic sequence that forms a hairpin structure and functions as an ITR (i.e., mediates the desired functions such as replication, integration and/or provirus rescue, and the like).

An AAV ITR may be from any parvovirus, for example a dependovirus such as AAV, including but not limited to serotypes AAV1, AAV2, AAV 3a, AAV3b, AAV4, AAV5, AAV6, AAV7, AAV8, AAV9, AAV10, AAV11, AAV12, or AAV13 ITR, snake AAV, avian AAV, bovine AAV, canine AAV, equine AAV, ovine AAV, goat AAV, shrimp AAV, or any other AAV now known or later discovered. An AAV ITR need not have the native terminal repeat sequence (e.g., a native AAV ITR sequence may be altered by insertion, deletion, truncation and/or missense mutations), as long as the terminal repeat mediates the desired functions, e.g., replication, or, integration, e.g., NCBI: NC 002077; NC 001401; NC001729; NC001829; NC006152; NC 006260; NC 006261), chimeric ITRs, or viruses of the Parvoviridae family, e.g., Parvovirinae or Densovirinae. In some embodiments, the AAV can infect warm-blooded animals, e.g., avian (AAAV), bovine (BAAV), canine, equine, and ovine adeno-associated viruses. In some embodiments the ITR is from B19 parvoviris (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). An AAV ITR need not have the native TR sequence (e.g., a native AAV ITR sequence may be altered by insertion, deletion, truncation and/or missense mutations), as long as the TR mediates the desired functions, e.g., replication, or, integration.

The ITR can be a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, e.g., as described in U.S. Pat. No. 9,169,494, the contents of which are incorporated by reference in their entirety. Typically, the ITR is 145 nucleotides. The terminal 125 nucleotides form a palindromic double stranded T-shaped hairpin structure. In the structure the A-A′ palindrome forms the stem, and the two smaller palindromes B-B′ and C-C′ form the cross-arms of the T. The other 20 nucleotides in the D sequence remain single-stranded. In the context of an AAV genome, there would be two ITR's, one at each end of the genome.

In one embodiment, the ITR is a wild-type ITR. In another embodiment, the ITR is a mutant ITR. A mutant ITR can be a functional or non-functional ITR. For example, a non-functional ITR would have reduced or a complete loss of the function of a wild-type ITR, e.g., mediates replication, integration and/or provirus rescue.

In one embodiment, the TR, or portion thereof, comprises at least one modification. A modification can be, e.g., base pair addition, deletion, or substitution. In one embodiment, the at least one TR, e.g., an ITR, comprises at least 1, 2, 3, 4, 5, 6, or more modifications. In one embodiment, the at least 1, 2, 3, 4, 5, 6, or more modifications in a given TR, or portion thereof, are associated with the same plurality of barcodes. In an alternative embodiment, the at least 1, 2, 3, 4, 5, 6, or more modifications in a given TR, or portion thereof, are associated with at least two different pluralities of barcodes.

One can modify an ITR sequence from any AAV serotype for use herein, for example, AAV serotype 1 (AAV1), AAV serotype 2 (AAV2), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12). The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. In one embodiment, ITRs from a combination of different AAV serotypes can be used, e.g., one ITR can be from one AAV serotype and the other ITR can be from a different serotype.

In one embodiment, the mutant ITR is a DD mutant ITR (DD-ITR). A DD-ITR has the same sequence the ITR from which it is derived, but includes a second D sequence adjacent the A sequence, so there are D and D′. The D and D′ can anneal (e.g., as described in U.S. Pat. No. 5,478,745, the contents of which are incorporated herein by reference). Each D is typically about 20 nucleotides (nt) in length, but can be as small as 5 nucleotides. Shorter D regions preserve the A-D junction (e.g., are generated by deletions at the 3′ end that preserve the A-D junction). Preferably the D region retains the nicking site and/or the A-D junction. The DD-ITR is typically about 165 nucleotides. The DD-ITR has the ability to provide information in cis for replication of the DNA construct. Thus, a DD-ITR has an inverted palindromic sequence with flanking D and D′ elements, e.g. a (+) strand 5′ to 3′ sequence of 5′-DABB′CC′A′D′-3′ and a (−) strand complimentary to the (+) strand that has a 5′ to 3′ sequence of 5′-DACC′BB′A′D′-3′ that can form a Holiday structure, e.g. as illustrated in FIG. 1. In certain embodiments, the DD-ITR may have deletions in its components (e.g. A-C), while still retaining the D and D′ element. In certain embodiments, the ITR comprises deletions while still retaining the ability to form a Holliday structure and retaining two copies of the D element (D and D′). The DD-ITR may be generated from a native AAV ITR or from a synthetic ITR. In certain embodiments, the deletion is in the B region element. In certain embodiments, the deletion is in the C region element. In certain embodiments, a deletion within both the B and C element of the ITR. In one embodiment, the entire B and/or C element is deleted, and e.g., replaced with a single hairpin element. In one embodiment, the template comprises at least two DD-ITRs.

A synthetic ITR can also be used. The synthetic ITR refers to a non-naturally occurring ITR that differs in nucleotide sequence from wild-type ITRs, e.g., the AAV serotype 2 ITR (ITR2) sequence due to one or more deletions, additions, substitutions, or any combination thereof. The difference between the synthetic and wild-type ITR (e.g., ITR2) sequences may be as little as a single nucleotide change, e.g., a change in 1, 2, 3, 4, 5, 6, 7, 8, 9, 10, 15, 20, 25, 30, 35, 40, 45, 50, 60, 60, 70, 80, 90, or 100 or more nucleotides or any range therein. In some embodiments, the difference between, the synthetic and wild-type ITR (e.g., ITR2) sequences may be no more than about 100, 90, 80, 70, 60, 50, 45, 40, 35, 30, 25, 20, 15, 10, 9, 8, 7, 6, 5, 4, 3, 2, or 1 nucleotide or any range therein.

ITRs can form an intramolecular duplex secondary structure, e.g., modified ITRs where part of the stem-loop structure is deleted, or ITRs comprising a single stem and two loops, or a single stem and single loop. Secondary structures of ITRs are inferred or predicted based on the ITR sequences. Secondary structures can be inferred, e.g., using thermodynamic methods based on nearest neighbor rules that predict the stability of a structure as quantified by folding free energy change or by finding the lowest free energy structure; an algorithm disclosed in Reuter, J. S., & Mathews, D. H. (2010) RNAstructure: software for RNA secondary structure prediction and analysis. BMC Bioinformatics. 11,129 and implemented in the RNAstructure software (available at world wide web address: “rna.urmc.rochester.edu/RNAstructureWeb/index.html”); or RNA structure software that can predict modified T-shaped stem-loop structures with estimated Gibbs free energy (AG) of unfolding under physiological conditions.

Additional TRs can be used in the current invention, for example a long terminal repeat (LTR). In various embodiments, the TR is an LTR, or a portion thereof, e.g., a sequence of any length derived from a full length LTR sequence. In one embodiment, the “portion thereof” of an LTR comprises the function of a full length LTR. In one embodiment, “portion thereof” of an LTR does not comprise the function of a full length LTR, or does not comprise 100% of the function of a full length LTR, e.g., functions as a reduced rate. The “portion thereof” of a LTR can be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more of the sequence of the full length LTR.

V. Viral Vectors

Various aspects of the invention relate to a population of viral vectors or AAV vectors expressing the plurality of synthetic nucleic acids, the library of plasmids, or the library of expression vectors described herein. Methods described herein utilize these viral vectors to identify the strength of a URE in vivo and in vitro.

Synthetic nucleic acids described herein can be used in the production of recombinant vectors, e.g., a recombinant AAV vector. Protocols for producing recombinant vectors and for using vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997). Further, production of AAV vectors is further described, e.g., in U.S. Pat. No. 9,441,206, the contents of which is incorporated herein by reference in its entirety. Nonlimiting examples of vectors employed in the methods of this invention include any nucleotide construct used to deliver nucleic acid into cells, e.g., a plasmid, an expression vector, a template, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus vector can then be administered in vivo and thereby deliver a synthetic nucleic acid of the invention in vivo. The exact method of introducing the synthetic nucleic acids into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). Incubation conditions (e.g., timing, climate, medium, etc.) for a given condition are known in the art and can be readily identified by a skilled practitioner.

Viral vectors produced in a cell can be released (i.e. set free from the cell that produced the vector) using any standard technique. For example, viral vectors can be released via mechanical methods, for example microfluidization, centrifugation, or sonication, or chemical methods, for example using lysis buffers and detergents. Released viral vectors are then recovered (i.e., collected) and purified to obtain a pure population using standard methods in the art. For example, viral vectors can be recovered from a buffer they were released into via purification methods, including a clarification step using depth filtration or Tangential Flow Filtration (TFF). Viral vectors can be released from the cell via sonication and recovered via purification of clarified lysate using column chromatography.

In one embodiment, the viral vector is a DNA or RNA virus. In one embodiment, the viral vector is a parvovirus, a lentivirus, or an adenovirus, an adeno-associated virus (AAV) vector, a retrovirus vector, a herpesvirus vector, an alphavirus vector, a poxvirus vector, a baculovirus vector, and a chimeric virus vector.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Peaenation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Viral vectors of the invention may comprise the genome, in part or entirety, of any naturally occurring and/or recombinant viral vector nucleotide sequence (e.g., AAV, AV, LV, etc.) or variant. Viral vector variants may have genomic sequences of significant homology at the nucleic acid and amino acid levels, produce viral vector which are generally physical and functional equivalents, replicate by similar mechanisms, and assemble by similar mechanisms.

Variant viral vector sequences can be used to deliver a synthetic nucleic acid in vivo as described herein. For example, one or more sequences having at least about 70%, at least about 75%, at least about 80%, at least about 85%, at least about 90%, at least about 95%, at least about 99%, or more nucleotide and/or amino acid sequence identity (e.g., a sequence having about 75-99% nucleotide sequence identity) to a given vector (for example, AAV, AV, LV, etc.). In one embodiment, viral vectors, e.g., AAV vectors are used to express synthetic nucleic acids described herein in vivo. In an alternative embodiment, viral vectors, e.g., AAV vectors are used to express synthetic nucleic acids described herein in vitro.

In one embodiment, the viral vector is an AAV vector. AAV vectors can be an AAV vector from any serotype, e.g., serotypes 1, 2, 3a, 3b, 4, 5, 6, 7, 8, 9, 10, 11, or 13, or species, e.g., snake AAV, avian AAV, bovine AAV, canine AAV, equine AAV, ovine AAV, goat AAV, shrimp AAV, or any other AAV now known or later discovered.

In one embodiment, the viral vector is a wild-type vector, e.g., a wild-type AAV vector. In one embodiment, the viral vector is a mutant vector, e.g., having a sequence that is altered as compared to wild-type, such as a mutant AAV vector, e.g., a DD mutant. In one embodiment, a viral vector comprises at last one modification, e.g., a nucleotide substitution, deletion, or addition. In one embodiment, a viral vector comprises at least 2, 3, 4, 5, 6, 7, 8, 9, 10, 11, 12, 13, 14, 15, 16, 17, 18, 19, 20, or more modifications. A modification can alter the function of the viral vector, e.g., reduce virulence, reduce immunogenicity, increase tropism, alter the rate of replication, or the like. Alternatively, a modification does not have an effect or alter the function of the viral vector. Preferably, in one embodiment, modification can alter the conformation of the viral vector.

In one embodiment, the viral vector is a partial viral vector. In one embodiment, a partial viral vector comprises a TR, a response element, a cis-acting viral element, and a trans-acting viral element.

In one embodiment, the viral vector is an AAV vector and the at least a part of a TR is selected from the group consisting of: an inverted terminal repeat (ITR), an A region, an A′ region, a B region, a B′ region, a C region, a C′ region, a D region, a D′ region, a TRS (terminal resolution site), and a Rep binding site (RBS). In one embodiment, the A region, A′ region, B region, B′ region, C region, C′ region, D region, or D′ region is derived from a wild-type inverted terminal repeat (ITR), a mutant ITR, a truncated ITR, or a synthetic ITR.

In one embodiment, if a synthetic nucleic acid comprised both a DRE and a TR of the viral vector sequence, or partial vector, then the DRE and the TR comprised in the viral vector or the partial vector, are separated by 2-500 base pairs. In one embodiment, if a synthetic nucleic acid comprised both a DRE and a viral vector sequence, or portion thereof, then the DRE and the viral vector, or portion thereof, are separated by 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, 500, 550, 600, 650, 700, 750, 800, 850, 900, 950, 1000, 1500, 10000, 15000 or more base pairs.

It is understood that a viral vector would further comprise components necessary for a given vector. For example, production of an AAV requires the presence of at least one Replication (Rep) genes and/or at least Capsid (Cap) genes. On the left side of the AAV genome there are two promoters called p5 and p19, from which two overlapping messenger ribonucleic acids (mRNAs) of different length can be produced. Each of these contains an intron which can be either spliced out or not, resulting in four potential Rep genes; Rep78, Rep68, Rep52 and Rep40. Rep genes (specifically Rep 78 and Rep 68) bind the hairpin formed by the ITR in the self-priming act and cleave at the designated terminal resolution site, within the hairpin. They are necessary for the AAVS1-specific integration of the AAV genome. All four Rep proteins were shown to bind ATP and to possess helicase activity. The right side of a positive-sensed AAV genome encodes overlapping sequences of three capsid proteins, VP1, VP2 and VP3, which start from one promoter, designated p40. The cap gene produces an additional, non-structural protein called the Assembly-Activating Protein (AAP). This protein is produced from ORF2 and is essential for the capsid-assembly process. Necessary elements for manufacturing AAV vectors are known in the art, and can further be reviewed, e.g., in U.S. Pat. Nos. 5,478,745A; 5,622,856A; 5,658,776A; 6,440,742B1; 6,632,670B1; 6,156,303A; 8,007,780B2; 6,521,225B1; 7,629,322B2; 6,943,019B2; 5,872,005A; and U.S. Patent Application Numbers US 2017/0130245; US20050266567A1; US20050287122A1; the contents of each are incorporated herein by reference in their entireties. In various embodiments, nucleic acids expressing Rep and/or Cap genes are transformed using standard methods, for example, by a plasmid, a virus, a liposome, a microcapsule, a non-viral vector, or as naked DNA.

In one embodiment, expression of a vector, e.g., the AAV vector, is localized to a specific organ or tissue. Exemplary organs or tissues include, the liver (or specifically the liver right lobe, liver left lobe, liver median lobe, liver caudate lobe), spleen, brain, Skeletal Muscle, Heart, Aorta, lungs, blood vessels, pancreas, bladder, reproductive system, small intestine, large intestine, esophagus, rectum, thyroid, diaphragm, stomach, kidney, or the like. In one embodiment, expression of the vector is localized to at least two organs or tissue types. Methods for detecting expression of a vector are known in the art and include, e.g., microscopy of an isolated organ or tissue, or FACS of cells obtained from an isolated organ or tissue. The mode of administration of the vector can be selected to achieve specific expression of the vector in a given tissue or organ. For example, intra-venous administration is used to achieve expression in the muscle, spleen, aorta, liver, lung, heart, and heart; intra-cerebral administration is used to achieve expression in the brain; and intra-muscular administration is used to achieve expression in the muscle.

VI. Libraries of Plasmids and Expression Vectors

One aspect of the invention is a library comprising a plurality of expression vectors or plasmids that express the plurality of synthetic nucleic acids described herein. In one embodiment, the library of expression vectors or plasmids comprises at least 50 expression vectors or at least 50 plasmids that express the plurality of synthetic nucleic acids described herein. In one embodiment, the library of expression vectors or plasmids comprises at least 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10000, 15000, 20000, 25000, 30000, 35000, 40000, 45000, 50000, 55000, 60000, 65000, 70000, 75000, 80000, 85000, 90000, 95000, 100000, or more expression vectors or plasmids that express the plurality of synthetic nucleic acids.

As used herein, a “plasmid” refers to a small, circular piece of DNA, that is distinct from chromosomal DNA and replicated independently of chromosomal DNA. As used herein, “expression vector” refers to a vector that directs expression of a synthetic nucleic acid described herein. One skilled in the art would be able to readily identify a plasmid or expression vector useful for expression a synthetic nucleic acid described herein.

Cloning methods for expressing synthetic nucleic acids in a given expression vector or plasmid are well known in the art, and can be executed by a skilled person. For example, molecular subcloning techniques can be used to introduce a synthetic nucleic acid into an expression vector or plasmid.

The expression vector or plasmid of this invention preferably does not include any additional regulatory element sequence other than those present in the synthetic nucleic acid in which it expresses. This ensures that all gene transcription is being regulated by the URE introduced into the plasmid or expression vector via synthetic nucleic acid expression.

Vectors (e.g., expression vectors and viral vectors) or plasmids may also include additional elements (e.g., invariant promoter elements (e.g., a minimal mammalian TATA box promoter or a synthetic inducible promoter), invariant or low complexity regions suitable for priming first strand cDNA synthesis (e.g., located 3′ of the nucleic acid tag), elements to aid in isolation of transcribed RNA, elements that increase or decrease mRNA transcription efficiency (e.g., chimeric introns) stability (e.g., stop codons), regions encoding a poly-adenylation signal (or other transcriptional terminator), and regions that facilitate stable integration into the cellular genome (e.g., drug resistance genes or sequences derived from lentivirus or transposons).

In one embodiment, the expression vector or plasmid further comprises an antibiotic resistance gene, e.g., a gene that confers resistance to neomycin, zeocin, hygromycin, puromycin, or the like. The expression vector may be any vector capable of expression of an antibiotic resistance gene in the cell or tissue of interest. For example, the vector may be a plasmid or a viral vector. The vector may be a vector that integrates into the host genome, or a vector that allows gene expression while not integrated.

The expression vector can be an integrating vector or a non-integrating vector.

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Yet another example of a non-integrative vector is a minicircle vector. Minicircle vectors are circularized vectors in which the plasmid backbone has been released leaving only the eukaryotic promoter and cDNA(s) that are to be expressed. Further, doggy-bone vectors are another example of non-integrative vectors.

In one embodiment, a library described herein comprises at least 2, 3, 4, 5, 6, 7, 8, 9, 10, 11, 12, 13, 14, 15, 16, 17, 18, 19, 20, 21, 22, 23, 24, 25, or more control plasmids or expression vectors. Controls are used herein to determine that the cell or in vivo system is functioning appropriately, thus validating the readout for unique regulatory elements. Controls promoters are additionally used to validate measuring approaches, e.g., PCR amplification of the synthetic nucleic acid. As discussed herein below, PCR amplification of a URE can result in non-uniform amplification, resulting in artifact expression frequency. Amplification of UMI tags can be used to control for this. Control promoters are also used as comparators to determine the strength of UREs in driving expression of the ORF. Exemplary control promoters include, but are not limited to, CMV-IE, CMVmp, EF1a, SV40, PL1, CBA and PGK. It is preferred that a control promoter is well characterized and has ubiquitous expression.

VI. Plurality of Cells

One aspect provided herein is a population of at least 50 cells expressing the plurality of synthetic nucleic acids described herein, or the library of expression vectors or library of plasmids described herein, such that the population of cells express the synthetic nucleic acids. Methods described herein utilize viral vectors to identify the strength of a URE in vitro and in vivo.

One skilled in the art can use standard technique to introduce the plurality of synthetic nucleic acids or the libraries of expression vectors or plasmids into the cell, such that the cell expresses said synthetic nucleic acids or libraries. These techniques include, but are not limited to transfection, lipofection, electroporation, transductions, and the like. One skilled in the art can assess whether a cell expresses the synthetic nucleic acid or the libraries of expression vectors or plasmids via, e.g., measuring the mRNA or protein levels of the synthetic nucleic acid by PCR-based assays or western blotting, imaging, biochemical assays, colorimetric assays, immunoassays, luciferase assay to name a few.

A cell can have stable expression the synthetic nucleic acid, or the libraries of expression vectors or plasmids. Such stable expression would result in the cell's progeny expressing the same. Alternatively, the cell can have transient expression of the synthetic nucleic acid, or the libraries of expression vectors or plasmids. Transient expression of a heterologous nucleic acid is not propagated in the progeny of the cell.

In one embodiment, the population of cells comprises at least 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, or more cells.

A cell can be, e.g., a eukaryotic, prokaryotic, bacterial, or viral cell. In one embodiment, the cell is a mammalian cell, e.g., a human cell. A cell can be derived from any origin, e.g., any tissue or organ, without limitation.

VIII. Identifying Strength of URE

Various aspects described herein provide methods for identifying the strength of a URE in vitro and in vivo. In general, the method includes expressing a synthetic nucleic acid in a cell using various means (e.g., via expression vector, plasmid, viral vector, etc.) such that the URE, transcribable reporter sequence, e.g., ORF, and plurality of barcodes unique to the specific URE are expressed in the cell. Next, mRNA is extracted from the cell and cDNA is synthesized from this template mRNA. The region of the synthetic nucleic acid comprising the URE, ORF, and plurality of unique barcodes is amplified and the resulting amplicon is analyzed via sequencing to reveal the abundance, e.g., frequency, of the barcode in the amplicon. The abundance of the barcode in the amplicon (barcode output) is normalized to each unique barcode content (barcode input) before expression to determine the expression frequency of the barcode, and thereby assessing the strength of the associated URE.

One aspect of the invention provides a method of identifying the strength of one or more unique regulatory elements (URE) having conformational effect on a ORF comprising (a) expressing a plurality of synthetic nucleic acids in a population of cells, the plurality of synthetic nucleic acids comprises (1) a first plurality of synthetic nucleic acids each comprising a unique regulatory element (URE) where the URE comprises (i) a nucleic acid sequence containing at least one discrete regulatory element (DRE), wherein the DRE is a control (or wild type) continuous nucleic acid sequence or a control discontinuous nucleic acid sequence associated with a plurality of unique barcodes corresponding with the at least one DRE, wherein each barcode is between 12-35 nucleotides in length and has a GC content between 25-65%; and (ii) the DRE is conformationally positioned in a preselected manner relative to a nucleic acid encoding a ORF, wherein if the URE does not contain a promoter, a separate promoter is operatively linked to the ORF; and (2) a second plurality of synthetic nucleic acids comprising a URE that further comprises a change in the conformation of said at least one DRE of a(1)(ii) relative to the ORF wherein the conformationally changed DRE is associated with a plurality of unique barcodes different than in (1)(i), wherein each barcode is between 12-35 nucleotides in length and has a GC content between 25-65%; (b) determining the expression frequency of each of the plurality of corresponding barcodes in (a)(1) and (a)(2); and (c) changing in a predetermined manner the conformation of at least one of the corresponding plurality of synthetic nucleic acids' DRE relative to the ORF; (d) determining the expression frequency of the at least one corresponding plurality of (c); and (e) comparing the expression frequency of (a)(1) and (a)(2) to determine the effect of the conformation change on the ORF expression.

Another aspect provides a method of identifying the strength of one or more unique regulatory elements (URE) having conformational effect on a ORF comprising (a) providing a plurality of synthetic nucleic acids, wherein the plurality of synthetic nucleic acid comprises (1) a first plurality of synthetic nucleic acids each comprising a unique regulatory element (URE), wherein the URE comprises (i) a nucleic acid sequence containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is conformationally positioned in a preselected manner relative to a nucleic acid encoding a ORF operatively linked to a promoter; wherein if the URE does not contain a promoter, a separate promoter is operatively linked to the ORF; and (2) a second plurality of synthetic nucleic acids comprising a URE further comprising a change in the conformation of said at least one DRE of a(1)(ii) relative to the ORF wherein the conformationally changed DRE is associated with a plurality of unique barcodes different than in (1)(i), wherein each barcode is between 12-35 nucleotides in length and has a GC content between 25-65%; (b) generating a library of plasmids or expression vectors by inserting the plurality of synthetic nucleic acids into a plurality of plasmids or expression vectors, wherein each resulting plasmid or expression vector comprises a single synthetic nucleic acid; (c) introducing the library of plasmids or expression vectors of step (b) into a population of cells; (d) determining the expression frequency of each of the plurality of corresponding barcodes in (a) (1) and (a) (2); and (e) comparing the expression frequency of (a)(1) and (a)(2) to determine the effect of the conformation change on the ORF expression.

Another aspect provides a method of identifying the strength of one or more unique regulatory elements (URE) having conformational effect on a ORF comprising (a) providing the plurality of synthetic nucleic acids, wherein the plurality of synthetic nucleic acid comprises (1) a unique regulatory element (URE), wherein the URE comprises (i) a first plurality of synthetic nucleic acid sequences each containing at least one discrete regulatory element (DRE), wherein the DRE is a control (or wild type) continuous nucleic acid sequence or a discontinuous nucleic acid sequence; (ii) associated with a plurality of unique barcodes corresponding with the at least one DRE, wherein each barcode is between 12-35 nucleotides in length and has a GC content between 25-65%; and the DRE is positioned in a preselected manner relative to a nucleic acid encoding a ORF operatively linked to a promoter; wherein if the URE does not contain a promoter, a separate promoter is operatively linked to the ORF; and (2) a second plurality of synthetic nucleic acids comprising a URE further comprising a change in the conformation of said at least one DRE of a(1)(ii) relative to the ORF, wherein the conformationally changed DRE is associated with a plurality of unique barcodes different than in (1)(i), wherein each barcode is between 12-35 nucleotides in length and has a GC content between 25-65%; (b) generating a library of plasmids or expression vectors by inserting the plurality of synthetic nucleic acids into a plurality of plasmids or expression vectors, wherein each resulting plasmid or expression vector comprises a single synthetic nucleic acid; (c) introducing the library of plasmids or expression vectors of step (b) into a viral vector such as HIV or an AAV vector to form an AAV vector library; (d) introducing the vector library into a population of cells; (e) determining the expression frequency of each of the corresponding barcodes of (a)(1) and (a)(2); and (f) comparing the expression frequency of (a)(1) and (a)(2) to determine the effect of the conformation change on the strength of expression.

a. Identifying Strength of URE In Vivo

One aspect provides a method of identifying the strength of a URE from a plurality of UREs in vivo, the method comprising (a) administering any of the populations of viral vectors described herein in vivo; and (b) determining the expression frequency of each of the plurality of barcodes, wherein the expression frequency of each of the plurality of barcodes is an indicator of the strength of the associated URE.

Another aspect provides a method of identifying the strength of a URE from a plurality of UREs, the method comprising (a) providing any of the pluralities of synthetic nucleic acids described herein; (b) inserting the plurality of synthetic nucleic acids into a library of plasmids or expression vectors, wherein the resulting plasmid or expression vector each comprise a single synthetic nucleic acid; (c) introducing the plurality of plasmids or expression vectors of step (b) into an viral vector; (d) administering the resulting viral vector of step (c) in vivo; and (d) determining the expression frequency of each of the plurality of barcodes, wherein the expression frequency of each of the plurality of barcodes is an indicator of the strength of the associated URE.

In various embodiments, the method further comprises the step of, after administering, waiting a sufficient amount of time for expression of the synthetic nucleic acids, the plasmids, or the expression vectors. In one embodiment, determining occurs at least 4 weeks post administration.

B. Identifying Strength of URE In Vitro

Another aspect described herein provides a method of identifying the strength of a URE from a plurality of UREs in vitro, the method comprising (a) expressing any of the plurality of synthetic nucleic acids described herein, any of the libraries of plasmids described herein, or any of the libraries of expression vectors described herein in a population of cells; and (b) determining the expression frequency of each of the plurality of barcodes, wherein the expression frequency of each of the plurality of barcodes is an indicator of the strength of the associated URE.

Another aspect described herein provides a method of identifying the strength of a URE from a plurality of UREs in vitro, the method comprising (a) providing any of the plurality of synthetic nucleic acids described herein; (b) inserting the plurality of synthetic nucleic acids into a library of plasmids or expression vectors, wherein the resulting plasmid or expression vector each comprise at least one DRE, an open reading frame, a viral vector TR or at least one partial viral vector comprising at least a part of a TR, and a plurality of barcodes associated with at least one DRE; (c) introducing the library of plasmids or expression vectors of step (b) into a population of cells; and (d) determining the expression frequency of the plurality of barcodes, wherein the expression frequency of each of the plurality of barcodes is an indicator of strength of the URE.

Another aspect described herein provides a method of identifying the strength of a URE from a plurality of UREs in vitro, the method comprising (a) providing any of the pluralities of synthetic nucleic acids described herein; inserting the plurality of synthetic nucleic acids into a library of plasmids or expression vectors, wherein the resulting plasmid or expression vector each comprise at least one DRE, an open reading frame, a viral vector TR or at least one partial viral vector comprising at least a part of a TR, and a plurality of barcodes associated with the at least one DRE; (b) introducing the plurality of plasmids or expression vectors of step (a) into a viral vector such as an AAV vector to form an AAV vector library; (c) introducing the AAV vector library into a population of cells; and (d) determining the expression frequency of the plurality of barcodes, wherein the expression frequency of each of the plurality of barcodes is an indicator of the strength of the URE.

In one embodiment, the method further comprises the step, after introducing, waiting a sufficient amount of time for expression of the synthetic nucleic acids, the plasmids, or the expression vectors. In one embodiment, determining occurs at least 24 or at least 48 hours post introducing the library of plasmids or expression vectors into an AAV vector or introducing AAV vector library to cell.

C. Determining Strength of a URE

In one embodiment, determining the expression frequency of the barcode unique to a specific URE includes the steps of: (a) obtaining mRNA from the population of cells or the population of AAV vectors; (b) synthesizing cDNA from the mRNA of step (a); (c) amplifying a region of nucleic acids (amplicon) from the cDNA of step (b); and (d) measuring the expression frequency of the plurality of barcodes in the amplicon of step (c).

In one embodiment, determining the expression frequency includes the steps of: obtaining mRNA from tissues or cells of interest after in vivo administration of viral vectors; synthesizing cDNA from the mRNA of step (a); amplifying a region of nucleic acids (amplicon) from the cDNA of step (b); and measuring the expression frequency of each of the plurality of barcodes in the amplicon of step (c).

mRNA can be extracted from, e.g., a cell expressing the synthetic nucleic acid using standard techniques known in the art. For example, mRNA extraction kits are readily available from commercial sources, e.g., Millipore Sigma, product number 11741985001, and ThermoFisher catalog number 61006. One skilled in the art will be capable of synthesizing complementary DNA (cDNA) is from the extracted mRNA using standard techniques in the art. For example, cDNA is reverse transcribed using mRNA as template. Reverse transcriptases (RTs) use the mRNA template and a short primer complementary to the 3′ end of the mRNA to direct the synthesis of the first strand cDNA, which can be used directly as a template for the Polymerase Chain Reaction (PCR). Alternatively, the first-strand cDNA can be made double-stranded using DNA Polymerase I and DNA Ligase.

Tissues and cells expressing a synthetic nucleic acid described herein can be extracted from the in vivo system using standard techniques. For example, a mouse that has been administered an AAV vector or any other expression vector carrying the synthetic nucleic acid can be euthanized and organs, tissues, or cells samples can be isolated and harvested using standard approaches. For example, an organ or tissue can be homogenized prior to mRNA extraction using standard methods, e.g., as described above.

Following synthesis of cDNA, the region containing the plurality of barcodes is amplified using primers specific for this region. This amplicon is produced, e.g., using standard PCR methods known in the art. It is preferred that a minimum number of PCR amplification rounds are used to prevent stochasticity bias (i.e., non-uniform amplification). In one embodiment, the synthetic nucleic acids comprising the barcodes are further modified to include UMI tags to further control for non-uniform amplification of the amplicon. In one embodiment, primers incorporate a gene specific part which binds to the URE template cDNA, the illumine barcode and adapter. For example, up to 24 different primers having different illumine indexes allowing multiplexing of the generated sequencing data are used. In one embodiment, primers allow efficient binding to the sequencing flowcell. In one embodiment, the left primer (leftBC) has a sequence of CAAGCAGAAGACGGCATACGAGATACGAGACTGATTAGTCAGTCAGCCCTCCGC CTTGCCCTGA (SEQ ID NO: 9), and the right primer (Right_UPAS) has a sequence of AATGATACGGCGACCACCGAGATCTACACTATGGTAATTGTTCCTACTATTCCGT ACCGTAGGGT (SEQ ID NO: 10).

In one embodiment, measuring is performed by sequencing. Exemplary sequencing methods include, but are not limited to, Sanger sequencing methods, high throughput sequencing methods, and next generation sequencing (e.g., Illumina® sequencing).

The expression frequency of a given unique barcode or a plurality of unique barcodes is an indicator of the strength of the associated unique regulatory element. To determine the expression frequency of a barcode, the barcode output is normalized to the barcode input. As described herein, “barcode output” is the frequency of a given barcode in an amplicon as measured by, e.g., sequencing. As described herein, “barcode input” refers to each unique barcode content before expression. Barcode input is determined prior to expression of the barcode in a given system, e.g., in a cell or in vivo system, and can be measured using sequencing methods. In one embodiment, expression above the baseline activity of the minimal promoter is defined as “active”. One skilled in the art can determine the activity of a regulatory element, e.g., by comparing the activity level of a given regulatory element to a reference promoter, such as non-tissue-specific promoter, CMV-IE, or liver specific promoters, LP1 or TBG.

Accordingly, in a further aspect, the present invention provides a method for producing an expression product, the method comprising: a) providing a population of eukaryotic cells with any plurality of synthetic nucleic acids according to the present invention, where the open reading frame comprises a nucleic acid sequence encoding an expression product, and incubating said population of cells under suitable conditions for production of the expression product; and isolating the expression product from said population of cells. Further optional and preferred features of methods for producing an expression product are discussed herein for the other aspects of the invention, and these apply to the present aspect mutatis mutandis. In some embodiments, the expression product is a therapeutic protein or a toxic protein.

Accordingly, a further aspect of the invention provides a pharmaceutical composition comprising a nucleic acid expression construct or a vector comprising a synthetic nucleic acid as disclosed herein, the synthetic nucleic acid comprising a URE, an open reading frame and a plurality of unique barcodes, where the open reading frame comprises a nucleic acid sequence encoding an expression product, and wherein the expression product is a therapeutic protein or a toxic protein. Further optional and preferred features of pharmaceutical composition are discussed above for the other aspects of the invention, and these apply to the present aspect mutatis mutandis.

In a further aspect of the invention there is provided the use of nucleic acid expression constructs and vectors comprising a synthetic nucleic acid as disclosed herein, the synthetic nucleic acid comprising a URE, an open reading frame and a plurality of barcodes unique to the URE, where the open reading frame comprises a nucleic acid sequence encoding an expression product, and wherein the expression product is a therapeutic protein or a toxic protein, for the manufacture of a pharmaceutical composition.

Another further aspect of the present invention relates to a cell comprising a synthetic nucleic acid expression construct or vector comprising a synthetic nucleic acid as disclosed herein, the synthetic nucleic acid comprising a URE, an open reading frame and a plurality of barcodes unique to said URE, where the open reading frame comprises a nucleic acid sequence encoding an expression product, and wherein the expression product is a therapeutic protein or a toxic protein. Further optional and preferred features of such cells are discussed above for the other aspects of the invention, and these apply to the present aspect mutatis mutandis. In a further aspect, the invention provides the nucleic acid expression constructs, vectors, cells or pharmaceutical compositions comprising a synthetic nucleic acid as disclosed herein, the synthetic nucleic acid comprising a URE, an open reading frame and a unique barcode, where the open reading frame comprises a nucleic acid sequence encoding an expression product, and wherein the expression product is a therapeutic protein or a toxic protein according to the present invention for use in a method of treatment or therapy. Further optional and preferred features of such methods are discussed above for the other aspects of the invention, and these apply to the present aspect mutatis mutandis.

Use of AAV Vector in Gene Therapy to Screen in Animal Models

In one embodiment, the library complexity is determined by the volume of vector, e.g., AAV vector to be injected in the subject.

In one embodiment, all promoter inserts are the same size, or are essentially the same size.

In one embodiment, complex libraries are made in normal plasmids before being sub-cloned into the pAAV backbone. It was previously found that directly cloning the library into a pAAV results in a low complexity library due to the inefficiency introduced by the ITRs. It was found that there is incompatibility of methods 37 C vs 32 C for all nonT4 methods vs ITR.

In one embodiment, the methods described herein utilize single stranded AAV. In an alternative embodiment, the methods described herein utilize self-complementary AAV (scAAV). The use of scAAV removes potential problem of concatamerisation messing up barcode quantification step where distal enhancer elements may influence barcodes associated with different promoters

In one embodiment, representation of E. coli library transformation is maintained across a complex library by increasing number of colony forming units.

In one embodiment, an amplicon is prepared using full Illumina tags to avoid PCR bias in library preparation. In one embodiment, UMI tags are introduced to the vector to reduce stochasticity during amplicon generation.

In one embodiment, barcodes are analyzed from cDNA or AAV genome, or AAV preparation to allow for calculating barcode frequency and/or promoter strength.

In various embodiment, barcode controls are used to show functionality of method, gauge promoter expression strength, and/or to verify that there is no enhancer crosstalk or interference with candidate promoters and/or enhancers.

Examining Structural, Conformational and Distance Relationship Between ITRs and Promoter Parts

In one embodiment, tiling for different mutations in the ITR (e.g. a deletion, substitution, or addition in the ITR, such as the holiday junction or loop region) or the sequence spanning between the ITR and the promoter allows for conformation analysis, i.e. determining key sequences of importance (e.g., in the ITR or in the sequence spanning between the ITR and the promoter) that may influence promoter activity.

In one embodiment, the methods described herein assess the relationship of the distance between the promoter from the ITR. This allows for screening a group of standard promoters in the art with varying distances from the ITRs.

In one embodiment, the methods described herein assess how ITR mutations (e.g., a deletion, substitution, or addition in the ITR) effects promoter activity and identify essential promoter-ITR interaction. In one embodiment, methods described herein can be used in any known cell type to determine if the identified promoter-ITR interaction is cell-type specific.

In one embodiment, the methods described herein screen for effects of hybrid ITRs on promoter activity.

In one embodiment, the methods described herein screen for effects of ITRs from different serotypes on promoter activity.

In one embodiment, any of the vectors described herein (e.g., comprising any of the UREs described herein) further comprise stuffer fragment to achieve optimal and equal packaging size. In one embodiment, the stuffer fragment is introduced on the 3′ end, and not the 5′ end, to reduce interference with the test promoter.

In one embodiment, the backbone of any of the vectors described herein (e.g., comprising any of the UREs described herein) is increased in size to decreasing non-specific packaging. For example, the backbone is increased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more as compared to a wild-type backbone.

In one embodiment, any sequence, e.g., a promoter sequence, a barcode, an ORF, or the like, is inserted into an insulator sequence, to reduce potential interference of ITRs to test promoter.

In one embodiment, generation of high throughput data from methods described herein allow the creation of algorithms to predict promoter-ITR interactions, structural and conformational changes. High throughput data can be used in, e.g., machine learning systems.

One can use any of the above methods in multiple combinations and fall within the scope of this invention.

The invention described herein can further be described in the following numbered paragraphs:
1. A method of identifying the strength of one or more unique regulatory elements (URE) having conformational effect on an transcribable reporter sequence, e.g., ORF, comprising:
   a. expressing a plurality of synthetic nucleic acids in a population of cells, the plurality of synthetic nucleic acids comprises:
      1. a first plurality of synthetic nucleic acids each comprising a unique regulatory element (URE) where the URE comprises:
         i. a nucleic acid sequence containing at least one discrete regulatory element (DRE), wherein the DRE is a control (or wild type) continuous nucleic acid sequence or a control discontinuous nucleic acid sequence associated with a plurality of unique barcodes corresponding with the at least one DRE, wherein each barcode is between 12-35 nucleotides in length and has a GC content between 25-65%; and
         ii. the DRE is conformationally positioned in a preselected manner relative to a nucleic acid encoding an transcribable reporter sequence, wherein if the URE does not contain a promoter, a separate promoter is operatively linked to the transcribable reporter sequence; and
      2. a second plurality of synthetic nucleic acids comprising a URE that further comprises a change in the conformation of said at least one DRE of a(1)(ii) relative to the ORF wherein the conformationally changed DRE is associated with a plurality of unique barcodes different than in (1)(i), wherein each barcode is between 12-35 nucleotides in length and has a GC content between 25-65%;
   b. determining the expression frequency of each of the plurality of corresponding barcodes in (a)(1) and (a)(2); and
   c. changing in a predetermined manner the conformation of at least one of the corresponding plurality of synthetic nucleic acids' DRE relative to the transcribable reporter sequence;
   d. determining the expression frequency of the at least one corresponding plurality of (c); and
   e. comparing the expression frequency of (a)(1) and (a)(2) to determine the effect of the conformation change on the transcribable reporter sequence expression.
2. The method of paragraph 1, wherein the plurality of synthetic nucleic acids is expressed in a population of cells using a population of viral vectors.
3. The method of any preceding paragraph, wherein the DRE is proximal to or within a Holliday junction and a change in at least one of the Holliday junctions is made.
4. The method of any preceding paragraph, wherein the change in conformation is made by the addition, deletion, or substitution of one or more nucleic acids.
5. The method of any preceding paragraph, wherein at least one DRE is present in a terminal repeat (TR).
6. The method of any preceding paragraph, wherein the viral vector is a parvovirus, a lentivirus, or an adenovirus.
7. The method of any preceding paragraph, wherein the parvovirus is a dependovirus and the change in conformation is in at least one of the A, A′, B, B′, C, or C′ loops.
8. The method of any preceding paragraph, wherein the parvovirus is an adeno-associated virus (AAV) and the change in conformational is in at least one of the A, A′, B, B′, C, C′, D, D′ regions.
9. The method of any preceding paragraph, wherein the viral vector is a lentiviral vector, the DRE is TAT, and the conformational change is made in the TAR RNA stem.
10. The method of any preceding paragraph, wherein the viral vector is a lentiviral vector, the DRE is TAT, and the conformational change is made in the UU-rich bulge.
11. The method of any preceding paragraph, wherein the viral vector is a lentiviral vector, the DRE is REV, a REV Responsive Element (RRE) is present in the nucleic acid, and the conformational change is made in the RRE.
12. The method of any preceding paragraph, wherein the DRE is proximal to or within the conformation change.
13. The method of any preceding paragraph, wherein the conformational change occurs by the addition, substitution, or deletion of at least one nucleic acid.
14. The method of any preceding paragraph, wherein the addition, substitution, or deletion results in a Holliday junction.
15. The method of any preceding paragraph, wherein the plurality of synthetic nucleic acids is expressed in a population of cells in vitro using a population of AAV vectors.
16. The method of any preceding paragraph, wherein the plurality of synthetic nucleic acids is expressed in a population of cells in vivo using a population of AAV vectors.
17. A method of identifying the strength of one or more unique regulatory elements (URE) having conformational effect on an transcribable reporter sequence comprising:
   a. providing a plurality of synthetic nucleic acids, wherein the plurality of synthetic nucleic acid comprises:
      1. a first plurality of synthetic nucleic acids each comprising a unique regulatory element (URE), wherein the URE comprises:
         i. a nucleic acid sequence containing at least one discrete regulatory element (DRE), wherein the DRE is a control (or wild type) continuous nucleic acid sequence or a discontinuous nucleic acid sequence;
         ii. associated with a plurality of unique barcodes corresponding with the at least one DRE, wherein each barcode is between 12-35 nucleotides in length and has a GC content between 25-65%; and the DRE is conformationally positioned in a preselected manner relative to a nucleic acid encoding an transcribable reporter sequence operatively linked to a promoter; wherein if the URE does not contain a promoter, a separate promoter is operatively linked to the transcribable reporter sequence; and
      2. a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%;
   b. generating a library of plasmids or expression vectors by inserting the plurality of synthetic nucleic acids into a plurality of plasmids or expression vectors, wherein each resulting plasmid or expression vector comprises a single synthetic nucleic acid;
   c. introducing the library of plasmids or expression vectors of step (b) into a population of cells;
   d. determining the expression frequency of each of the plurality of corresponding barcodes in (a) (1) and (a) (2); and
   e. comparing the expression frequency of (a)(1) and (a)(2) to determine the effect of the conformation change on the transcribable reporter sequence expression.
18. A method of identifying the strength of one or more unique regulatory elements (URE) having conformational effect on an transcribable reporter sequence comprising:
   a. providing the plurality of synthetic nucleic acids, wherein the plurality of synthetic nucleic acid comprises:
      1. a unique regulatory element (URE), wherein the URE comprises:
         i. a first plurality of synthetic nucleic acid sequences each containing at least one discrete regulatory element (DRE), wherein the DRE is a control (or wild type) continuous nucleic acid sequence or a discontinuous nucleic acid sequence;
         ii. associated with a plurality of unique barcodes corresponding with the at least one DRE, wherein each barcode is between 12-35 nucleotides in length and has a GC content between 25-65%; and the DRE is positioned in a preselected manner relative to a nucleic acid encoding an transcribable reporter sequence operatively linked to a promoter; wherein if the URE does not contain a promoter, a separate promoter is operatively linked to the transcribable reporter sequence; and
      2. a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%;
   b. generating a library of plasmids or expression vectors by inserting the plurality of synthetic nucleic acids into a plurality of plasmids or expression vectors, wherein each resulting plasmid or expression vector comprises a single synthetic nucleic acid;
   c. introducing the library of plasmids or expression vectors of step (b) into an AAV vector to form an AAV vector library;
   d. introducing the AAV vector library into a population of cells;
   e. determining the expression frequency of each of the corresponding barcodes of (a)(1) and (a)(2)
   f. comparing the expression frequency of (a)(1) and (a)(2) to determine the effect of the conformation change on the strength of expression.
19. The method of any preceding paragraph, further comprising the step of, after step (a), waiting a sufficient amount of time for expression of the plurality of synthetic nucleic acids in the population of cells.
20. The method of any preceding paragraph, further comprising the step of, after step (c), waiting a sufficient amount of time for expression of the library of plasmids or expression vectors of step (b).
21. The method of any preceding paragraph, wherein determining includes the steps of:
   a. obtaining mRNA from the population of cells;
   b. synthesizing cDNA from the mRNA of step (a);
   c. amplifying a region of nucleic acids (amplicon) from the cDNA of step (b); and
   d. measuring the expression frequency of each of the plurality of barcodes in the amplicon of step (c).
22. The method of any preceding paragraph, wherein measuring is performed by sequencing.
23. The method of any preceding paragraph, wherein the expression frequency of each of the plurality of barcodes is the normalized to a barcode input, and wherein the barcode input is each unique barcode content before expression.
24. The method of any preceding paragraph, wherein the expression frequency of the barcode measured in the amplicon is a barcode output.
25. The method of any preceding paragraph, wherein at least one DRE is a discontinuous DRE.
26. The method of any preceding paragraph, wherein the discontinuous DRE comprises a portion of the DRE located 5′ of the transcribable reporter sequence, and a portion of the DRE located 3′ of the transcribable reporter sequence.
27. The method of any preceding paragraph, wherein the discontinuous DRE comprises a non-DRE nucleic acid sequence located in a 5′- or 3′-portion of the DRE.
28. The method of any preceding paragraph, wherein the at least one DRE is located within 200-500 bp of the at least one TR, or portion thereof.
29. The method of any preceding paragraph, wherein the at least one DRE is located within 20-200 bp of the at least one TR, or portion thereof.
30. The method of any preceding paragraph, wherein the at least one DRE is located within 20 bp of the at least one TR, or portion thereof.
31. The method of any preceding paragraph, wherein the URE strength is measured in the same system from which it is derived.
32. The method of any preceding paragraph, wherein at least part of the at least one discontinuous DRE includes a TR.
33. The method of any preceding paragraph, wherein the at least one TR, or portion thereof, comprises at least one modification.
34. The method of any preceding paragraph, wherein the at least one TR comprises at least 1, 2, 3, 4, 5, 6, or more modifications.
35. The method of any preceding paragraph, wherein the at least 1, 2, 3, 4, 5, 6, or more modifications are associated with the same plurality of unique barcodes as in any preceding paragraph.
36. The method of any preceding paragraph, wherein the synthetic nucleic acid contains at least 2, 3, 4, 5, 6, or more TRs, or portion thereof.
37. The method of any preceding paragraph, wherein the synthetic nucleic acid contains at least 2, 3, 4, 5, 6, or more discontinuous DREs.
38. The method of any preceding paragraph, wherein the URE comprises at least DRE selected from the group consisting of: a promoter, a transcription factor binding site, an enhancer, a silencer, a boundary control element, an insulator, a locus control region, a response element, a binding site, a segment of a terminal repeat, a responsive site, a stabilizing element, a de-stabilizing element, and a splicing element.
39. The method of any preceding paragraph, wherein the nucleic acid sequence containing at least one DRE comprises a combination of DREs.
40. The method of any preceding paragraph, wherein the combination of DREs contain at least 2, 3, 4, 5, 6, or more regulatory sequence elements.
41. The method of any preceding paragraph, wherein the combination of DREs is associated with the same plurality of unique barcodes of any preceding paragraph.
42. The method of any preceding paragraph, wherein the viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector
43. The method of any preceding paragraph, wherein the AAV vector is a AAV serotype selected from the group consisting of: 1, 2, 3a, 3b, 4, 5, 6, 7, 8, 9, 10, 11, and 13.
44. The method of any preceding paragraph, wherein the synthetic nucleic acid comprises an inverted terminal repeat (ITR), or a portion thereof.
45. The method of any preceding paragraph, wherein the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a TRS (terminal resolution site), and a Rep binding site (RBS).
46. The method of any preceding paragraph, wherein the ITR is a wild-type inverted terminal repeat (ITR), a mutant ITR, or a synthetic ITR, wherein the mutant or synthetic ITR comprises a modification as compared to the wild-type ITR sequence.
47. The method of any preceding paragraph, wherein the A region, A′ region, B region, B′ region, C region, C′ region, D region, or D′ region is derived from a wild-type inverted terminal repeat (ITR), a mutant ITR, a truncated ITR, or a synthetic ITR.
48. The method of any preceding paragraph, wherein the TR is a long terminal repeat (LTR), or a portion thereof.
49. The method of any preceding paragraph, wherein the modification is a base pair insertion, deletion, mutation, truncation, or substitution as compared to the wild-type ITR sequence.
50. The method of any preceding paragraph, wherein the at least one DRE and the TR sequence are separated by 1-500 base pairs.
51. The method of any preceding paragraph, wherein each portion of a discontinuous DRE (dcDRE) is separated by 1-500 base pairs.
52. The method of any preceding paragraph, wherein each portion of a discontinuous DRE (dcDRE) is separated by at least 50 base pairs.
53. The method of any preceding paragraph, wherein one portion of a discontinuous DRE (dcDRE) can be 5′ of the transcribable reporter sequence, and a second portion of the dcDRE is 3′ of the transcribable reporter sequence.
54. The method of any preceding paragraph, wherein the transcribable reporter sequence is the ORF of a marker gene.
55. The method of any preceding paragraph, wherein the marker gene encodes a fluorescent protein, a luminescent protein, or an element tag.
56. The method of any preceding paragraph, wherein the barcode contains at least one of each: adenine, thymine, guanine, and cytosine.
57. The method of any preceding paragraph, wherein the barcode is a semi-degenerate barcode.
58. The method of any preceding paragraph, wherein the barcode does not contain tracts of more than three homopolymers in succession.
59. The method of any preceding paragraph, wherein the barcode does not contain the nucleic acid sequence of a restriction enzyme.
60. The method of any preceding paragraph, wherein the barcode has a hamming distance greater than 2.
61. The method of any preceding paragraph, wherein the barcode is between 12-25 nucleotides in length.
62. The method of any preceding paragraph, wherein the barcode is between 12-28 nucleotides in length.
63. The method of any preceding paragraph, wherein the barcode has a complexity of at least 4.3×10⁷, at least 2.7×10⁸, or at least 1×10¹².
64. The method of any preceding paragraph, wherein a plurality of barcodes comprises at least 2, 3, 4, 5, 6, 7, 8, 9, 10, or more barcodes.
65. The method of any preceding paragraph, wherein a plurality of barcodes comprises 2-20 barcodes.
66. The method of any preceding paragraph, wherein the synthetic nucleic acid is further modified for next generation sequencing.
67. The method of any preceding paragraph, wherein the synthetic nucleic acid comprises at least one unique molecular identifier (UMI) and at least one unique primer annealing sites (UPAS) tag.
68. A plurality of at least 50 synthetic nucleic acids, each synthetic nucleic acid comprising a URE, where the URE comprises:
   a. a nucleic acid sequence containing at least one discrete regulatory element (DRE), wherein the DRE is a continuous nucleic acid sequence or a discontinuous nucleic acid sequence;
   b. a nucleic acid sequence encoding an open reading frame;
   c. a nucleic acid sequence encoding a viral vector terminal repeat (TR); and
   d. a plurality of unique barcodes associated with the at least one DRE,
   wherein each barcode has a GC content between 25-65%.
69. A plurality of at least 50 synthetic nucleic acids, each synthetic nucleic acid comprising a URE, where the URE comprises:
   a. a nucleic acid sequence containing at least one discrete regulatory element (DRE), wherein the DRE is a continuous nucleic acid sequence or a discontinuous nucleic acid sequence;
   b. a nucleic acid sequence encoding an open reading frame;
   c. a nucleic acid sequence encoding at least one partial viral vector comprising at least a part of a terminal repeat (TR); and
   d. a plurality of unique barcodes associated with the at least one DRE,
   wherein each barcode is between 12-35 nucleotides in length and have a GC content between 25-65%.
70. The plurality of synthetic nucleic acids of any preceding paragraph, wherein the DRE comprises at least one regulatory sequence element selected from the group consisting of: a promoter, a transcription factor binding site, an enhancer, a silencer, a boundary control element, an insulator, a locus control region, a response element, a binding site, a segment of a terminal repeat, a responsive site, a stabilizing element, a de-stabilizing element, and a splicing element.
71. The plurality of synthetic nucleic acids of any preceding paragraph, wherein the nucleic acid sequence containing at least one DRE comprises a combination of DREs.
72. The plurality of synthetic nucleic acids of any preceding paragraph, wherein the combination of DREs contain 2-6 DREs.
73. The plurality of synthetic nucleic acids of any preceding paragraph, wherein the combination of regulatory sequence elements is associated with the same plurality of unique barcodes of any preceding paragraph.
74. The plurality of synthetic nucleic acids of any preceding paragraph, wherein at least part of the at least one DRE includes a TR.
75. The plurality of synthetic nucleic acids of any preceding paragraph, wherein the synthetic nucleic acid contains at least 2 TRs.
76. The plurality of synthetic nucleic acids of any preceding paragraph, wherein the at least one discontinuous regulatory element comprises at least one modification.
77. The plurality of synthetic nucleic acids of any preceding paragraph, wherein the viral vector comprises at least 4 modifications.
78. The plurality of synthetic nucleic acids of any preceding paragraph, wherein the viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector
79. The plurality of synthetic nucleic acids of any preceding paragraph, wherein the AAV vector is a AAV serotype selected from the group consisting of: 1, 2, 3a, 3b, 4, 5, 6, 7, 8, 9, 10, 11, and 13.
80. The plurality of synthetic nucleic acids of any preceding paragraph, wherein the TR is an inverted terminal repeat (ITR).
81. The plurality of synthetic nucleic acids of any preceding paragraph, wherein the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a spacer sequence, a CAP gene sequence, a Rep gene sequence, a Rep Binding Site, and a terminal resolution site.
82. The plurality of synthetic nucleic acids of any preceding paragraph, wherein the ITR is a wild-type inverted terminal repeat (ITR), a mutant ITR, or a synthetic ITR
83. The plurality of synthetic nucleic acids of any preceding paragraph, wherein the A region, A′ region, B region, B′ region, C region, C′ region, D region, or D′ region is derived from a wild-type inverted terminal repeat (ITR), a mutant ITR, a truncated ITR, or a synthetic ITR.
84. The plurality of synthetic nucleic acids of any preceding paragraph, wherein the TR is a long terminal repeat (LTR).
85. The plurality of synthetic nucleic acids of any preceding paragraph, wherein the modification is a base pair insertion, deletion, mutation, truncation, or substitution as compared to the wild-type sequence.
86. The plurality of synthetic nucleic acids of any preceding paragraph, wherein the DRE and the TR comprised in the viral vector or the partial vector are separated by 2-500 base pairs.
87. The plurality of synthetic nucleic acids of any preceding paragraph, wherein the DREs are separated by 2-200 base pairs.
88. The plurality of synthetic nucleic acids of any preceding paragraph, wherein the open reading frame is the open reading frame of a marker gene.
89. The plurality of synthetic nucleic acids of any preceding paragraph, wherein the marker gene encodes a fluorescent protein, a luminescent protein, or an element tag.
90. The plurality of synthetic nucleic acids of any preceding paragraph, wherein the barcode contains at least one of each: adenine, thymine, guanine, and cytosine.
91. The plurality of synthetic nucleic acids of any preceding paragraph, wherein the barcode is a semi-degenerate barcode.
92. The plurality of synthetic nucleic acids of any preceding paragraph, wherein the barcode does not contain tracts of more than three homopolymers in succession.
93. The plurality of synthetic nucleic acids of any preceding paragraph, wherein the barcode does not contain the nucleic acid sequence of a restriction enzyme.
94. The plurality of synthetic nucleic acids of any preceding paragraph, wherein the barcode has a hamming distance greater than 2.
95. The plurality of synthetic nucleic acids of any preceding paragraph, wherein the barcode is between 12-28 nucleotides in length.
96. The plurality of synthetic nucleic acids of any preceding paragraph, wherein the barcode is between 12-25 nucleotides in length.
97. The plurality of synthetic nucleic acids of any preceding paragraph, wherein the barcode has a complexity of at least 4.3×10⁷, at least 2.7×10⁸, or at least 1×10¹².
98. The plurality of synthetic nucleic acids of any preceding paragraph, wherein a plurality of barcodes comprises at least 2 barcodes.
99. The plurality of synthetic nucleic acids of any preceding paragraph, wherein a plurality of barcodes comprises at least 2, 3, 4, 5, 6, 7, 8, 9, 10, or more barcodes.
100. The plurality of synthetic nucleic acids of any preceding paragraph, wherein the synthetic nucleic acid is further modified for next generation sequencing.
101. The plurality of synthetic nucleic acids of any preceding paragraph, wherein the synthetic nucleic acid comprises at least one UMI and at least one UPAS.
102. A library of at least 50 plasmids expressing the plurality of synthetic nucleic acids of any preceding paragraph.
103. A library of at least 50 expression vectors comprising the plurality of synthetic nucleic acids of any preceding paragraph.
104. The library of any preceding paragraph, wherein the library comprises control plasmids or control expression vectors.
105. A population of cells comprising the library of any preceding paragraph.
106. The population of cells of any preceding paragraph, wherein the cells are eukaryotic, prokaryotic, viral, or bacterial.
107. The population of cells of any preceding paragraph, wherein the synthetic nucleic acids, plasmids, or expression vectors is transiently expressed.
108. The population of cells of any preceding paragraph, wherein the synthetic nucleic acids, plasmids, or expression vectors is stably expressed.
109. A population of at least 50 viral vectors expressing the plurality of synthetic nucleic acids of any preceding paragraph, the library of plasmids of any preceding paragraph, or the library of expression vectors of any preceding paragraph.
110. The population of viral vectors of any preceding paragraph, wherein the viral vector is an AAV vector.
111. A method of identifying the strength of a URE from a plurality of UREs in vitro, the method comprising:
   a. expressing the plurality of synthetic nucleic acids of any preceding paragraph, the library of plasmids of any preceding paragraph, or the library of expression vectors of any preceding paragraph, in a population of cells; and
   b. determining the expression frequency of each of the plurality of barcodes,
   wherein the expression frequency of each of the plurality of barcodes is an indicator of the strength of the associated URE.
112. A method of identifying the strength of a URE from a plurality of UREs in vitro, the method comprising:
   a. providing the plurality of synthetic nucleic acids of any preceding paragraph;
   b. inserting the plurality of synthetic nucleic acids into a library of plasmids or expression vectors, wherein the resulting plasmid or expression vector each comprise at least one DRE, an open reading frame, a viral vector terminal repeat (TR) or at least one partial viral vector comprising at least a part of a terminal repeat (TR), and a plurality of barcodes associated with at least one DRE;
   c. introducing the library of plasmids or expression vectors of step (b) into a population of cells; and
   d. determining the expression frequency of the plurality of barcodes,
   wherein the expression frequency of each of the plurality of barcodes is an indicator of strength of the URE.
113. A method of identifying the strength of a URE from a plurality of UREs in vitro, the method comprising:
   a. providing the plurality of synthetic nucleic acids of any preceding paragraph;
   b. inserting the plurality of synthetic nucleic acids into a library of plasmids or expression vectors, wherein the resulting plasmid or expression vector each comprise at least one DRE, an open reading frame, a viral vector terminal repeat (TR) or at least one partial viral vector comprising at least a part of a terminal repeat (TR), and a plurality of barcodes associated with the at least one DRE;
   c. introducing the plurality of plasmids or expression vectors of step (b) into an AAV vector to form AAV vector library;
   d. introducing the AAV vector library into a population of cells; and
   e. determining the expression frequency of the plurality of barcodes,
   wherein the expression frequency of each of the plurality of barcodes is an indicator of the strength of the URE.
114. The method of any of any preceding paragraph, further comprising the step of, after step (c) of any preceding paragraph or after step (d) of any preceding paragraph waiting a sufficient amount of time for expression of the synthetic nucleic acids, the plasmids, or the expression vectors.
115. The method of any of any preceding paragraph, wherein determining the expression frequency includes the steps of:
   a. obtaining mRNA from the population of cells;
   b. synthesizing cDNA from the mRNA of step (a);
   c. amplifying a region of nucleic acids (amplicon) from the cDNA of step (b); and
   d. measuring the expression frequency of each of the plurality of barcodes in the amplicon of step (c).
116. The method of any preceding paragraph, wherein measuring is performed by sequencing.
117. The method of any preceding paragraph, wherein is the expression frequency of the barcode measured in the amplicon is a barcode output.
118. The method of any preceding paragraph, wherein the barcode output is the normalized to a barcode input, and wherein the barcode input is each unique barcode content before expression.
119. A method of identifying the strength of a URE from a plurality of UREs in vivo, the method comprising:
   a. administering the population of viral vectors of any preceding paragraph in vivo; and
   b. determining the expression frequency of each of the plurality of barcodes,
   wherein the expression frequency of each of the plurality of barcodes is an indicator of the strength of the associated URE.
120. A method of identifying the strength of a URE from a plurality of UREs, the method comprising:
   a. providing the plurality of synthetic nucleic acids of any preceding paragraph;
   b. inserting the plurality of synthetic nucleic acids into a library of plasmids or expression vectors, wherein the resulting plasmid or expression vector each comprise a single synthetic nucleic acid;
   c. introducing the plurality of plasmids or expression vectors of step (b) into an viral vector;
   d. administering the resulting viral vector of step (c) in vivo; and
   e. determining the expression frequency of each of the plurality of barcodes,
   wherein the expression frequency of each of the plurality of barcodes is an indicator of the strength of the associated URE.
121. The method of any preceding paragraph, wherein the viral vector is an AAV vector.
122. The method of any preceding paragraph, further comprising the step of, after administering, waiting a sufficient amount of time for expression of the synthetic nucleic acids, the plasmids, or the expression vectors.
123. The method of any preceding paragraph, wherein determining the expression frequency includes the steps of:
   a. obtaining mRNA from tissues or cells of interest after in vivo administration of viral vectors;
   b. synthesizing cDNA from the mRNA of step (a);
   c. amplifying a region of nucleic acids (amplicon) from the cDNA of step (b); and
   d. measuring the expression frequency of each of the plurality of barcodes in the amplicon of step (c).
124. The method of any preceding paragraph, wherein measuring is performed by sequencing.
125. The method of any preceding paragraph, wherein is the expression frequency of the barcode measured in the amplicon is a barcode output.
126. The method any preceding paragraph, wherein the barcode output is normalized to a barcode input, and wherein the barcode input is each unique barcode content before expression.
127. The method of any of any preceding paragraph, wherein the URE strength is measured in the same system from which it is derived.
128. A plurality of at least 50 synthetic nucleic acids, each synthetic nucleic acid comprising:
   a. a nucleic acid sequence containing at least one discrete regulatory element (DRE);
   b. a nucleic acid sequence encoding an open reading frame;
   c. a nucleic acid sequence encoding a viral vector; and
   d. a plurality of unique barcodes associated with the at least one DRE,
   wherein each barcode is between 12-35 nucleotides in length and have a GC content between 25-65%.
129. A plurality of at least 50 synthetic nucleic acids, each synthetic nucleic acid comprising:
   a. a nucleic acid sequence containing at least one discrete regulatory element (DRE);
   b. a nucleic acid sequence encoding an open reading frame;
   c. a nucleic acid sequence encoding at least one partial viral vector; and
   d. a plurality of unique barcodes associated with the at least one DRE,
   wherein each barcode is between 12-35 nucleotides in length and have a GC content between 25-65%.
130. The plurality of synthetic nucleic acids of any preceding paragraph, wherein the viral vector comprises 1-6 modifications.
131. The plurality of synthetic nucleic acids of any preceding paragraph, wherein the 1-6 modifications are associated with the same plurality of unique barcodes of any preceding paragraph.
132. The plurality of synthetic nucleic acids of any preceding paragraph, wherein the partial viral vector is selected from the group consisting of: a terminal repeat, response element, cis-acting viral element, and a trans-acting viral element.
133. The method of any preceding paragraph, wherein the conformational change is not determined.
134. The method of any preceding paragraph, wherein the conformational change determined by assessing the at least one mutation against a non-altered sequence under the same condition.

### EXAMPLES

### Example 1

### Identification of Unique Regulatory Elements In Vitro

To effectively screen and identify the relationship between a promotor and the conformation of a vector, e.g., a viral vector, from libraries with a complexity of up to 1×10⁶ a high content screening (HCS) methodology had been established.

The HCS methodology described herein is outlined in FIG. 2. Briefly, a high complexity library of synthetic promoters is constructed from a discreet pool of transcription factor binding sites (TFBS). Each TFBS is represented by one or more positional weight matric (PWMs). These PWM are selected through their overrepresentation in highly active constitutively expressed target key genes and their proximity to the transcription start site. The selected PWM are randomly concatenated to form a complex library of synthetic promoter (SP) constructs. This library is size selected and integrated into (1) a screening vector comprising a wild-type ITR, and (2) a screening vector comprising a mutant ITR, which has a deleted B region. The library is integrated such that it is proximal to the ITR (e.g., the wild-type ITR or mutant ITR). In a subsequent cloning step, each promoter library (e.g., comprising the wild-type ITR or mutant ITR) is barcoded with a 20 nt degenerate base pair nucleotide tag. At this point, each promoter::barcode library is sequenced using an appropriate HTS sequencing machine to determine the promoter and barcode sequences and their association. In a subsequent cloning step, the screening cassette, consisting of the CMV minimal promoter and the GFP, reporter is inserted into the promoter constructs. This cloning step integrated the barcode into the transcribed portion and is therefore used as a marker of gene expression and thereby promoter strength.

Amplicons are generated to determine the input and output frequency of barcodes, which are associated with the synthetic promoter population. The input barcode frequency data is generated prior to transfection into CHO-S using the library DNA as template. Post transfection RNA is extracted from cells and the synthesized cDNA is used to generate output amplicons. Illumina tags and indeces, which are part of the amplicon primers, allowed for direct sequencing of the amplicon population therefore generating unskewed quantitative data to determine barcode frequencies. Both amplicon populations are sequenced (e.g., tag-sequencing) (HiSeq) and data readings are normalized using the input over output barcode frequency. Bioinformatic analysis and integration of the various sequencing datasets identify functionally active synthetic promoters.

Bioinformatic analysis is performed to identify the PWM building blocks, used for construction of each synthetic promoter library. The RNA sequencing data generated is used to identify high expressing genes and transcription factors, which found 144 and 48 respectively. The promoter region of the highly expressed genes (−250 to +50 relative to TSS) is subjected to an overrepresentation analysis to isolate positional weight matrixes (PWMs). A pool of 146 enriched PWMs is identified in the set of 144 promoters when compared to the CHO promoterome. A subsequent association analysis found that 13 PWMs are binding sites of the set of 48 highly expressed TFs. The 13 PWMs are used to construct a new SP library termed HK4 (FIG. 15).

The library cloning strategy is outlined herein in FIG. 15. Briefly, the identified PWMs are synthesized and the DNA string is digested using specific compatible restriction enzymes to liberate the individual building blocks. The next step includes the re-ligation to associate the PWMs in a shuffled fashion. The protocol allows for the PWMs to be associated in either orientation and any combination, generating a high complexity library using a relatively small number of PWMs. In a final step PCR is performed to add homology arms to the individual library constructs, which enabled the integration into the screening vector using an efficient recombination approach. This library cloning approach delivers synthetic promoter candidates ranging from 150 bp to 600 bp with a total library complexity of 1.2×10⁶ unique constructs.

To validate the bioinformatics approach for the identification of the PWMs, each library is transfected into CHO-S cells using a lipid base approach. In two individual experiments, two carrier DNA vectors are co-transfected with each library. Carrier DNA is used to decrease the number of library constructs in the transfection whilst keeping the total DNA amount used for transfection constant. This is done to avoid transfection of a single cell with multiple library constructs, which may lead to promoter cross-talk and thus distort GFP output readings. As the two carrier DNA vectors differed in size (e.g., kpMK-RQ: smaller than library constructs and pShuttle: larger than library constructs), different transfection ratios of 1:100 and 1:1000 respectively are used due to a more efficient plasmid uptake of smaller vectors. FACS analysis of each CHO-S cell population transfected with either promoter library (e.g., transfected with the library comprising a wild-type ITR, or the library comprising the mutant ITR) is performed to determine the number of GFP positive cells and the mean GFP intensity. Co-transfecting the carrier vectors with each library showed that both, the number of GFP positive cells and the mean GFP intensity is increased for the HK4 promoter library when compared to the background (FIG. 16). Previous shuffled promoter libraries showed a discovery rate of 0.5% to 2% of functional promoters within a library. The increase in GFP intensity, which can solely be contributed to the functional library population (0.5% to 2%), validates the bioinformatics analysis for the identification of PWMs contributing to constitutive promoter activity. It further demonstrates that the PWMs are combined to high activity promoters.

To screen each synthetic promoter library with NGS, a cloning protocol is devised which aligns with the sequencing requirements for (I) library::barcode association and (II) barcode sequencing (FIG. 2). The library population is size selected to comply with a sequencing length restriction of 300 bp reads paired end. To this end the 200 bp to 400 bp library fraction are selected and size separated. This library fraction is cloned into a screening vector containing a poly-linker and the SV40 polyA site, and is found to have a complexity of approximately 70,000 unique constructs. In a subsequent step, the 20 nt degenerate barcode is inserted with a 4-fold coverage of each library. This promoter::barcode population is sequenced using MiSeq to determine the promoter and barcode sequences and their association. Subsequently, a CMV minimal promoter::GFP screening cassette is inserted downstream of the synthetic library element, upstream of the barcode with a 5 fold coverage. This final cloning step transferred the barcode into the 3′ portion of the transcribed DNA making it possible to use the barcode frequency as read out of promoter activity. Stringed cloning quality control steps are implemented to ensure a close to 100% cloning success rate at every step.

A CHO-S population of fife flasks with 10e7 cells are transfected with either of the promoter libraries (e.g., transfected with the library comprising a wild-type ITR, or the library comprising the mutant ITR) Several standard promoters (e.g., CMV-IE, CMV minimal promoter, EF1a, PGK and the empty GFP vector) are co-transfected with the library at 0.10% of the library (0.02% of each control). Each standard promoter is previously barcoded with 7 different barcodes. Samples are taken 24 hours (5×) and 48 hours (4×) post transfection (pt) and total RNA extracted for cDNA synthesis. Subsequently DNA amplicons is generated using qPCR and specific primers incorporating the Illumina barcodes and adapters to enable direct sequencing. Amplicon generation is done for the DNA input sample and the nine output samples.

Bioinformatics Analysis of MiSeq Data: Promoter Barcode Association Sequencing

The sequencing to associate promoters with barcodes is performed via a paired end MiSeq approach. MiSeq allows a total sequencing length of 300 nt, enabling the paired end sequencing of DNA of up to 500 nt. Sequence analysis determines a total complexity of 276 thousand promoters and approximately 1 million unique barcodes are identified. This is consistent with the estimated 4-fold promoter barcode coverage.

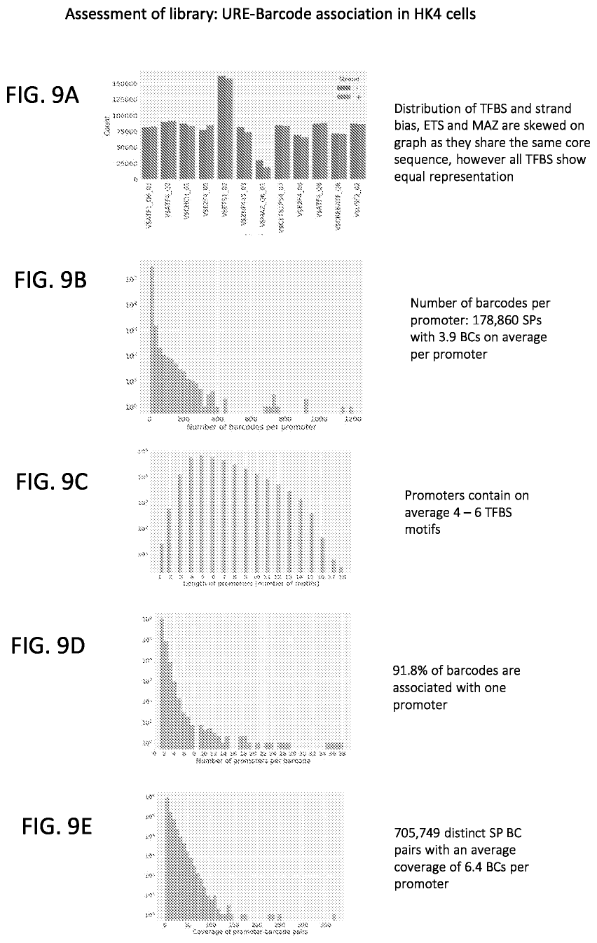

Further barcode analysis of a library expressing the wild-type ITR, found that 95% of all barcodes (994 thousand) are associated with one promoter and only 5% of the barcodes are associated with more than one promoter. Promoters from HCS are identified based on low variance among the barcodes of the same promoter, therefore promoter barcode association is analyzed. Only approximately one third of the library (32%: 89 thousand promoters) are associated with only one barcode. In contrast 68% (187 thousand promoters) showed association with multiple barcodes. A PWM analysis showed that the majority of promoters combined a number of 4 to 6 motifs. The maximum PWM number is found to be 18 whereas a considerable number of promoters showed a PWM number of 1 to 3 (FIG. 9C). We found in previous experiments that a number of at least 5 PWM is required to drive high expression. Overall distribution of the PWM showed that motifs are equally distributed within the promoters and show no strand bias. This analysis however is skewed by two PWM that share the same core sequence (ETS1 and MAZ) and therefore appeared unevenly represented (FIG. 9A). Manual validation of ETS1 and MAZ integration confirmed equal distribution of both PWMs. Furthermore, no strand bias of PWM integration is found (FIG. 9A). Similar results are found in via further barcode analysis of a library expressing the mutant ITR (data not shown).

Bioinformatics Analysis of HiSeq Data: Barcode Quantification Sequencing

To validate the data generated by HiSeq of the 24 h pt and 48 h pt amplicons, the expression strength of the included standard promoters (e.g., CMV-IE, CMV minimal promoter, EF1a, PGK and the empty GFP vector) is determined. Activity of the standard promoters driving the eGFP reporter is very tight, with low variance between the 7 barcodes. Activity is also reproducible across different samples taken on the same day, and there is a good correlation of the 24 h sample with the 48 h sample (FIG. 17).

Analysis of the entire HiSeq data set found a total number of 6 million barcodes. This exceeds the number of barcodes identified in the promoter barcode association by 5 million. Within the identified barcodes, 729 thousand are previously found in the promoter barcode association sequencing. Encouragingly, the set of 729 thousand barcodes corresponds to 91% of the promoters (252 thousand) present in the promoter barcode association sequencing data set. Thus, the barcode quantification sequencing captures the majority of the barcodes whereas the sequencing depth of the promoter barcode association sequencing may present a bottleneck to capture the entire barcode pool.

Validation of Candidate Promoters

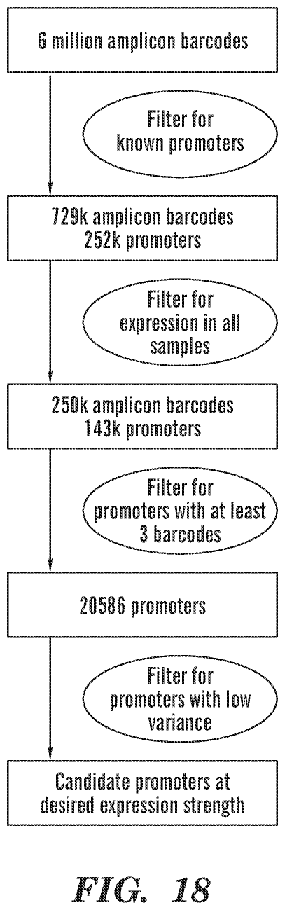

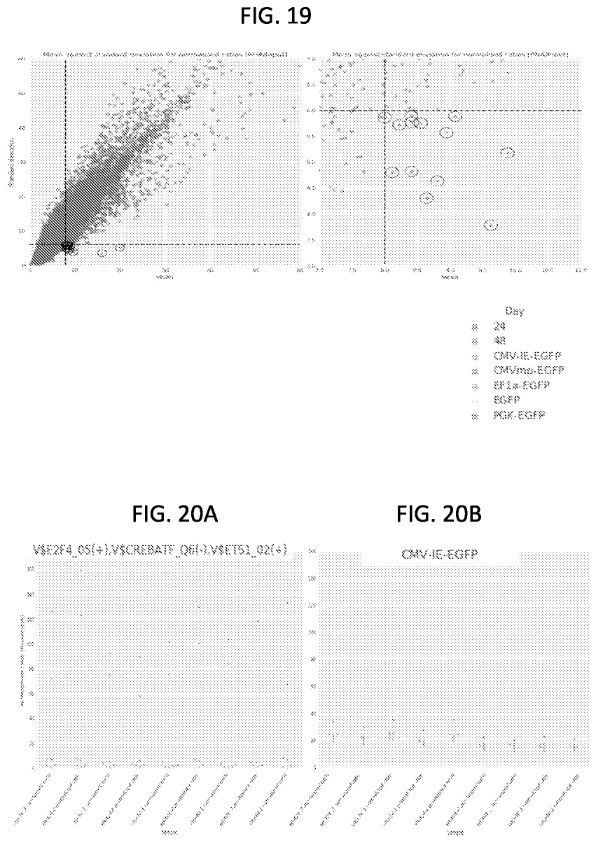

To select candidate promoters for validation of the HCS methodology, a workflow with specific criteria is applied to each population (FIG. 18). Importantly only promoters which are associated with at least three different barcodes and represented in all 10 samples (DNA input and nine amplicon output samples from 24 h pt and 48 h pt) are included in the final analysis. As there is a slight shift in expression level of the standard promoters in the 24 h pt compared to the 48 h pt output samples (FIG. 17), the barcode frequency of the two output sample time points are not combined but treated separately. This approach delivered 20586 promoters from a population expressing a wild-type ITR, which subsequently are filtered for low variance (standard deviation below 6) among the individual barcodes (FIG. 19). These promoters are compared to the promoters identified from the population expressing a mutant ITR. If a promoter is identified as being active in the presence of a wild-type ITR, but not identified as being active in the presence of a mutant ITR, this indicates that the promoter activity is dependent on the overall 3D conformation of the vector. Only promoters with activity dependent on the 3D conformation of the vector

Initially a small set of promoters with activity dependent on the 3D conformation of the vector, and showing half to equal expression strength of the CMV-IE standard promoter are selected for validation (FIG. 19). This set includes candidates from time point's 24 h pt and 48 h pt. It is worth mentioning however that an increased variation is observed among the barcodes when comparing synthetic promoter candidates to standard promoters. FIGS. 20A and 20B shows the variation of 7 different barcodes when associated with a synthetic- and the CMV-IE promoter.

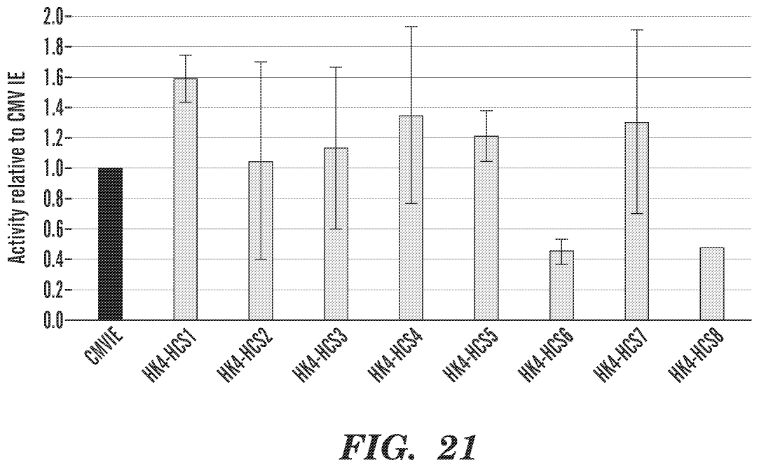

Eight synthetic promoters are synthesized for validation driving the firefly luciferase reporter (FIG. 21). Plasmids are transfected into CHO-S and reporter assays are done 24 hours after transfection. The luciferase reporter readout shows that all promoters are functional. Whilst the identified promoters show an overall higher expression level than expected, the activity remains within acceptable variance range. It is also important to note that the validation of the candidate promoters used reporter protein readout whereas identification of the candidates is based on the transcript level. The difference between the protein and messenger RNA level including mRNA stability and translation efficiency may account for the observed difference in activity readout.

### Materials and Methods

CHO-S Maintenance and Transfections

FreeStyle™ CHO-S cells (Invitrogen, R800-07) are grown in FreeStyle™ CHO Expression medium (Gibco, 12651014) supplemented with 8 mM GlutaMAX™ (Gibco, 35050061). Cells are grown in shaker culture in either 250 ml flasks (Corning, 431144) or 500 ml flasks (Corning, 431145), using the following conditions: 37° C., 8% CO₂, 75% relative humidity, 120 rpm, 25 mm throw (Infors Minitron). Cells are passaged every 3 to 4 days, i.e. twice per week, to a cell density of 3×10⁵ cells/ml.

Cells are passaged at a cell density of 6×10⁵ cells/ml the day before transfection. On the day of the transfection, cells are counted using a disposable hemocytometer (NanoEnTek, DHC-N01). A cell density of 10⁶ cells/ml is required for transfection. Cells are diluted in pre-warmed medium if cell density is above 10⁶ cells/ml. 10 ml cells at 10⁶ cells/ml (10⁷ cells) are transferred into 125 ml flasks (Corning, 431143). Transfections are performed using FreeStyle MAX Reagent (Invitrogen, 16447-100). For each transfection, 200 μl OptiPRO SFM (Gibco, 12309019) is added to 10 μg DNA and mixed by pipetting. 55 μl FreeStyle MAX Reagent is added to 1.1 ml OptiPRO SFM and mixed by pipetting. 210 μl FreeStyle MAX Reagent mix is added to each DNA mix, mixed by pipetting, and incubated at room temperature for 20 minutes. 40 μl transfection mix is added dropwise to 10 ml cells. Library is transfected in five replicates.

Sampling

Samples are collected 24 hours and 48 hours post transfection. Samples from all five flasks are collected at 24 hours, and samples from four flasks are collected at 48 hours. 3 ml cells are collected and pelleted at 100 g for 3 mins. Supernatant is removed using a VacuSafe (Integra, 158320), 350 μl buffer RLT (Qiagen, 79216) with 1% β-mercaptoethanol (Sigma-Aldrich, M6250) is added and cell pellet is lysed by vortexing.

RNA Extraction, DNase Treatment and cDNA Synthesis

RNA is extracted using RNeasy mini kit (Qiagen, 74104) according to manufacturer's instructions. RNA is eluted in 50 μl nuclease-free water. RNA is quantified using Qubit™ RNA BR Assay Kit (Invitrogen, Q10210) with a Qubit 3.0 fluorimeter (Invitrogen, Q33216). 10 μg RNA is used for DNase treatment with DNA-free™ DNA Removal Kit (Invitrogen, AM1906) according to manufacturer's instructions. 300 ng DNase-treated RNA is used for cDNA synthesis with SuperScript™ III Reverse Transcriptase (Invitrogen, 18080044) with addition of RNaseOUT™ (Invitrogen, 10777019) and using oligo(dT) primers (Invitrogen, AM5730G), according to manufacturer's instructions.

Amplicon Generation

Amplicons are generated using qPCR, with four replicates for each cDNA sample and the input sample. RNA and a no template control are included as controls, with one replicate each. Each of the nine sample is amplified using a different barcoded forward primer (Table 1). The same reverse primer is used for all reactions including the input.

TABLE 1

| | | SEQ |
|---|---|---|
| | | ID |
| ID | Sequence 5-3 | NO: |
| LEFTbc01 | CAAGCAGAAGACGGCATACGAGATACGAGACTGATTA | 18 |
| | GTCAGTCAGCCCAAAGACCCCAACGAGAAGC | |
| LEFTbc02 | CAAGCAGAAGACGGCATACGAGATGCTGTACGGATTA | 19 |
| | GTCAGTCAGCCCAAAGACCCCAACGAGAAGC | |
| LEFTbc03 | CAAGCAGAAGACGGCATACGAGAT | 20 |
| | ATCACCAGGTGTAGTCAGTCAGCCCAAAGACCCCAAC | |
| | GAGAAGC | |
| LEFTbc04 | CAAGCAGAAGACGGCATACGAGATTGGTCAACGATAA | 21 |
| | TGCAGTCAGCCCAAAGACCCCAACGAGAAGC | |
| LEFTbc05 | CAAGCAGAAGACGGCATACGAGATATCGCACAGTAAA | 22 |
| | GTCAGTCAGCCCAAAGACCCCAACGAGAAGC | |
| LEFTbc06 | CAAGCAGAAGACGGCATACGAGATGTCGTGTAGCCTA | 23 |
| | GTCAGTCAGCCCAAAGACCCCAACGAGAAGC | |
| LEFTbc07 | CAAGCAGAAGACGGCATACGAGATAGCGGAGGTTAGA | 24 |
| | GTCAGTCAGCCCAAAGACCCCAACGAGAAGC | |
| LEFTbc08 | CAAGCAGAAGACGGCATACGAGATATCCTTTGGTTCA | 25 |
| | GTCAGTCAGCCCAAAGACCCCAACGAGAAGC | |
| LEFTbc09 | CAAGCAGAAGACGGCATACGAGATTACAGCGCATACA | 26 |
| | GTCAGTCAGCCCAAAGACCCCAACGAGAAGC | |
| LEFTbc10 | CAAGCAGAAGACGGCATACGAGATACCGGTATGTACA | 27 |
| | GTCAGTCAGCCCAAAGACCCCAACGAGAAGC | |
| RIGHThcs | AATGATACGGCGACCACCGAGATCTACACTATGGTAA | 28 |
| | TTGTGCCCCGACTCTAGGAATTCA | |

qPCR is performed on a Rotor-Gene Q 5plex HIRM Platform (Qiagen, 9001580) in a 72-well rotor. A reaction volume of 20 μl is used, containing the following reagents: 10 μl 2× QuantiNova SYBR Green PCR Master Mix (Qiagen, 208056), 0.4 μl forward primer (10 μM), 0.4 μl reverse primer (10 μM), 7.2 μl nuclease-free water, 2 μl template. cDNA is used undiluted, whereas the input DNA sample is diluted 1:5000. The following PCR program is used: 95° C. for 2 min, then 25 cycles of 95° C. for 5 sec, 60° C. for 10 for cDNA samples, and the same program but with 29 cycles for the DNA input sample.

The four replicates of each cDNA sample and the four replicates of the DNA input sample are combined, and each pool is purified using Agencourt AMPure XP beads (Beckman Coulter, 10136224) according to manufacturer's instructions, using a 1:1 ratio. DNA concentrations are measured using Qubit™ dsDNA BR Assay Kit (Invitrogen, Q32850) with a Qubit 3.0 fluorimeter. The purified samples are further combined into two pools, one with the five samples taken at 24 hours, and one with the four samples taken at 48 hours and the DNA input sample, using equimolar amounts of each sample. Both pools are again purified with Agencourt AMPure XP beads, using a 1:1 ratio. The two pools are submitted for NGS.

### Example 2

Identification of Unique Regulatory Elements from AAV Libraries

Synthetic promoter libraries for identifying unique regulatory elements are described herein above in Example 1. To identify unique regulatory elements in an AAV, promoter libraries are used to generate an AAV library. AAV libraries are generated in HEK 293T cells using the calcium phosphate transfection method. Briefly, 25 T225 flasks are seeded with 8E⁰⁶ cells per flask in 40 ml media two days prior to transfection. On the day of transfection cells are between 80% and 90% confluent. 20 ml of media per flask is replaced with fresh media 1.5 hrs prior to transfection and a mixture of 40 ug pAd5 helper plasmid and 2 ug library plasmid in 4 ml 300 mM CaCl2) per T225 is prepared. Equal amounts of CaCl2)/DNA mix and 2×HBS (280 mM NaCl, 50 mM HEPES pH 7.28, 1.5 mM Na2HPO4, pH 7.12) are mixed and 8 ml of the mixture is added to each flask. After 3 days cells are detached with 0.5 ml 500 mM EDTA each flask and the cell pellet is resuspended in Benzonase digestion buffer (2 mM MgCl2, 50 mM Tris-HCl, pH 8.5). AAVs are released from the cells by submitting them to three freeze-thaw cycles, non-encapsidated DNA is removed by digestion with Benzonase (200 U/ml, 1 hr 37° C.), cell debris is pelleted by centrifugation, followed by another CaCl2) precipitation step (25 mM final concentration, 1 hr on ice) of the supernatant and an AAV precipitation step using a final concentration of 8% PEG-8000 and 625 mM NaCl. Virus is resuspended in HEPES-EDTA buffer (50 mM HEPES pH 7.28, 150 mM NaCl, 25 mM EDTA) and mixed with CsCl to a final refractory index (RI) of 1.371 followed by centrifugation for 23 hrs at 45000 Rpm in a ultracentrifuge. Fractions are collected after piercing the bottom of the centrifuge tube with a 18 gauge needle and fractions ranging in RI from 1.3766 to 1.3711 are pooled and adjusted to an RI of 1.3710 with HEPES-EDTA resuspension buffer. A second CsCl gradient centrifugation step is carried out for at least 8 hrs at 65000 Rpm. Fractions are collected and fractions with an RI of 1.3766 to 1.3711 are dialyzed overnight against PBS, followed by another 4 hr dialysis against fresh PBS and a 2 hr dialysis against 5% sorbitol in PBS. All dialysis steps are carried out at 4° C. Virus is recovered from the dialysis cassette and pluronic F-68 is added to a final concentration of 0.001%. Virus is sterile-filtered, aliquoted, and stored in aliquots at −80° C. Genomic DNA is extracted from 10 ul of the purified virus using the MinElute Virus Spin Kit (Qiagen Cat #57704), and the viral genome titer is determined by qPCR using an AAV2 rep gene specific primer probe set (repF: TTC GAT CAA CTA CGC AGA CAG, (SEQ ID NO: 11); repR: GTC CGT GAG TGA AGC AGA TAT T (SEQ ID NO: 12), rep probe: TCT GAT GCT GTT TCC CTG CAG ACA (SEQ ID NO: 13)).

In order to measure the strength of a URE of the AAV library in vitro, the AAV library is expressed in a hepatocyte. mRNA is extracted from hepatocytes expressing the AAV library using an mRNA extraction kit obtained from ThermoFisher (catalog number 61006). The protocol for mRNA extraction provided with the kit is followed. mRNA is purified and used as a template to synthesize cDNA using ProtoScript® First Strand cDNA Synthesis Kit obtained from New England Biolabs (catalog number E6300S). The protocol for cDNA synthesis provided with the kit is followed.

In order to measure the strength of a URE of the AAV library in vivo, the AAV library is administered to a mouse via tail vein injection. To stimulate dilation of the tail vein prior to injection, mice are placed in a warm incubator (e.g. at 28-30° C.) for up to 30 minutes. 4 days post injection, injected mice are euthanized and their livers are removed via standard surgical procedures. RNA is extracted from the whole liver tissue using an RNA extraction kit obtained from ThermoFisher (e.g., catalog number AM7960). The extracted RNA is purified and used as a template to synthesize cDNA using ProtoScript® First Strand cDNA Synthesis Kit obtained from New England Biolabs (catalog number E6300S). The protocol for cDNA synthesis provided with the kit is followed

For both in vivo and in vitro methods, barcode sequence is amplified from the cDNA using primers that include index primers and P7 and P5 oligos for direct Illumina sequencing. The left primer (leftBC) has a sequence of CAAGCAGAAGACGGCATACGAGATACGAGACTGATTAGTCAGTCAGCCCTCCG CCTTGCCCTGA (SEQ ID NO: 14), and the right primer (Right_UPAS) has a sequence of AATGATACGGCGACCACCGAGATCTACACTATGGTAATTGTTCCTACTATTCCG TACCGTAGGGT (SEQ ID NO: 15). Sequencing is used to measure the content of each of the plurality of barcodes present in a given amplicon. This amplified content of each of the barcode is the barcode output. The barcode output is normalized to the barcode input, which is the content of each unique barcode. The normalized ratio is the expression frequency, and is an indicator of the strength of the URE associated with the barcode in relation to the ITR (e.g., the wild-type ITR or mutant ITR). For example, having a higher expression frequency of a barcode in the backbone having a wild-type ITR as compared to the backbone having a mutant ITR indicates that that function of the URE is regulated by the ITR, e.g., the B region of the ITR.

### Example 3

AAV Sequencing Using PacBio

The High content screening (HCS) analysis method used for the identification of barcode frequencies within a multiplexed pool of regulatory elements (see herein above in Example 2) relies on the comparison of input and output data. Both data sets are generated using NGS sequencing. The proof of concept of the HCS analysis i done using an in vitro cell line where input and output data can be generated using the plasmid DNA used for transfection and amplicons generated from the cDNA of transfected cells. The ratio of constructs is assumed to stay constant between the plasmid DNA used for transfection and the transfected DNA within the in vitro cell line, however, this varies to the in vivo system. It is generally assumed that the ratio of different multiplexed constructs present in a plasmid DNA prep will be altered during AAV production and packaging of the episomes. The construct ratio will be further distorted through the injection process where only a subpopulation of injected AAV particles will be retained within the target tissue. It is therefore of advantage to assess the constructs present in the AAV prep. The technology chosen to sequence the AAV episomes is PacBio which relies on the ligation of the bell adaptor to double stranded DNA.

Single stranded copies of the AAV episome will be packaged during generation of the AAV prep where either the plus or minus strand can be present. As the PacBIo sequencing technology relies on double stranded DNA, a method was established that allowed the isolation of episomes from the AAV capsid and episome second strand synthesis for sequencing. This method is of particular relevance as single stranded episomes have a tendency to form double stranded duplexes when isolated. However, as each AAV episome carries a unique barcode, two single stranded episomes will create a mixed barcode duplex. The established method circumvents this hurdle and allows the sequencing of the packaged AAV episomes.

Experimental Procedure:

100 μL of AAV suspension was divided into 3*32 μL aliquots, each in a 1.5 mL microcentrifuge tube. These were handled identically and in parallel. To each 32 μL aliquot was added: 5 μL DNAse I Buffer (NEB B0303S), IOU DNAse I (Life Technologies 90083), and PBS to reach a final volume of 50 μL. Tubes were then incubated for 30 min at 37° C. to degrade free DNA in the virus prep. 150 μL of sterile PBS was added to each tube, after which the resulting 200 μL mixtures were subjected to protease K digestion, cleanup, and elution of purified viral DNA, all using the High Pure Viral DNA extraction Kit (Roche 11858874001) according to manufacturer's instructions. The resulting triplicate 50 μL tubes of purified virus genomes were used for subsequent second strand synthesis.

Random hexanucleotides were added to each sample and heated for 5 min at 95° C. and immediately placed on ice. Subsequently the polymerase was added to the AAV genomes and placed into a precooled thermocycler. Hybridization of random hexamers was done by a gradual temperature increase from 4° C. to 37° C. with 0.1° C./sec increments followed by DNA polymerization at 37 C for one hour. The reaction was stopped with the addition of 0.5M EDTA. Next 300 μL of dH2O and 100 μL protein precipitation solution was added and vortexed for 20 sec at high speed. The mixture was incubated for 5 min on ice and centrifugated at 16,000 g at 4° C. The supernatant was mixed ten times with 300 μL isopropanol and 2 μL glycogen by inversion. The second strand synthesis reaction was incubated at 20 C for 12 hours and centrifugated at 25,000 g for 45 min at 4° C. Next the reaction was cooled on ice for 5 min before the supernatant was carefully discarded. The pellet was washed with 300 μL of 70% ethanol and centrifugated for 10 min at 25,000 g at 4° C. The supernatant was carefully discarded and the pellet air-dried for approximately 1 hour before resuspending in 30 μL 5 mM Thris-HCl pH 8.5. An appropriate amount was used for ligation of PacBio adapters according to the manufacturer's instructions.

Results:

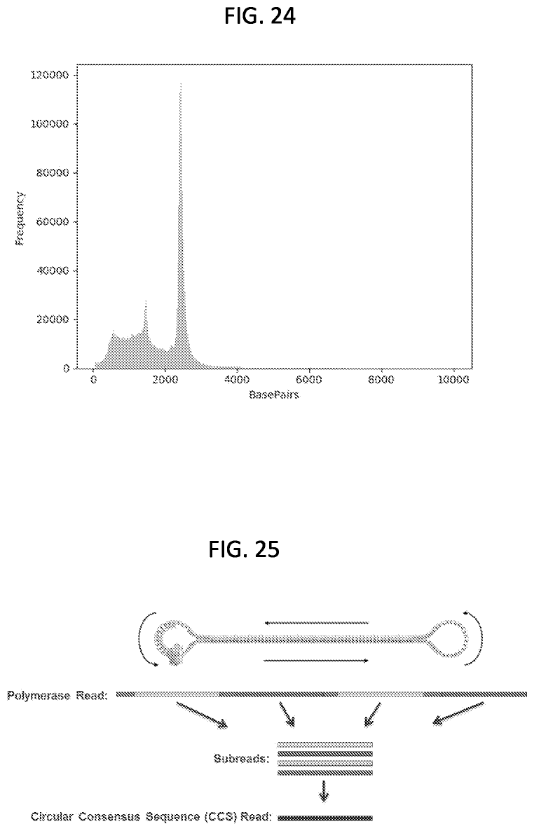

AAV genomes which have been subjected to the second strand synthesis protocol (described above) were submitted for PacBio library preparation and sequences on the PacBio Sequel platform by Edinburgh Genomics. This produced ˜9M reads with a median length of ˜2200 bp (FIG. 24).

The size distribution of the reads is shown in FIG. 24, the large peak at ˜2500 bp fits with the expected size of the AAV genome including ITRs. 49% of reads fall into the 2000-3000 bp size range. It is possible that shorter sequences are truncated AAV genomes or pairs of single stranded, partially complementary AAV genomes that have formed duplexes.

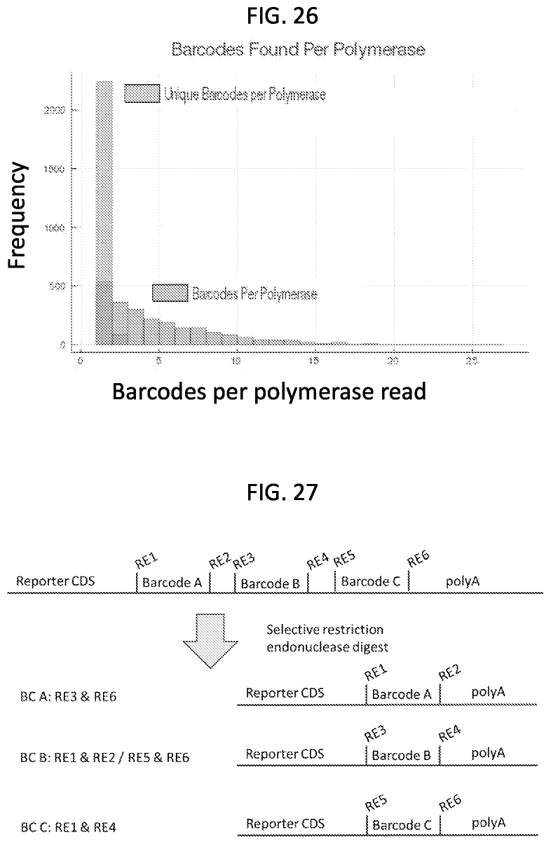

PacBio reads are made up of Polymerase reads and Subreads (FIG. 25). If a molecule is derived from chimeric sequence it is likely that it will have 2 unique library barcodes per polymerase read. In order to address the scenario in which the second strand synthesis and end repair may have generated chimeric reads; reads were grouped by polymerase ID, library barcodes (from a whitelist of 12,000 possible library barcodes) were searched for (FIG. 26).

The majority of polymerase IDs have only one library barcode, with a very minor proportion of Polymerase families having more than two. Zero polymerase reads have more than two identifiable library barcodes.

### Example 4

Cloning of Small and High Complexity Library into AAV Vector

The successful cloning of a multiplexed library depends on an efficient cloning procedure to retain the library complexity. This is of particular importance in the case of the high complexity, 12000 construct library. The cloning of the library is a stepwise process starting from construct synthesis to final transfer into the AAV vector backbone where each step has the potential to skew the construct ratio. Thus it is important that a cloning redundancy of construct number is applied at each step to ensure that all constructs are being carried over and the complexity of the library is retained. Redundancies when libraries are cloned are usually between a minimum of 3 to 5 fold of constructs for each cloning step. A library size of 12,000 constructs that relies on 3 cloning steps requires therefore a minimum of roughly 350,000 cfu's when transferred into the AAV vector. A cloning procedure was optimized in order to allow for successful and efficient transfer of the library into the AAV vector, which would guarantee that construct numbers are retained. This method takes the low copy origin of replication of the AAV vector into account and is compatible with growing conditions, such as lower temperature and reduced shaking speed, to maintain the integrity of the AAV ITRs.

Experimental Procedures

The 12,000 construct and the 80 construct library were both cloned using the same method (see herein above in Example 2). Two μg of each, the library and the self-complementary AAV vector (SCAAV3) were digested with the restriction endonucleases SgrAI (New England Biolabs) and PacI (New England Biolabs) for 3 h at 37° C. Next the linearized SCAAV3 vector and the library fragments were isolated and purified by agarose gel electrophoresis (1% gel). The library was then ligated into the SCAAV3 vector backbone using T4 ligase (New England Biolabs) and incubated for 1.5 hours at 21° C. followed by heat inactivation for 10 min at 65° C. Subsequently electrocompetent Endura e-coli cells (Lucigen) were used to transform 1 μl of the library ligation into 25 μl cells according to the manufacturer's instructions. To assess transformation efficiency 1 μl and 10 μl pf the transformation was plated onto LB-agar with kanamycin and incubated at 32° C. Glycerol was added to the remaining transformation mix in a 1:1 ratio, which was then stored at −80° C. After establishing that the transformation efficiency was high enough to account for all constructs, a sufficient amount of glycerol stocks were defrosted and cultured for Zymogen endotoxin free giga preps, which were performed according to the manufacturer's instructions. ITR integrity was verified by restriction endonuclease digestion with SmaI and where necessary the DNA was precipitated in order to increase the concentration. To each sample 1/10 volume of 3M sodium acetate pH 5.2 and 2.5 volumes 100% ethanol was added. This was mixed by inverting and incubated for 1 hour at −20° C., followed by centrifuging 1 hour at 4800 g. The supernatant was removed and the pellet was washed twice with 500 μl 70% ethanol. The pellets were air dried and resuspended in an appropriate volume of TE pH 8.

### Example 5

Generation of Multiple Barcoded Constructs for NGS Screening

The HCS readout relies on quantitative normalized barcode readings that can be directly correlated to the activity of a given regulatory element. During the cloning and screening process, experimental biases can alter the barcode quantification leading to “false” positive or skewed readouts. Multiple barcodes at the 3′ end of the reporter CDS for the same regulatory element circumvent this and provide statistical credibility to the collected data.

Depending on library size it can be costly and time consuming to synthesize each regulatory element in a multiplexed library with three distinct barcodes. We utilized a method where three barcodes are synthesized simultaneously and are flanked with compatible type II restriction endonuclease recognition sites. This allowed the generation of individually barcoded regulatory elements through restriction digest and self-ligation. Initially the constructs within the library were pooled in an equimolar ratio and then divided into three separate pools. Each pool was then subjected to a different restriction endonuclease digestion with compatible enzymes to selectively delete two of the tree barcodes. This method allows the generation of multiple barcodes for the same construct thereby aiding statistical analysis of the collected NGS data.

Experimental Procedure

Constructs were pooled in an equimolar ratio and divided into three sub pools. Selective restriction endonuclease digestion of 2 μg DNA of each pool was performed according to the manufacturers specifications (FIG. 27). Resulting linearized plasmid DNA was ligated using T4 ligase according to the manufacturers specifications for (2 hours). Subsequently, 2 μl were transformed into ecoli (NEB10β) cells and grown overnight in a liquid culture at 37° C. Simultaneously, some transformation mix was cultured on agar plates in order to determine the transformation efficiency so that all the constructs would be accounted for. Separate colonies were picked and grown up for Qiagen Mini Preps and the barcodes in the plasmids were sequenced. There turned out to be a good variation of barcodes, and in none of the sequenced clones more than one barcode was present. Plasmid DNA was then extracted from the liquid cultures using a Qiagen Midi Prep kit according to the manufacturer's instructions.

### Example 6

Tissue and Downstream Processing for NGS Analysis

Determining the CNS specificity of the library relies on successful determination of barcode frequencies in the target and non-target murine tissues. The HCS procedure uses NGS data which is generated through amplicon sequencing of the in-put and output consisting of AAV genomes and RNA/cDNA respectively.

The harvested murine tissues include elastic (muscle, heart, aorta, diaphragm) and soft (liver, spleen and brain) tissues. Tissue architecture determines the way in which the tissue is processed using a Beadbug homogenizer in combination with an Allprep nucleic acid extraction kit. The latter makes it possible to extract both DNA and RNA simultaneously thus allowing the generation of input (AAV genome) and output (RNA/cDNA) amplicons for NGS determination of barcode frequencies. Depending on tissue type, zirconium spheres of different weights in combination with garnet shards are used for tissue homogenization.

Brain tissue was extracted as follows. An appropriate volume of Allprep reagent RLT plus buffer was prepared by the addition of B-mercaptoethanol according to the manufacturers description and an appropriate volume depending on weight of harvested brain tissue transferred into Beadbug tubes containing 6 mm zirconium spheres. Next the brain sample (max weight 30 mg) was homogenized for 2×0.5 minutes at 350 rpm, incubated on ice for 10 min and centrifuged according to manufacturer's instructions. Then 350 μl homogenate from each sample was transferred to a Allprep column and a second portion to a new 1.5 ml Eppendorf tube and fast frozen with EtOH and dry ice before transferring it to a −80° C. freezer. RNA and DNA was subsequently isolated according to the manufacturer's instructions where RNA extraction was done first followed by DNA extraction. RNA was eluted in 50 μl RNase free water and DNA in 100 μl EB buffer. Extracted brain RNA and DNA was stored at −80° C. and −20° C. respectively. The concentration of the RNA samples was determined and treated with rDNase I (2U) according to the manufacturer's instructions and the concentration was re-quantified.

cDNA synthesis and incorporation of unique molecular identifiers (UMIs) using the brain RNA were performed as followed. UMI incorporation was done to account for PCR stochasticity during amplicon preparation. The UMIs can be used to keep track of how many cycles of PCR a molecule has gone through.

This extra step in the adapter ligation process was tested using a low complexity library which contains 10 barcoded CMV-ie constructs. This process was carried out for 24 technical replicates (PCR duplicates in this case). CMV-ie barcode counts were compared between all technical replicates and Pearson correlation calculated to assess reproducibility.

cDNA synthesis using Superscript III was done with a gene specific cDNA primer incorporating the 18 nucleotides (nt) long UMI according to manufacturer's instructions. Samples were incubated at 65° C. for 5 min then at 4° C. for 1 min in thermal cycler. Synthesis was done for both a cDNA and reverse transcriptase negative reactions. The thermal cycler was preheated to 55° C. Samples were loaded into the thermal cycler at 55° C. and run for 50 min; then the enzyme was inactivated at 85° C. for 5 min.

DNA from the homogenised tissue was extracted to isolate the AAV genomes for the generation of input NGS data. This was done in a subsequent step after tissue homogenisation using the Allprep sample kit according to the manufacturer's instructions.

For subsequent amplicon generation of both, the input and output samples using DNA/AAV genomes and cDNA respectively, a QPCR reverse primer is used homologous to the downstream region of the incorporated UMI. This primer annealing site was incorporated during cDNA first strand synthesis as described above. For amplicon generation using QPCR, 4 μl containing 2 ng of template was used within a reaction 20 μl including 2× QuantiNova mastermix, carboxyrhodamine, forward and reverse primers and nuclease free water at appropriate concentrations. A similar reaction was set up with a house keeping primer set to monitor and assess the efficiency of cDNA synthesis. Also included in the QPCR reactions are standards at various dilutions to control for the efficiency of the QPCR amplification reaction.

To assess specific amplification, the generated QPCR amplicon is subjected to agarose gel electrophoresis, excised and purified from the agarose gel using Quiagen gel extraction according to manufacturer's instructions and Sanger sequenced. Next an additional amplicon test QPCR run is performed to determination of the concentration of generated amplicons and the QPCR cycle number. Generated amplicons are harvested within the first quarter of the QPCR run within the linear amplification range. This is of particular importance to avoid over amplification and the introduction of specific biases within the amplicon pool.

Forward and reverse primers used for the amplicon generation incorporate Illumina P7 and P5 oligo, Read 1 and Read 2 primer site and i7 index. The use of these elements in combination with the specific primer sequence makes it possible to directly sequence generated amplicons without an additional step incorporating the multiplexing index. For different amplicon populations different i7 index sequences are being incorporated allowing the differentiation of sequencing samples. Furthermore, primers are synthesized with a 3′PS bond modification that allows the binding to the SP sequencing flow cell and enables direct amplicon data generation. This method is applied for the collection of barcode frequency data from input (AAV genomes) as well as output (cDNA) material from a variety of different tissues including brain, skeletal and smooth muscle, liver and spleen.

### Example 7

### 1. Selection of Genes Upregulated in Colorectal Cancer

Genes are identified by a meta-analysis of microarray data from colon cancer sources from a study conducted by Rhodes et al (Rhodes et al (2004) PNAS 2004; 101; 9309-14). This resulted in the identification of the 17 genes (data not shown) shown to be upregulated in colorectal cancer biopsies.

These 17 genes are then screened to ensure that overexpression is a result of altered transcription factor activation, instead of chromosomal amplification, in order to select cis-regulatory elements that will be active in the context of an altered transcription factor environment. This resulted in the exclusion of three genes: TOP2A, SMARCA4 and TRAF4.

Further the literature is searched using pubmed in order to find genes whose overexpression in colorectal cancer had previously been shown by independent methods. Depending on the expression levels and assays used for detection, genes are scored as ‘+++’; Substantial evidence to support their overexpression, ‘++’; Significant evidence to support their overexpression, and ‘+’; Evidence to support their overexpression.

Due to improved computing power, an aim of the invention is to analyze all regulatory sequences of all differentially regulated genes. Therefore, this selection step is only optionally.

Genes, where no further evidence regarding their overexpression in colorectal cancer is found, are excluded. Finally, the regulatory regions of the following seven genes with a view to select cis-regulatory elements to form a synthetic promoter active specifically in colon cancer cells are examined: PLK, G3BP, E2-EPF, MMP9, MCM3, PRDX4 and CDC2.

2. Identification of Regulatory Elements from Upregulated Genes

Upon deciding on the genes upregulated in colorectal cancer, the nucleotide sequence of each gene (a total of seven genes) is obtained with 5 kb upstream/downstream from UCSC Golden-Path (e.g., found on the world wide web at genome.ucsc.edu) with the use of the UCSC Genome Browser on Human March 2006 Assembly.

Using the BIOBASE Biological Databases (e.g., found on the world wide web at gene-regulation.com), each retrieved sequence is BLASTed against the TRANSFAC Factor Table by using the BLASTX search tool (version 2.0.13) of the TFBLAST program (e.g., found on the world wide web at gene-regulation.com/cgi-bin/pub/programs/tfblast/tfblast.cgi) for searches against nucleotide sequences in order to identify regulatory elements. The selection of regulatory elements is based on sequence homology with significantly high (0.7-1.0) corresponding consensus sequences (identity threshold), while no restriction on score or length threshold is imposed.

The BLAST results for the genes of interest are cross-referenced in order to obtain common regulatory element lists with significant e-values (<1e-03) as well as belonging to the species of choice (Homo Sapiens). Upon further review, the colon cancer gene list showed good evidence of regulatory elements since (a) significant e-values are present in all seven genes (b) multiple common regulatory elements are present in all seven genes, (c) the majority of genes present in the colon cancer gene list are also present in other cancer gene lists (data not shown), and (d) substantial/significant evidence to support the genes overexpression are established from expression levels and assays used for detection.

The 7 gene sequences of interest from the colon cancer gene list are further investigated with the use of the PATCH public 1.0 (Pattern Search for Transcription Factor Binding Sites) (e.g., found on the world wide web at gene-regulation.com/cgi-bin/pub/programs/patch/bin/patch.cgi), from the BIOBASE Biological Databases. The search is conducted for all sites with a minimum site length of 7 bases, maximum number of mismatches of 0, mismatch penalty of 100, and lower score boundary of 100. The results of all seven gene sequences are further analyzed by grouping them all together, excluding all transcription factor binding sites except Homo sapiens. It is then proceeded to examine the frequency that each transcription factor binding site occurred in close proximity to the seven genes that are originally identified as being upregulated in colon cancer cells. In some cases, one sequence is present multiple times in proximity to a single gene under evaluation. Thus, in order to determine the frequency of occurrence of a transcription factor binding site; the sum of each time a binding site was detected in all genes is calculated and then used the sum of all binding sites present in all genes as the common denominator.

3. Selection of Regulatory Elements for Introduction into Screening Library

A total of 328 cis-regulatory sequences are identified that are present 5854 times in the seven gene sequences that are identified as being upregulated in colorectal cancer. Then those cis-regulatory sequences are identified, which are present at the highest proportion and which displayed the highest level of conservation between genes.

To accomplish this, sequences are selected for library construction according to the following two criteria:

(A) They are present in four or more of the seven genes identified by the gene expression profile screen, i.e. present in the regulatory regions of more than fifty percent of the candidate genes. (B) The cis-regulatory sequences that are present at the highest frequency in gene regulatory regions are then subsequently analyzed using the following selection criterion (SYN value): (frequency of cis-sequence)^{(1/length of cis″ sequence in bp)}>0.5

The SYN value selection criterion has the advantage to take into account that longer sequences, which may be present at lower frequencies, may actually represent a higher degree of conservation and may therefore by important in specifically driving gene expression in colon cancer cells.

The ten cis regulatory sequences with the highest SYN value are then synthesized and used to create a retroviral vector library for selection of synthetic promoters in a colorectal cancer cell line.

### 4. Construction of the Retroviral Screening Library and

Screening in Colon Cancer Cells

In order to select the promoters with the optimal activity in colorectal cancer cells a similar protocol is used to that described by Edelman et al (2000) [PNAS 97 (7), 3038-43], which is incorporated herein by reference. In brief, sense and antisense oligonucleotides corresponding to the ten selected cis elements are designed to contain a TCGA 5′ overhang after annealing. Annealed oligonucleotides are then randomly ligated together using T4 ligase and ligated oligonucleotides in the range of 0.3-1.0 kb are selected for by extraction from a 1.0% agarose gel. It is also possible to use Gateway cloning techniques. These randomly ligated oligonucleotides are then subsequently ligated to (1) a retroviral library pSmoothy vector, which is engineered to comprise wild-type left and right ITR sequence, and (2) a retroviral library pSmoothy vector, which is engineered to comprise a mutant left ITR and wild-type right ITR sequence. Both libraries had been treated with Xho I restriction enzyme and library complexity is measured by transforming 1/50th of the ligation reaction in supercompetent ToplO bacteria using an electroporator. Plasmid DNA from pSmoothy libraries with a complexity greater than 104 colonies is then expanded and used to create retroviral vectors. pSmoothy is constructed in order to select potential synthetic promoter sequences by their ability to express both GFP and neomycin in target cells. It is constructed as a self-inactivating (SIN) retroviral vector so that upon integration into the genome of transduced cells its 3′-UTR can no longer act as a promoter. The vector comprises the mucin minimal promoter which is located within the proviral genome and immediately downstream of the polylinker, where randomly ligated oligonucleotides are inserted. GFP and neomycin coding sequences are located immediately downstream of the minimal promoter and it is expression of these two genes which is used to select the potential synthetic promoter sequences with optimal activity.

Retroviral vectors are constructed by transfecting the pSmoothy library with a retroviral VSV-G envelop construct into 293 cells stably expressing Gag and Pol and allowing viral vector to be produced over a period of 48 hours. This retroviral vector library is then used to transduce HT29, DLD-1, HCT-116 and RKO colorectal cancer cells at various titers and the transduced cells are subjected to selection with 1 mg/ml G418 for a period of several weeks. The colorectal cancer cells expressing the highest amounts of GFP are then sorted using a FACS Aria cell sorter (BD) by selecting the 10% cells expressing the highest amount of GFP. This sorted population is then subject to further selection with 1 mg/ml G418 and then sorted a second time, again selecting the 10% cells expressing the highest amount of GFP ((a) HT29; (b) HT29-SYN pre-sort; (c) HT29-SYN post-sort). Genomic DNA is then prepared from sorted colorectal cancer cells and promoter sequences are rescued by PCR using the following primers that specifically hybridize to the pSmoothy vector:

SEQ ID NO: 16—SYNIS 5′-TAT CTG CAG TAG GCG CCG GAA TTC-3′

SEQ ID NO: 17—SYN1AS 5′-GCA ATC CAT GGT GGT GGT GAA ATG-3′

A typical PCR from the genomic DNA of retrovirally-transduced HT29 cells using primers SEQ ID NO: 16 and SEQ ID NO: 17 presented above, where amplification of several species occurs after the first sort (SI) with the FACS Aria. After the second sort (S2) a single product at 290 bp is amplified.

This process is then repeated using genomic DNA isolated from pSmoothy-transduced DLD-1, HCT-116 and RKO cell lines and isolated a total of 250 sequences with the potential to drive gene expression specifically in colorectal cancer cells.

Then the ability of the 140 potential colon cancer-specific synthetic enhancer elements (CRCSE) to drive expression of the LacZ reporter gene is evaluated in all colorectal cancer cell lines under investigation: HT29, DLD1, RKO and HCT116 cells. To identify how the conformation of vector effects the function of potential colon cancer-specific synthetic enhancer elements, the LacZ expression in the library having wild-type left and right ITRs is compared to the library having a mutant left ITR and wild-type right ITR. 14 synthetic promoter elements are identified that as having the capacity to drive a higher degree of LacZ expression across the four different colorectal cancer cell lines in libraries with both wild-type ITRs as compared to a mutant ITR, and are chosen for further analysis. The level of LacZ gene expression that is achieved in colorectal cancer cells (average of HT29, DLD-1, HCT-116 and RKO cells) versus HELA control cells from each of the 140 potential synthetic promoters (normalized to the level of expression obtained with the pCMV-beta control plasmid) can be determined. From these cell lines 5 lines showing activity by two independent means of testing, i.e. beta-galactosidase and staining of cells are selected.

Overall the results illustrated that the synthetic promoters constructed in this study only drive efficient gene expression in cell lines derived from patients with colorectal cancer, and in a vector with wild-type conformation. Specifically, high levels of beta-galactosidase expression is detected in HT29, RKO, HCT116, Dld-1 and Caco-2 cells, and minimal levels of gene expression is detected in Hela.

Neuro2A, MCF-7, Panc-1, CV-1 and 3T3 cells. The results are further compared with cells transfected with vectors pCMV-beta (CMV promoter) and pDRIVE-Mucl (Mucin-1 promoter; Invitrogen).

These results clearly demonstrate that the selection procedure outlined in this example is capable of generating synthetic promoters with specific activity in colon cancer cells. Expression levels of Lac Z mediated by CRCSE-1 in HT29 and Neuro2A cells transfected using Lipofectamine 2000 and stained for LacZ expression 48 hours post-transfection is assessed. Notably, control cell lines, including NEUR02A, NIH3T3, CV1, HELA and COS-7 cells, did not exhibit any expression of Lac Z when transfected with CRCSE-1. Within these sequences the following TFBS could be identified using 86% homology as criteria. In total all the sequences used show a homology of approx. 72%. The mutation is most likely introduced during the Neomycin selection procedure. Since the minimum promoter is an essential binding site there are less mutations within this region of each sequence.

It then is assessed whether the number of cis-elements present in each promoter is an important indicator of promoter strength and specificity. A process is carried out to select promoter sequences with a higher degree of stringency; i.e. to select promoters containing cis-elements with 100% homology to the input oligonucleotides. A further 82 sequences thus are subcloned from the promoter library isolated from CRC cell genomic DNA (described above) into pBluescript II KSM; the sequences of each clone are analyzed prior to expression analysis. From these 82 sequences 55 are identified containing cis-regulatory elements with 100% homology to input oligonucleotides. All these sequences comprise a Mucin-1 minimum promoter. As controls, sequences are sub-cloned from the random ligation products of all ten cis-regulatory elements prior to selection in CRC cell lines. The results showed that on average, only 2.2 cis-regulatory elements per sequence are found in unselected sequences, compared to 4.0 elements per promoter subjected to selection through the CRC cell lines (p<0.001; Mann-Whitney non-parametric test). Indeed, only 3/22 sequences in the control group contained four or more cis-regulatory elements, compared to over 31/55 promoters containing four or more cis-elements from the group subjected to selection. More-over, cis-elements with a SYN value greater than 0.6 represented 70.0% of all the elements in the 55 identified promoters, thus confirming the importance of the SYN selection formula. To correlate the presence of specific c s-regulatory elements to level and specificity of expression, 28/31 promoters are inserted into the pSmoothy retroviral vector and their ability to drive GFP expression in CRC cells compared to the HELA control cell line is monitored.

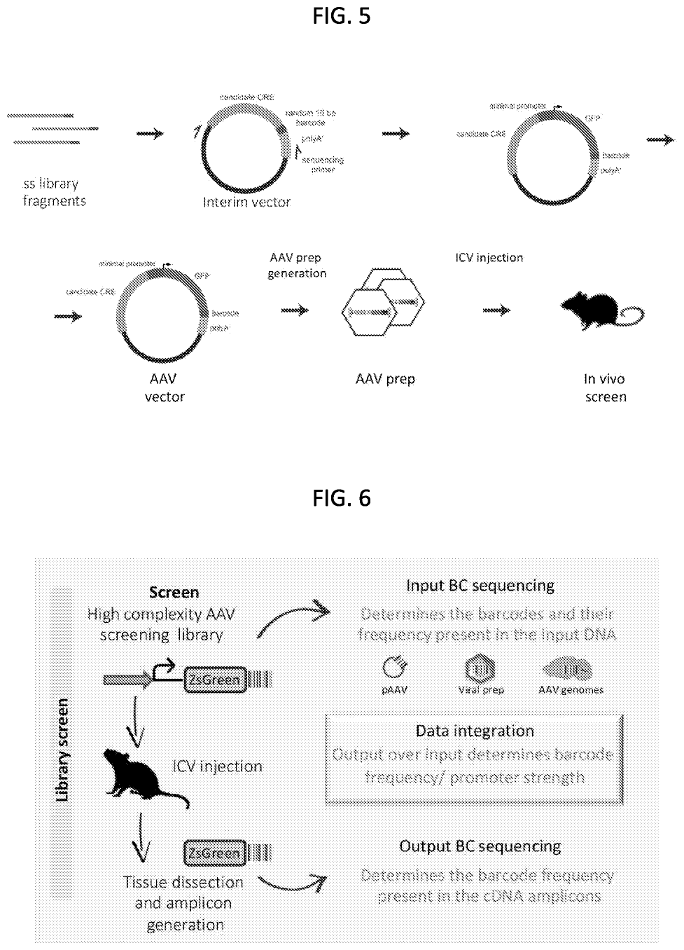

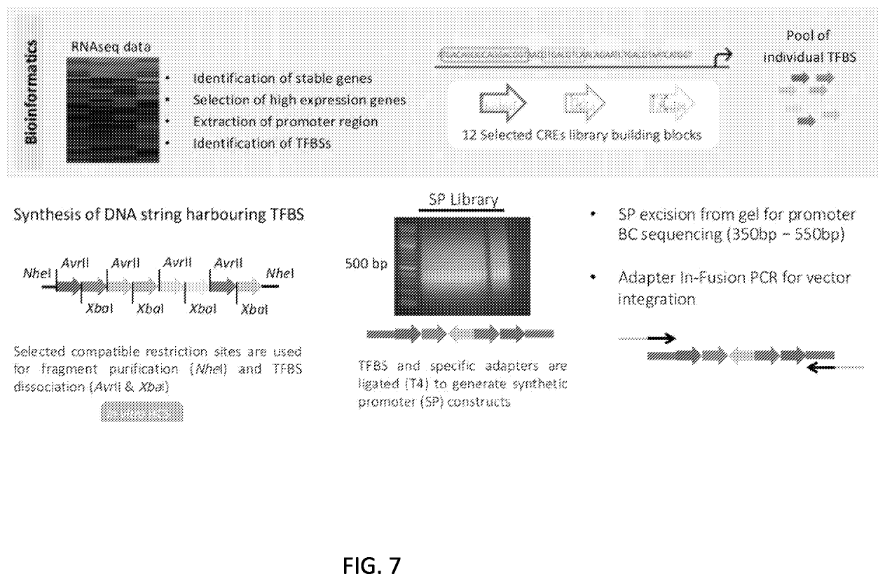

Efficiency of GFP expression is determined by FACS analysis and the proportion of cells fluorescing above a threshold value of 200 units on the FL1 channel is determined for all promoters. Depending on the cell line, an average 1.0-10.0% of the cells expressing GFP demonstrated fluorescence above this level. All promoters analyzed generated significantly higher levels of expression in CRC cell lines (HCT116, HT29, DLD1 and RKO) when compared to the HELA control cell line via, e.g., FACS; where only a small proportion of cells are GFP positive. To identify which promoters are the most efficient, an expression ratio for each promoter in all cell lines is determined; this expression ratio is defined as the proportion of cells expressing GFP above the threshold value for each individual promoter divided by the average proportion above the threshold for all promoters. The results of this analysis are shown in FIG. 6B, which illustrates that promoters 239, 213, 215, 248 and 254 show the highest activity in all CRC cell lines compared to the other promoters.

It is further examined which cis-elements constituted these more efficient promoters and found that on average the five cis-elements with the highest SYN value represented 64% of all the regulatory elements in each promoter. Thus further demonstrating the importance of the SYN value for selecting the optimal elements to maximise efficient and selective expression. Taken together the results demonstrate that the SYN selection formula and the methods provided herein represent a useful tool in selecting cis-regulatory elements (i.e., TFREs) for inclusion in synthetic promoter libraries. Several promoters are constructed using the described methodology that could efficiently express GFP or Lac Z specifically in CRC cell lines, whilst showing no or limited activity in control cells. It is specifically contemplated herein that this method can be applied in the construction of any eukaryotic promoter designed to be active in specific environmental or diseased conditions.

While the present inventions have been described and illustrated in conjunction with a number of specific embodiments, those skilled in the art will appreciate that variations and modifications may be made without departing from the principles of the inventions as herein illustrated, as described and claimed. The present inventions may be embodied in other specific forms without departing from their spirit or essential characteristics. The described embodiments are considered in all respects to be illustrative and not restrictive. The scope of the inventions is, therefore, indicated by the appended claims, rather than by the foregoing description. All changes which come within the meaning and range of equivalence of the claims are to be embraced within their scope.

1. A method of identifying the strength of one or more unique regulatory elements (URE) having conformational effect on a transcribable reporter sequence comprising:
a. expressing a plurality of synthetic nucleic acids in a population of cells, the plurality of synthetic nucleic acids comprises:
1. a first plurality of synthetic nucleic acids each comprising a unique regulatory element (URE), where the URE comprises:
i. a nucleic acid sequence containing at least one discrete regulatory element (DRE), wherein the DRE is a control (or wild type) continuous nucleic acid sequence or a control discontinuous nucleic acid sequence associated with a plurality of unique barcodes corresponding with the at least one DRE, wherein each barcode is between 12-35 nucleotides in length and has a GC content between 25-65%; and
ii. the DRE is conformationally positioned in a preselected manner relative to a nucleic acid encoding a transcribable reporter sequence, wherein if the URE does not contain a promoter, a separate promoter is operatively linked to the transcribable reporter sequence; and
2. a second plurality of synthetic nucleic acids comprising a URE that further comprises a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%;
b. determining the expression frequency of each of the plurality of corresponding barcodes in (a)(1) and (a)(2); and
c. changing in a predetermined manner the conformation of at least one of the corresponding plurality of synthetic nucleic acids' DRE relative to the transcribable reporter sequence;
d. determining the expression frequency of the at least one corresponding plurality of (c); and
e. comparing the expression frequency of (a)(1) and (a)(2) to determine the effect of the conformation change on the transcribable reporter sequence expression.

2. The method of claim 1, wherein the plurality of synthetic nucleic acids is expressed in a population of cells using a population of viral vectors.

3. The method of claim 1, wherein the DRE is proximal to or within a Holliday junction and a change in at least one of the Holliday junctions is made.

4. The method of claim 3, wherein the change in conformation is made by the addition, deletion, or substitution of one or more nucleic acids.

5. The method of claim 1, wherein at least one DRE is present in a terminal repeat (TR).

6. The method of claim 2, wherein the viral vector is a parvovirus, a lentivirus, or an adenovirus.

7. The method of claim 6, wherein the parvovirus is a dependovirus and the change in conformation is in at least one of the A, A′, B, B′, C, or C′ loops.

8. The method of claim 6, wherein the parvovirus is an adeno-associated virus (AAV) and the change in conformational is in at least one of the A, A′, B, B′, C, C′, D, D′ regions.

9. The method of claims 2 and 6, wherein the viral vector is a lentiviral vector, the DRE is TAT, and the conformational change is made in the TAR RNA stem.

10. The method of claims 2 and 6, wherein the viral vector is a lentiviral vector, the DRE is TAT, and the conformational change is made in the UU-rich bulge.

11. The method of claims 2 and 6, wherein the viral vector is a lentiviral vector, the DRE is REV, a REV Responsive Element (RRE) is present in the nucleic acid, and the conformational change is made in the RRE.

12. The method of claim 1, wherein the DRE is proximal to or within the conformation change.

13. The method of claim 1, wherein the conformational change occurs by the addition, substitution, or deletion of at least one nucleic acid.

14. The method of claim 13, wherein the addition, substitution, or deletion results in a Holliday junction.

15. The method of claim 2, wherein the plurality of synthetic nucleic acids is expressed in a population of cells in vitro using a population of AAV vectors.

16. The method of claim 2, wherein the plurality of synthetic nucleic acids is expressed in a population of cells in vivo using a population of AAV vectors.

17. A method of identifying the strength of one or more unique regulatory elements (URE) having conformational effect on a transcribable reporter sequence comprising:
a. providing a plurality of synthetic nucleic acids, wherein the plurality of synthetic nucleic acid comprises:
1. a first plurality of synthetic nucleic acids each comprising a unique regulatory element (URE), wherein the URE comprises:
i. a nucleic acid sequence containing at least one discrete regulatory element (DRE), wherein the DRE is a control (or wild type) continuous nucleic acid sequence or a discontinuous nucleic acid sequence;
ii. associated with a plurality of unique barcodes corresponding with the at least one DRE, wherein each barcode is between 12-35 nucleotides in length and has a GC content between 25-65%; and
the DRE is conformationally positioned in a preselected manner relative to a nucleic acid encoding a transcribable reporter sequence operatively linked to a promoter; wherein if the URE does not contain a promoter, a separate promoter is operatively linked to the transcribable reporter sequence; and
2. a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%;
b. generating a library of plasmids or expression vectors by inserting the plurality of synthetic nucleic acids into a plurality of plasmids or expression vectors, wherein each resulting plasmid or expression vector comprises a single synthetic nucleic acid;
c. introducing the library of plasmids or expression vectors of step (b) into a population of cells;
d. determining the expression frequency of each of the plurality of corresponding barcodes in (a) (1) and (a) (2); and
e. comparing the expression frequency of (a)(1) and (a)(2) to determine the effect of the conformation change on the transcribable reporter sequence expression.

18. A method of identifying the strength of one or more unique regulatory elements (URE) having conformational effect on a transcribable reporter sequence comprising:
a. providing the plurality of synthetic nucleic acids, wherein the plurality of synthetic nucleic acid comprises:
1. a unique regulatory element (URE), wherein the URE comprises:
i. a first plurality of synthetic nucleic acid sequences each containing at least one discrete regulatory element (DRE), wherein the DRE is a control (or wild type) continuous nucleic acid sequence or a discontinuous nucleic acid sequence;
ii. associated with a plurality of unique barcodes corresponding with the at least one DRE, wherein each barcode is between 12-35 nucleotides in length and has a GC content between 25-65%; and
the DRE is positioned in a preselected manner relative to a nucleic acid encoding a transcribable reporter sequence operatively linked to a promoter; wherein if the URE does not contain a promoter, a separate promoter is operatively linked to the transcribable reporter sequence; and
2. a second plurality of synthetic nucleic acids comprising a URE further comprising a change in the conformation of said at least one DRE of a(1)(ii) relative to the transcribable reporter sequence wherein the conformationally changed DRE is associated with a plurality of unique barcodes different than in (1)(i), wherein each barcode is between 12-35 nucleotides in length and has a GC content between 25-65%;
b. generating a library of plasmids or expression vectors by inserting the plurality of synthetic nucleic acids into a plurality of plasmids or expression vectors, wherein each resulting plasmid or expression vector comprises a single synthetic nucleic acid;
c. introducing the library of plasmids or expression vectors of step (b) into an AAV vector to form an AAV vector library;
d. introducing the AAV vector library into a population of cells;
e. determining the expression frequency of each of the corresponding barcodes of (a)(1) and (a)(2)
f. comparing the expression frequency of (a)(1) and (a)(2) to determine the effect of the conformation change on the strength of expression.

19. The method of claim 1, further comprising the step of, after step (a), waiting a sufficient amount of time for expression of the plurality of synthetic nucleic acids in the population of cells.

20. The method of any of claims 17-18, further comprising the step of, after step (c), waiting a sufficient amount of time for expression of the library of plasmids or expression vectors of step (b).

21. The method of any of claims 1, 17, or 18, wherein determining includes the steps of:
a. obtaining mRNA from the population of cells;
b. synthesizing cDNA from the mRNA of step (a);
c. amplifying a region of nucleic acids (amplicon) from the cDNA of step (b); and
d. measuring the expression frequency of each of the plurality of barcodes in the amplicon of step (c).

22. The method of claim 21, wherein measuring is performed by sequencing.

23. The method of any of claims 1, 17, or 18, wherein the expression frequency of each of the plurality of barcodes is the normalized to a barcode input, and wherein the barcode input is each unique barcode content before expression.

24. The method of claim 21, wherein the expression frequency of the barcode measured in the amplicon is a barcode output.

25. The method of any of the preceding claims, wherein at least one DRE is a discontinuous DRE.

26. The method of claim 25, wherein the discontinuous DRE comprises a portion of the DRE located 5′ of the transcribable reporter sequence, and a portion of the DRE located 3′ of the transcribable reporter sequence.

27. The method of claim 25 or 26, wherein the discontinuous DRE comprises a non-DRE nucleic acid sequence located in a 5′- or 3′-portion of the DRE.

28. The method of any of the preceding claims, wherein the at least one DRE is located within 200-500 bp of the at least one TR, or portion thereof.

29. The method of any of the preceding claims, wherein the at least one DRE is located within 20-200 bp of the at least one TR, or portion thereof.

30. The method of any of the preceding claims, wherein the at least one DRE is located within 20 bp of the at least one TR, or portion thereof.

31. The method of any of the preceding claims, wherein the URE strength is measured in the same system from which it is derived.

32. The method of claim 25, wherein at least part of the at least one discontinuous DRE includes a TR.

33. The method of any of the previous claims, wherein the at least one TR, or portion thereof, comprises at least one modification.

34. The method of any of the previous claims, wherein the at least one TR comprises at least 1, 2, 3, 4, 5, 6, or more modifications.

35. The method of any of the previous claims, wherein the at least 1, 2, 3, 4, 5, 6, or more modifications are associated with the same plurality of unique barcodes as in claims 1(a)(2), 17(a)(2) or 18(a)(2).

36. The method of any of the previous claims, wherein the synthetic nucleic acid contains at least 2, 3, 4, 5, 6, or more TRs, or portion thereof.

37. The method of any of claim 25, wherein the synthetic nucleic acid contains at least 2, 3, 4, 5, 6, or more discontinuous DREs.

38. The method of any of claims 1, 17, or 18, wherein the URE comprises at least DRE selected from the group consisting of: a promoter, a transcription factor binding site, an enhancer, a silencer, a boundary control element, an insulator, a locus control region, a response element, a binding site, a segment of a terminal repeat, a responsive site, a stabilizing element, a de-stabilizing element, and a splicing element.

39. The method of any of claims 1, 17, or 18, wherein the nucleic acid sequence containing at least one DRE comprises a combination of DREs.

40. The method of any of claim 39, wherein the combination of DREs contain at least 2, 3, 4, 5, 6, or more regulatory sequence elements.

41. The method of any of claim 40, wherein the combination of DREs is associated with the same plurality of unique barcodes of any of claims 1, 17, or 18.

42. The method of claim 2, wherein the viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector

43. The method of any of claim 18 or 42, wherein the AAV vector is a AAV serotype selected from the group consisting of: 1, 2, 3a, 3b, 4, 5, 6, 7, 8, 9, 10, 11, and 13.

44. The method of any of claim 1 or 18, wherein the synthetic nucleic acid comprises an inverted terminal repeat (ITR), or a portion thereof.

45. The method of any of claim 2, wherein the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a TRS (terminal resolution site), and a Rep binding site (RBS).

46. The method of claim 45, wherein the ITR is a wild-type inverted terminal repeat (ITR), a mutant ITR, or a synthetic ITR, wherein the mutant or synthetic ITR comprises a modification as compared to the wild-type ITR sequence.

47. The method of claim 45, wherein the A region, A′ region, B region, B′ region, C region, C′ region, D region, or D′ region is derived from a wild-type inverted terminal repeat (ITR), a mutant ITR, a truncated ITR, or a synthetic ITR.

48. The method of any of claim 5, wherein the TR is a long terminal repeat (LTR), or a portion thereof.

49. The method of claim 46, wherein the modification is a base pair insertion, deletion, mutation, truncation, or substitution as compared to the wild-type ITR sequence.

50. The method of any of the previous claims, wherein the at least one DRE and the TR sequence are separated by 1-500 base pairs.

51. The method of any of the previous claims, wherein each portion of a discontinuous DRE (dcDRE) is separated by 1-500 base pairs.

52. The method of any of the previous claims, wherein each portion of a discontinuous DRE (dcDRE) is separated by at least 50 base pairs.

53. The method of any of the previous claims, wherein one portion of a discontinuous DRE (dcDRE) can be 5′ of the transcribable reporter sequence, and a second portion of the dcDRE is 3′ of the transcribable reporter sequence.

54. The method of any of the previous claims, wherein the transcribable reporter sequence is the open reading frame (ORF) of a marker gene.

55. The method of claim 54, wherein the marker gene encodes a fluorescent protein, a luminescent protein, or an element tag.

56. The method of any of claims 1, 17 or 18, wherein the barcode contains at least one of each: adenine, thymine, guanine, and cytosine.

57. The method of any of claims 1, 17 or 18, wherein the barcode is a semi-degenerate barcode.

58. The method of any of claims 1, 17 or 18, wherein the barcode does not contain tracts of more than three homopolymers in succession.

59. The method of any of claims 1, 17 or 18, wherein the barcode does not contain the nucleic acid sequence of a restriction enzyme.

60. The method of any of claims 1, 17 or 18, wherein the barcode has a hamming distance greater than 2.

61. The method of any of claims 1, 17 or 18, wherein the barcode is between 12-25 nucleotides in length.

62. The method of any of claims 1, 17 or 18, wherein the barcode is between 12-28 nucleotides in length.

63. The method of any of claims 1, 17 or 18, wherein the barcode has a complexity of at least 4.3×10⁷, at least 2.7×10⁸, or at least 1×10¹².

64. The method of any of claims 1, 17 or 18, wherein a plurality of barcodes comprises at least 2, 3, 4, 5, 6, 7, 8, 9, 10, or more barcodes.

65. The method of any of claims 1, 17 or 18, wherein a plurality of barcodes comprises 2-20 barcodes.

66. The method of any of claims 1, 17 or 18, wherein the synthetic nucleic acid is further modified for next generation sequencing.

67. The method of any of claims 1, 17 or 18, wherein the synthetic nucleic acid comprises at least one unique molecular identifier (UMI) and at least one unique primer annealing sites (UPAS) tag.

68. A plurality of at least 50 synthetic nucleic acids, each synthetic nucleic acid comprising a URE, where the URE comprises:
a. a nucleic acid sequence containing at least one discrete regulatory element (DRE), wherein the DRE is a continuous nucleic acid sequence or a discontinuous nucleic acid sequence;
b. a nucleic acid sequence encoding an open reading frame;
c. a nucleic acid sequence encoding a viral vector terminal repeat (TR); and
d. a plurality of unique barcodes associated with the at least one DRE,
wherein each barcode has a GC content between 25-65%.

69. A plurality of at least 50 synthetic nucleic acids, each synthetic nucleic acid comprising a URE, where the URE comprises:
a. a nucleic acid sequence containing at least one discrete regulatory element (DRE), wherein the DRE is a continuous nucleic acid sequence or a discontinuous nucleic acid sequence;
b. a nucleic acid sequence encoding an open reading frame;
c. a nucleic acid sequence encoding at least one partial viral vector comprising at least a part of a terminal repeat (TR); and
d. a plurality of unique barcodes associated with the at least one DRE,
wherein each barcode is between 12-35 nucleotides in length and have a GC content between 25-65%.

70. The plurality of synthetic nucleic acids of any of claim 68 or 69, wherein the DRE comprises at least one regulatory sequence element selected from the group consisting of: a promoter, a transcription factor binding site, an enhancer, a silencer, a boundary control element, an insulator, a locus control region, a response element, a binding site, a segment of a terminal repeat, a responsive site, a stabilizing element, a de-stabilizing element, and a splicing element.

71. The plurality of synthetic nucleic acids of claim 68 or 69, wherein the nucleic acid sequence containing at least one DRE comprises a combination of DREs.

72. The plurality of synthetic nucleic acids of claim 71, wherein the combination of DREs contain 2-6 DREs.

73. The plurality of synthetic nucleic acids of claim 71, wherein the combination of regulatory sequence elements is associated with the same plurality of unique barcodes of claims 68 and 69.

74. The plurality of synthetic nucleic acids of claim 68 or 69, wherein at least part of the at least one DRE includes a TR.

75. The plurality of synthetic nucleic acids of claim 68 or 69, wherein the synthetic nucleic acid contains at least 2 TRs.

76. The plurality of synthetic nucleic acids of claim 68 or 69, wherein the at least one discontinuous regulatory element comprises at least one modification.

77. The plurality of synthetic nucleic acids of claim 68 or 69, wherein the viral vector comprises at least 4 modifications.

78. The plurality of synthetic nucleic acids of claim 56 or 57, wherein the viral vector is selected from the group consisting of: an AAV vector, an adenovirus vector, a lentivirus vector, a retrovirus vector, a herpesvirus vector, an alphavirus vector, a poxvirus vector, a baculovirus vector, and a chimeric virus vector

79. The plurality of synthetic nucleic acids of claim 78, wherein the AAV vector is a AAV serotype selected from the group consisting of: 1, 2, 3a, 3b, 4, 5, 6, 7, 8, 9, 10, 11, and 13.

80. The plurality of synthetic nucleic acids of claims 68, 69, or 74, wherein the TR is an inverted terminal repeat (ITR).

81. The plurality of synthetic nucleic acids of claim 80, wherein the viral vector is an AAV vector and the at least a part of a terminal repeat (TR) is selected from the group consisting of: an inverted terminal repeat (ITR), an A region, an A′ region, a B region, a B′ region, a C region, a C′ region, a D region, a D′ region, a spacer sequence, a CAP gene sequence, a Rep gene sequence, a Rep Binding Site, and a terminal resolution site.

82. The plurality of synthetic nucleic acids of claim 80 or 81, wherein the ITR is a wild-type inverted terminal repeat (ITR), a mutant ITR, or a synthetic ITR

83. The plurality of synthetic nucleic acids of claim 81, wherein the A region, A′ region, B region, B′ region, C region, C′ region, D region, or D′ region is derived from a wild-type inverted terminal repeat (ITR), a mutant ITR, a truncated ITR, or a synthetic ITR.

84. The plurality of synthetic nucleic acids of claims 68, 69, or 74, wherein the TR is a long terminal repeat (LTR).

85. The plurality of synthetic nucleic acids of any of claim 76 or 77, wherein the modification is a base pair insertion, deletion, mutation, truncation, or substitution as compared to the wild-type sequence.

86. The plurality of synthetic nucleic acids of claim 68 or 69, wherein the DRE and the TR comprised in the viral vector or the partial vector are separated by 2-500 base pairs.

87. The plurality of synthetic nucleic acids of claim 72, wherein the DREs are separated by 2-200 base pairs.

88. The plurality of synthetic nucleic acids of claim 68 or 69, wherein the open reading frame is the open reading frame of a marker gene.

89. The plurality of synthetic nucleic acids of claim 89, wherein the marker gene encodes a fluorescent protein, a luminescent protein, or an element tag.

90. The plurality of synthetic nucleic acids of claim 68 or 69, wherein the barcode contains at least one of each: adenine, thymine, guanine, and cytosine.

91. The plurality of synthetic nucleic acids of claim 68 or 69, wherein the barcode is a semi-degenerate barcode.

92. The plurality of synthetic nucleic acids of claim 68 or 69, wherein the barcode does not contain tracts of more than three homopolymers in succession.

93. The plurality of synthetic nucleic acids of claim 68 or 69, wherein the barcode does not contain the nucleic acid sequence of a restriction enzyme.

94. The plurality of synthetic nucleic acids of claim 68 or 69, wherein the barcode has a hamming distance greater than 2.

95. The plurality of synthetic nucleic acids of claim 68 or 69, wherein the barcode is between 12-28 nucleotides in length.

96. The plurality of synthetic nucleic acids of claim 68 or 69, wherein the barcode is between 12-25 nucleotides in length.

97. The plurality of synthetic nucleic acids of claim 68 or 69, wherein the barcode has a complexity of at least 4.3×10⁷, at least 2.7×10⁸, or at least 1×10¹².

98. The plurality of synthetic nucleic acids of claim 68 or 69, wherein a plurality of barcodes comprises at least 2 barcodes.

99. The plurality of synthetic nucleic acids of claim 68 or 69, wherein a plurality of barcodes comprises at least 2, 3, 4, 5, 6, 7, 8, 9, 10, or more barcodes.

100. The plurality of synthetic nucleic acids of claim 68 or 69, wherein the synthetic nucleic acid is further modified for next generation sequencing.

101. The plurality of synthetic nucleic acids of claim 68 or 69, wherein the synthetic nucleic acid comprises at least one UMI and at least one UPAS.

102. A library of at least 50 plasmids expressing the plurality of synthetic nucleic acids of any of claims 1-4.

103. A library of at least 50 expression vectors comprising the plurality of synthetic nucleic acids of any of claims 1-4.

104. The library of claim 102 or 103, wherein the library comprises control plasmids or control expression vectors.

105. A population of cells comprising the library of any of claim 102 or 103.

106. The population of cells of claim 105, wherein the cells are eukaryotic, prokaryotic, viral, or bacterial.

107. The population of cells of claim 105, wherein the synthetic nucleic acids, plasmids, or expression vectors is transiently expressed.

108. The population of cells of claim 105, wherein the synthetic nucleic acids, plasmids, or expression vectors is stably expressed.

109. A population of at least 50 viral vectors expressing the plurality of synthetic nucleic acids of claims 1-4, the library of plasmids of claim 102, or the library of expression vectors of claim 103.

110. The population of viral vectors of claim 109, wherein the viral vector is an AAV vector.

111. A method of identifying the strength of a URE from a plurality of UREs in vitro, the method comprising:
a. expressing the plurality of synthetic nucleic acids of any of claim 68 or 69, the library of plasmids of claim 102, or the library of expression vectors of claim 103 in a population of cells; and
b. determining the expression frequency of each of the plurality of barcodes,
wherein the expression frequency of each of the plurality of barcodes is an indicator of the strength of the associated URE.

112. A method of identifying the strength of a URE from a plurality of UREs in vitro, the method comprising:
a. providing the plurality of synthetic nucleic acids of claim 68 or 69;
b. inserting the plurality of synthetic nucleic acids into a library of plasmids or expression vectors, wherein the resulting plasmid or expression vector each comprise at least one DRE, an open reading frame, a viral vector terminal repeat (TR) or at least one partial viral vector comprising at least a part of a terminal repeat (TR), and a plurality of barcodes associated with at least one DRE;
c. introducing the library of plasmids or expression vectors of step (b) into a population of cells; and
d. determining the expression frequency of the plurality of barcodes,
wherein the expression frequency of each of the plurality of barcodes is an indicator of strength of the URE.

113. A method of identifying the strength of a URE from a plurality of UREs in vitro, the method comprising:
a. providing the plurality of synthetic nucleic acids of claim 68 or 69;
b. inserting the plurality of synthetic nucleic acids into a library of plasmids or expression vectors, wherein the resulting plasmid or expression vector each comprise at least one DRE, an open reading frame, a viral vector terminal repeat (TR) or at least one partial viral vector comprising at least a part of a terminal repeat (TR), and a plurality of barcodes associated with the at least one DRE;
c. introducing the plurality of plasmids or expression vectors of step (b) into an AAV vector to form AAV vector library;
d. introducing the AAV vector library into a population of cells; and
e. determining the expression frequency of the plurality of barcodes,
wherein the expression frequency of each of the plurality of barcodes is an indicator of the strength of the URE.

114. The method of any of claims 112-113, further comprising the step of, after step (c) of claim 112 or after step (d) of claim 113 waiting a sufficient amount of time for expression of the synthetic nucleic acids, the plasmids, or the expression vectors.

115. The method of any of claims 111-114, wherein determining the expression frequency includes the steps of:
a. obtaining mRNA from the population of cells;
b. synthesizing cDNA from the mRNA of step (a);
c. amplifying a region of nucleic acids (amplicon) from the cDNA of step (b); and
d. measuring the expression frequency of each of the plurality of barcodes in the amplicon of step (c).

116. The method of claim 115, wherein measuring is performed by sequencing.

117. The method of any of claims 111-116, wherein is the expression frequency of the barcode measured in the amplicon is a barcode output.

118. The method of any of claim 117, wherein the barcode output is the normalized to a barcode input, and wherein the barcode input is each unique barcode content before expression.

119. A method of identifying the strength of a URE from a plurality of UREs in vivo, the method comprising:
a. administering the population of viral vectors of claims 109-110 in vivo; and
b. determining the expression frequency of each of the plurality of barcodes,
wherein the expression frequency of each of the plurality of barcodes is an indicator of the strength of the associated URE.

120. A method of identifying the strength of a URE from a plurality of UREs, the method comprising:
a. providing the plurality of synthetic nucleic acids of any of claim 68 or 69;
b. inserting the plurality of synthetic nucleic acids into a library of plasmids or expression vectors, wherein the resulting plasmid or expression vector each comprise a single synthetic nucleic acid;
c. introducing the plurality of plasmids or expression vectors of step (b) into an viral vector;
d. administering the resulting viral vector of step (c) in vivo; and
e. determining the expression frequency of each of the plurality of barcodes,
wherein the expression frequency of each of the plurality of barcodes is an indicator of the strength of the associated URE.

121. The method of claims 119-120, wherein the viral vector is an AAV vector.

122. The method of claims 119-120, further comprising the step of, after administering, waiting a sufficient amount of time for expression of the synthetic nucleic acids, the plasmids, or the expression vectors.

123. The method of claim 119-120, wherein determining the expression frequency includes the steps of:
a. obtaining mRNA from tissues or cells of interest after in vivo administration of viral vectors;
b. synthesizing cDNA from the mRNA of step (a);
c. amplifying a region of nucleic acids (amplicon) from the cDNA of step (b); and
d. measuring the expression frequency of each of the plurality of barcodes in the amplicon of step (c).

124. The method of claim 123, wherein measuring is performed by sequencing.

125. The method of claim 123, wherein is the expression frequency of the barcode measured in the amplicon is a barcode output.

126. The method of claim 125, wherein the barcode output is normalized to a barcode input, and wherein the barcode input is each unique barcode content before expression.

127. The method of any of the preceding claims, wherein the URE strength is measured in the same system from which it is derived.

128. A plurality of at least 50 synthetic nucleic acids, each synthetic nucleic acid comprising:
a. a nucleic acid sequence containing at least one discrete regulatory element (DRE);
b. a nucleic acid sequence encoding an open reading frame;
c. a nucleic acid sequence encoding a viral vector; and
d. a plurality of unique barcodes associated with the at least one DRE,
wherein each barcode is between 12-35 nucleotides in length and have a GC content between 25-65%.

129. A plurality of at least 50 synthetic nucleic acids, each synthetic nucleic acid comprising:
a. a nucleic acid sequence containing at least one discrete regulatory element (DRE);
b. a nucleic acid sequence encoding an open reading frame;
c. a nucleic acid sequence encoding at least one partial viral vector; and
d. a plurality of unique barcodes associated with the at least one DRE,
wherein each barcode is between 12-35 nucleotides in length and have a GC content between 25-65%.

130. The plurality of synthetic nucleic acids of claims 128-129, wherein the viral vector comprises 1-6 modifications.

131. The plurality of synthetic nucleic acids of claim 131, wherein the 1-6 modifications are associated with the same plurality of unique barcodes of claims 128-129.

132. The plurality of synthetic nucleic acids of claim 129, wherein the partial viral vector is selected from the group consisting of: a terminal repeat, response element, cis-acting viral element, and a trans-acting viral element.

133. The method of any of claims 1, 4, 7-13, 17, or 18, wherein the conformational change is not determined.

134. The method of any of claims 1, 4, 7-13, 17, or 18, wherein the conformational change determined by assessing the at least one mutation against a non-altered sequence under the same condition.


SEQUENCE LISTING (34 sequences)

SEQ ID NO: 1 - DNA, 9 bp, Unknown
  Description of Unknown TF target sequence
tgacgtgct

SEQ ID NO: 2 - DNA, 7 bp, Artificial Sequence
  Description of Artificial Sequence Synthetic oligonucleotide
tgacgtg

SEQ ID NO: 3 - DNA, 20 bp, Artificial Sequence
  Description of Artificial Sequence Synthetic oligonucleotide
gatgatgcgtagctagtagt

SEQ ID NO: 4 - DNA, 7 bp, Unknown
  Description of Unknown TF target sequence
tgacgtg

SEQ ID NO: 5 - DNA, 84 bp, Artificial Sequence
  Description of Artificial Sequence Synthetic oligonucleotide
aggtctatataagcagagctcgtttagtgaaccgtcagatcgcctagatacgccatccacgctgttttgacctccatagaagat

SEQ ID NO: 6 - DNA, 81 bp, Artificial Sequence
  Description of Artificial Sequence Synthetic oligonucleotide
gcagttagcgtagctgaggtaccgtcgacgatatcggatccttcgcatattaaggtgacgcgtgtggcctcgaacaccgag

SEQ ID NO: 7 - DNA, 64 bp, Artificial Sequence
  Description of Artificial Sequence Synthetic primer
caagcagaagacggcatacgagatacgagactgattagtcagtcagccctccgccttgccctga

SEQ ID NO: 8 - DNA, 65 bp, Artificial Sequence
  Description of Artificial Sequence Synthetic primer
aatgatacggcgaccaccgagatctacactatggtaattgttcctactattccgtaccgtagggt

SEQ ID NO: 9 - DNA, 64 bp, Artificial Sequence; identical to SEQ ID NO: 7
  Description of Artificial Sequence Synthetic primer

SEQ ID NO: 10 - DNA, 65 bp, Artificial Sequence; identical to SEQ ID NO: 8
  Description of Artificial Sequence Synthetic primer

SEQ ID NO: 11 - DNA, 21 bp, Artificial Sequence
  Description of Artificial Sequence Synthetic primer
ttcgatcaactacgcagacag

SEQ ID NO: 12 - DNA, 22 bp, Artificial Sequence
  Description of Artificial Sequence Synthetic primer
gtccgtgagtgaagcagatatt

SEQ ID NO: 13 - DNA, 24 bp, Artificial Sequence
  Description of Artificial Sequence Synthetic probe
tctgatgctgtttccctgcagaca

SEQ ID NO: 14 - DNA, 64 bp, Artificial Sequence; identical to SEQ ID NO: 7
  Description of Artificial Sequence Synthetic primer

SEQ ID NO: 15 - DNA, 65 bp, Artificial Sequence; identical to SEQ ID NO: 8
  Description of Artificial Sequence Synthetic primer

SEQ ID NO: 16 - DNA, 24 bp, Artificial Sequence
  Description of Artificial Sequence Synthetic primer
tatctgcagtaggcgccggaattc

SEQ ID NO: 17 - DNA, 24 bp, Artificial Sequence
  Description of Artificial Sequence Synthetic primer
gcaatccatggtggtggtgaaatg

SEQ ID NO: 18 - DNA, 68 bp, Artificial Sequence
  Description of Artificial Sequence Synthetic primer
caagcagaagacggcatacgagatacgagactgattagtcagtcagcccaaagaccccaacgagaagc

SEQ ID NO: 19 - DNA, 68 bp, Artificial Sequence
  Description of Artificial Sequence Synthetic primer
caagcagaagacggcatacgagatgctgtacggattagtcagtcagcccaaagaccccaacgagaagc

SEQ ID NO: 20 - DNA, 68 bp, Artificial Sequence
  Description of Artificial Sequence Synthetic primer
caagcagaagacggcatacgagatatcaccaggtgtagtcagtcagcccaaagaccccaacgagaagc

SEQ ID NO: 21 - DNA, 68 bp, Artificial Sequence
  Description of Artificial Sequence Synthetic primer
caagcagaagacggcatacgagattggtcaacgataagtcagtcagcccaaagaccccaacgagaagc

SEQ ID NO: 22 - DNA, 68 bp, Artificial Sequence
  Description of Artificial Sequence Synthetic primer
caagcagaagacggcatacgagatatcgcacagtaaagtcagtcagcccaaagaccccaacgagaagc

SEQ ID NO: 23 - DNA, 68 bp, Artificial Sequence
  Description of Artificial Sequence Synthetic primer
caagcagaagacggcatacgagatgtcgtgtagcctagtcagtcagcccaaagaccccaacgagaagc

SEQ ID NO: 24 - DNA, 68 bp, Artificial Sequence
  Description of Artificial Sequence Synthetic primer
caagcagaagacggcatacgagatagcggaggttagagtcagtcagcccaaagaccccaacgagaagc

SEQ ID NO: 25 - DNA, 68 bp, Artificial Sequence
  Description of Artificial Sequence Synthetic primer
caagcagaagacggcatacgagatatcctttggttcagtcagtcagcccaaagaccccaacgagaagc

SEQ ID NO: 26 - DNA, 68 bp, Artificial Sequence
  Description of Artificial Sequence Synthetic primer
caagcagaagacggcatacgagattacagcgcatacagtcagtcagcccaaagaccccaacgagaagc

SEQ ID NO: 27 - DNA, 68 bp, Artificial Sequence
  Description of Artificial Sequence Synthetic primer
caagcagaagacggcatacgagataccggtatgtacagtcagtcagcccaaagaccccaacgagaagc

SEQ ID NO: 28 - DNA, 61 bp, Artificial Sequence
  Description of Artificial Sequence Synthetic primer
aatgatacggcgaccaccgagatctacactatggtaattgtgccccgactctaggaattca

SEQ ID NO: 29 - DNA, 46 bp, Artificial Sequence
  Description of Artificial Sequence Synthetic oligonucleotide
caagcagaagacggcatacgagatacgagactgattagtcagtcag

SEQ ID NO: 30 - DNA, 39 bp, Artificial Sequence
  Description of Artificial Sequence Synthetic oligonucleotide
aattaccatagtgtagatctcggtggtcgccgtatcatt

SEQ ID NO: 31 - DNA, 53 bp, Artificial Sequence
  Description of Artificial Sequence Synthetic oligonucleotide
atgacagggcaggacggtaacgtgacgtcaacagaatctgacgtaatcatggt

SEQ ID NO: 32 - DNA, 20 bp, Artificial Sequence
  Description of Artificial Sequence Synthetic oligonucleotide
agcttttttttttttttttt

SEQ ID NO: 33 - DNA, 21 bp, Artificial Sequence
  Description of Artificial Sequence Synthetic oligonucleotide
acgtatagttgaggtcacgta

SEQ ID NO: 34 - DNA, 15 bp, Artificial Sequence
  Description of Artificial Sequence Synthetic oligonucleotide
ggtcagtacctagcg